(19) United States
(12) Patent Application Publication
(10) Pub. No.: US 2026/0335232 A1
(43) Pub. Date: Oct. 29, 2026

(54) TAILORED HYPOIMMUNOGENIC BIOMIMETIC NANOVESICLE DELIVERY SYSTEM FOR VIRAL INFECTION

(71) Applicant: Thomas MALCOLM, Andover, NJ (US)

(72) Inventor: Thomas MALCOLM, Andover, NJ (US)

(21) Appl. No.: 18/868,258

(22) PCT Filed: May 25, 2023
(86) PCT No.: PCT/US2023/023531

§ 371 Date: Nov. 22, 2024

Related U.S. Application Data
(60) Provisional application No. 63/345,649, filed on May 25, 2022.
(60) Provisional application No. 63/345,643, filed on May 25, 2022.
(60) Provisional application No. 63/410,881, filed on Sep. 28, 2022.
(60) Provisional application No. 63/410,874, filed on Sep. 28, 2022.

(51) Int. Cl. A61K 9/1271; A61K 31/4196; A61K 38/17; A61K 40/31; A61K 40/42; B82Y 5/00; C07K 14/435; C07K 14/705; C07K 16/28; C12N 5/078; C12N 9/64

(52) U.S. Cl. CPC A61K 9/1271; A61K 31/4196; A61K 38/17; A61K 40/31; A61K 40/421; C07K 14/435; C07K 14/70596; C07K 16/2803; C07K 16/2818; C07K 16/2827; C12N 5/0634; C12N 9/6408; A61K 2239/13; A61K 2239/21; A61K 2239/22; B82Y 5/00; C07K 2317/622; C12N 2510/00; C12Y 304/21078; C12Y 304/21079

(57) ABSTRACT

Disclosed herein are compositions comprising allogeneic, hypoimmunogenic chimeric antigen receptor (CAR), viral epitope recognition receptor (VERR), and/or viral ligand-targetable biomimetic nanovesicles (BioNVs) and methods of using the same for the treatment, prevention, and/or amelioration of viral diseases.

### CROSS-REFERENCE TO RELATED APPLICATIONS

This application claims the benefit of and priority to U.S. Provisional Application No. 63/345,643, filed May 25, 2022, U.S. Provisional Application No. 63/345,649, filed May 25, 2022, U.S. Provisional Application No. 63/410,874, filed Sep. 28, 2022, and U.S. Provisional Application No. 63/410,881, filed Sep. 28, 2022, the contents of which are herein incorporated by reference in their entirety.

### TECHNICAL FIELD

The present disclosure provides, in part, compositions and methods comprising allogeneic, hypoimmunogenic biomimetic nanovesicles and methods of using the same for the treatment or prevention of a viral disease or disorder, e.g., in a mammalian subject, such as a human.

### BACKGROUND

In people living with viral infection such as Human Immunodeficiency Virus (HIV) (PLWH) on antiretroviral therapy (ART), the virus persists in a latent form where there is minimal transcription or protein expression. Latently infected cells are a major barrier to curing HIV, among curing other viruses with latency phases. Recent studies have determined that programmed cell death protein 1 (PD-1) expressing CD4+ and CD8+ T cells are preferentially infected with HIV latency in vivo (Uldrick, T S, et al. “Pembrolizumab induces HIV latency reversal in people living with HIV and cancer on antiretroviral therapy.” Sci Transl Med. 14; 629 (2022): eabl3836). PD-1 is an inhibitory checkpoint molecule expressed on T cells that inhibits immune responses against cancers and viral infections. Monoclonal antibodies targeting PD-1 or its ligand, PD-L1, are approved to treat a growing number of cancers, including several HIV-associated malignancies (Lurain, K et al. “Anti-PD-1 and anti-PD-L1 monoclonal antibodies in people living with HIV and cancer.” Curr HIV/AIDS Rep. 17 (2020): 547-556).

Although there are infection management options for latently infectious viruses such as HIV, there is presently a paucity of effective curative treatments for these, and many other viral diseases. Several caveats exist in developing treatment options which can fully eliminate latently infected cells, for example, targeting of therapeutics to diseased cells lacking viral antigens. Cell-based therapies, such as chimeric antigen receptor engineered T cells (CAR-T cells) are currently in development for treatment of many cancers and infectious diseases, owning to the enhanced targeting of specific cell subsets. However, in developing cell therapies, there are inherent obstacles in the way of immune incompatibilities, immune exhaustion, poor cell penetrance (such as in solid tumor environments), manufacturing costs, autologous cell sources, regulation of cytokine production, etc. Therefore, there remains a need for therapies that are useful for treating diseases that take advantage of viral ligand cell targeting while overcoming the shortcomings of cell-based therapies.

### SUMMARY

In aspects, the present invention relates to a method of treating or preventing a viral infection, comprising administering to a subject in need thereof a therapeutically effective amount of (a) a biomimetic nanovesicle (BioNV), the BioNV being targeted to one or more markers of cellular infection with the virus, and (b) one or more checkpoint inhibiting agents.

In aspects, the present invention relates to a method of treating or preventing a viral infection, comprising administering to a subject in need thereof a therapeutically effective amount of a biomimetic nanovesicle (BioNV), the BioNV being targeted to one or more markers of cellular infection with the virus, wherein the subject is undergoing treatment with one or more checkpoint inhibiting agents.

In aspects, the present invention relates to a method of treating or preventing a viral infection, comprising administering to a subject in need thereof a therapeutically effective amount of one or more checkpoint inhibiting agents, wherein the subject is undergoing treatment with a biomimetic nanovesicle (BioNV), the BioNV being targeted to one or more markers of cellular infection with the virus.

In aspects, the present invention relates to methods of treating or preventing a viral infection, comprising administering to a subject in need thereof a therapeutically effective amount of a biomimetic nanovesicle (BioNV), the BioNV: (i) being targeted to one or more markers of cellular infection with the virus; and (ii) comprising one or more checkpoint inhibiting agents.

In embodiments, the marker is expressed on one or more of T cells, dendritic cells, macrophages, or any cell type in the HIV-infected cell reservoir. In embodiments, the T cells are CD4+ T cells or CD8+ cytotoxic T cells (CTL).

In embodiments, the marker is or comprises a sialic acid-binding immunoglobulin-like lectin (Siglec) molecule. In embodiments, the marker is or comprises Siglec-1. In embodiments, the marker is one or more of CD2, CD3, CD4, CXCR4, CCR5, CD20, CD25, CD30, CD32a, CD69, CD91, CD160, CD257, LAG-3, CD147, CD231, CEACAM1, PLXNB2, HLA-DR, PD-1, CTLA-4, TIGIT, LAG-3, TIM-3, BTK, Cyclophilin B, Sec62, Rab10, SPCS, or a combination thereof.

In embodiments, the BioNV is derived from a modified cell, wherein the modified cell is a stem cell, an induced pluripotent stem cell (iPSC), a reprogrammed pluripotent or multipotent cell, an embryonic stem cell, a mesenchymal stem cell, or a differentiated cell which originates from any modified cell thereof. In embodiments, the modified cell is an iPSC. In embodiments, the modified cell is a T cell, helper T cell, T-memory cell, or NK cell.

In embodiments, the modified cell substantially lacks one or more MHC class I proteins, MHC class II proteins, T cell receptor (TCR) proteins, and/or cytokine release syndrome (CRS) proteins. In embodiments, the modified cell has reduced or ablated expression of a β2-macroglobulin (B2M) gene and/or reduced or ablated MHC class I protein expression and/or activity. In embodiments, the modified cell has reduced or ablated expression of a MHC II transactivator (CIITA) gene and/or reduced or ablated MHC class II protein expression and/or activity.

In embodiments, the modified cell has reduced or ablated expression of an HLA-A gene and/or reduced or ablated HLA-A protein expression and/or activity. In embodiments, the modified cell has reduced or ablated expression of an HLA-B gene and/or reduced or ablated HLA-B protein expression and/or activity. In embodiments, the modified cell has reduced or ablated expression of an HLA-C gene and/or reduced or ablated HLA-C protein expression and/or activity. In embodiments, the modified cell has reduced or ablated expression of an HLA-E or HLA-G gene and/or reduced or ablated HLA-E or HLA-G protein expression and/or activity. In embodiments, the modified cell has reduced or ablated expression of an HLA-F gene and/or reduced or ablated HLA-F protein expression and/or activity.

In embodiments, the modified cell has reduced or ablated expression of a T cell alpha constant (TRAC) gene and/or reduced or ablated TRAC protein expression and/or activity. In embodiments, the modified cell has reduced or ablated expression of a T cell beta constant (TRBC) gene and/or reduced or ablated TRBC protein expression and/or activity. In embodiments, the modified cell has reduced or ablated expression of a PD-1 gene and/or reduced or ablated PD-1 protein expression and/or activity, wherein the modified cell is activated; or the modified cell has expression or increased expression of a PD-1 gene and/or gene product, wherein the modified cell is not activated.

In embodiments, the modified cell has reduced or ablated expression of an IL-4 gene and/or reduced or ablated IL-4 protein expression and/or activity. In embodiments, the modified cell has reduced or ablated expression of an IL-6 gene and/or reduced or ablated IL-6 protein expression and/or activity. In embodiments, the modified cell has reduced or ablated expression of an IL-10 gene and/or reduced or ablated IL-10 protein expression and/or activity. In embodiments, the modified cell has reduced or ablated expression of an IL-16 gene and/or reduced or ablated IL-16 protein expression and/or activity.

In embodiments, the modified cell has reduced or ablated expression of a SerpinB9 gene and/or reduced or ablated SerpinB9 protein expression and/or activity. In embodiments, the modified cell expresses or has increased expression of a SerpinB9 gene and/or gene product.

In embodiments, the modified cell expresses or has increased expression of a CD34 gene and/or gene product. In embodiments, the modified cell expresses or has increased expression of a CCL2 gene and/or gene product. In embodiments, the modified cell expresses or has increased expression of a PD-L1 gene and/or gene product, and wherein the modified cell is not activated; or wherein the modified cell has reduced or ablated expression of a PD-L1 gene and/or gene product, and wherein the modified cell is activated. In embodiments, the modified cell expresses or has increased expression of a H2-M3 gene and/or gene product.

In embodiments, the modified cell expresses or has increased expression of a CD47 gene and/or gene product. In embodiments, the modified cell expresses or has increased expression of a CD24 gene and/or gene product. In embodiments, the modified cell expresses or has increased expression of a chimeric CD24/CD47 gene and/or gene product.

In embodiments, the modified cell expresses or has increased expression of a chimeric CD200 gene and/or gene product. In embodiments, the modified cell expresses or has increased expression of a chimeric CD24/CD200 gene and/or gene product or a chimeric CD47/CD200 gene and/or gene product.

In embodiments, the modified cell expresses or has increased expression of a chimeric CTLA-4 gene and/or gene product. In embodiments, the modified cell expresses or has increased expression of a chimeric MFG-E8 gene and/or gene product. In embodiments, the modified cell expresses or has increased expression of an NCAM gene and/or gene product. In embodiments, the modified cell expresses or has increased expression of a chimeric α-phagocytic integrin gene and/or gene product.

In embodiments, the modified cell expresses or has increased expression of a FasL gene and/or gene product. In embodiments, the modified cell wherein does not overexpress a FasL gene and/or gene product.

In embodiments, the modified cell has reduced or ablated expression and/or activity of 3 or more immunogenic proteins, 4 or more immunogenic proteins, 5 or more immunogenic proteins, 6 or more immunogenic proteins, 7 or more immunogenic proteins, 8 or more immunogenic proteins, 9 or more immunogenic proteins, 10 or more immunogenic proteins, 11 or more immunogenic proteins, or 12 or more immunogenic proteins. In embodiments, the modified cell expresses or has increased expression of 3 or more immunoprotective proteins, 4 or more immunoprotective proteins, 5 or more immunoprotective proteins, 6 or more immunoprotective proteins, 7 or more immunoprotective proteins, 8 or more immunoprotective proteins, 9 or more immunoprotective proteins, or 10 or more immunoprotective proteins.

In embodiments, the modified cell is allogeneic. In embodiments, the modified cell does not cause an immune reaction in patients to which it or a BioNV derived therefrom is administered.

In embodiments, the BioNV comprises one or more targeting agents directed to the marker on its surface. In embodiments, the one or more targeting agents is a chimeric antigen receptor (CAR), viral epitope recognition receptor (VERR), and/or viral ligand. In embodiments, the targeting agent is a ligand for a receptor or a receptor for a ligand, optionally wherein the VERR and/or viral ligand is a gp120/gp41 complex. In embodiments, the targeting agent is a ligand for a receptor or a receptor for a ligand, optionally wherein the VERR and/or viral ligand is a gp120/gp41 complex. In embodiments, the targeting agent comprises one or more of a transmembrane domain, an intracellular domain, a costimulatory domain, and/or a signaling domain. In embodiments, the targeting agent lacks an intracellular portion.

In embodiments, the targeting agent is a CAR. In embodiments, the CAR lacks an intracellular portion. In embodiments, the CAR comprises a viral epitope recognition receptors (VERR) or viral ligands comprising the targeting moiety. In embodiments, the transmembrane domain is derived from CD28, CD37, CD4, CD8a, or ICOS, or a fragment thereof. In embodiments, the intracellular domain comprises an intracellular signaling domain of a CD37-chain and/or one or more co-stimulatory molecules, optionally selected from CD28, 4-1BB, ICOS, CD27, and OX40.

In embodiments, the targeting agent is an antibody or an antibody format. In embodiments, the antibody or antibody format is selected from one or more of a CAR, VERR, viral ligand, Fab, Fab′, Fab′-SH, F(ab′)2, Fv, single chain Fv (scFv), diabody, nanobody, monoclonal antibody, polyclonal antibody, antibody fragment, linear antibody, bispecific antibody, multi-specific antibody, chimeric antibody, humanized antibody, human antibody, and fusion protein comprising the antigen-binding portion of an antibody. In embodiments, the antibody format is a CAR scFv and/or a VERR-scFv fusion.

In embodiments, the BioNV is about 10 nm to about 1200 nm in size. In embodiments, the BioNV is about 10 nm to about 100 nm in size. In embodiments, the BioNV is about 100 nm to about 200 nm in size. In embodiments, the BioNV is about 200 nm to about 500 nm in size. In embodiments, the BioNV is about 500 nm to about 1200 in size.

In embodiments, the BioNV substantially lacks expression and/or activity of HLA-A, HLA-B, HLA-C, HLA-F, CIITA, IL-6, TRAC, TRBC, and one of either HLA-E or HLA-G.

In embodiments, the BioNV substantially lacks expression and/or activity of HLA-A, HLA-B, HLA-C, HLA-F, CIITA, IL-6, TRAC, TRBC, SerpinB9, and one of either HLA-E or HLA-G.

In embodiments, the BioNV substantially lacks expression and/or activity of HLA-A, HLA-B, HLA-C, HLA-F, CIITA, IL-6, TRAC, TRBC, SerpinB9, CD200, one of either HLA-E or HLA-G, and one or more of IL-4, IL-10, and IL-16.

In embodiments, the BioNV comprises a membrane-embedded α-phagocytic integrin, CCL2, H2-M3, FasL, MFG-E8, and PD-L1 and/or CTLA-4, and one of either CD24, CD47, CD200, chimeric CD24/CD47, chimeric CD24/CD200, and chimeric CD47/CD200, or two of either CD24, CD47, and CD200.

In embodiments, the BioNV comprises a membrane-embedded α-phagocytic integrin, CCL2, H2-M3, FasL, MFG-E8, SerpinB9, and PD-L1 and/or CTLA-4, and one of either CD24, CD47, CD200, chimeric CD24/CD47, chimeric CD24/CD200, and chimeric CD47/CD200, or two of either CD24, CD47, and CD200.

In embodiments, the BioNV comprises a membrane-embedded CD200 protein and substantially lacks either CD24 or CD47. In embodiments, the BioNV substantially lacks protein and/or activity of SerpinB9 and CD200.

In embodiments, the checkpoint inhibiting agent is an agent that modulates one or more of PD-1, PD-L1, PD-L2, CTLA-4, TIM-3, LAG-3, B7-H3, B7-H4, A2aR, CD73, NKG2A, PVRIG/PVRL2, CEACAM1, CEACAM 5/6, FAK, CCL2/CCR2, LIF, CD47/SIRPα, CSF-1 (M-CSF)/CSF-1R, IL-1, IL-1R3, IL-8, SEMA4D, Ang-2, CLEVER-1, Axl, Phosphatidylserine. In embodiments, the checkpoint inhibiting agent is an antibody or an antibody format. In embodiments, the antibody or antibody format is specific for one of PD-1, PD-L1, PD-L2, CTLA-4, TIM-3, LAG-3, B7-H3, B7-H4, A2aR, CD73, NKG2A, PVRIG/PVRL2, CEACAM1, CEACAM 5/6, FAK, CCL2/CCR2, LIF, CD47/SIRPα, CSF-1 (M-CSF)/CSF-1R, IL-1, IL-1R3, IL-8, SEMA4D, Ang-2, CLEVER-1, Axl, Phosphatidylserine. In embodiments, the antibody or antibody format is selected from one or more of a monoclonal antibody, polyclonal antibody, antibody fragment, VERR, V_{NAR}, V_{H}H, Fab, Fab′, Fab′-SH, F(ab′)2, Fv, scFv, diabody, nanobody, linear antibody, bispecific antibody, multi-specific antibody, chimeric antibody, humanized antibody, human antibody, and fusion protein comprising the antigen-binding portion of an antibody.

In embodiments, the antibody or antibody format is specific for PD-1 and is selected from nivolumab, pembrolizumab, and pidilizumab. In embodiments, the antibody or antibody format is specific for PD-L1 and is selected from atezolizumab, avelumab, durvalumab, and BMS-936559. In embodiments, the antibody or antibody format is specific for CTLA-4 and is selected from ipilimumab, tremelimumab, AGEN1884, and RG2077.

In embodiments, the viral infection is a latent infection. In embodiments, the viral infection is a retroviral infection. In embodiments, the retroviral infection is a human immunodeficiency virus (HIV) infection. In embodiments, the HIV is HIV-1 or HIV-2. In embodiments, the HIV-1 is one of Group M, Group N, Group O, and Group P. In embodiments, the HIV-1 is Group M and a subtype selected from subtype A, B, C, D, F, G, H, I, J, K, and L. In embodiments, the retroviral infection is one of bovine immunodeficiency virus, caprine arthritis encephalitis virus, equine infectious anemia virus, feline immunodeficiency virus, Jembrana disease virus, puma lentivirus, simian immunodeficiency virus, visna-maedi virus, and human T-lymphotropic virus type 1 (HTLV-1) and HTLV-2.

In embodiments, the BioNV comprises one or more perforin molecules. In embodiments, the BioNV comprises one or more granzyme molecules. In embodiments, the granzyme molecules are selected from granzyme A, B, H, K, and M. In embodiments, the BioNV encapsulates one or more perforin and one or more granzyme molecules derived from a cell from which the BioNV is derived. In embodiments, the BioNV comprises one or more perforin and one or more granzyme molecules exogenously added to the BioNV.

In embodiments, the methods of treatment further comprise administering one or more antiretroviral therapies. In embodiments, the BioNV encapsulates one or more antiretroviral therapies. In embodiments, the antiretroviral therapy is a non-nucleoside reverse transcriptase inhibitor (NNRTI), optionally selected from efavirenz (SUSTIVA), rilpivirine (EDURANT) and doravirine (PIFELTRO). In embodiments, the antiretroviral therapy is a nucleoside or nucleotide reverse transcriptase inhibitor (NRTI), optionally selected from abacavir (ZIAGEN), tenofovir (VIREAD), emtricitabine (EMTRIVA), lamivudine (EPIVIR), zidovudine (RETROVIR), emtricitabine/tenofovir (TRUVADA), and emtricitabine/tenofovir alafenamide (DESCOVY). In embodiments, the antiretroviral therapy is a protease inhibitor (PI), optionally selected from atazanavir (REYATAZ), darunavir (PREZISTA), and lopinavir/ritonavir (KALETRA). In embodiments, the antiretroviral therapy is an integrase inhibitor, optionally selected from bictegravir sodium/emtricitabine/tenofovir alafenamide fumar (BIKTARVY), raltegravir (ISENTRESS), and dolutegravir (TIVICAY). In embodiments, the antiretroviral therapy is an entry or fusion inhibitor, optionally selected from enfuvirtide (FUZEON) and maraviroc (SELZENTRY). In embodiments, the antiretroviral therapy comprises at least two, or at least three, or at least four of the antiretroviral therapies in combination.

In embodiments, the BioNV comprises one or more gene editing payloads and one or more antiretroviral therapies.

In embodiments, methods of treatment further comprise co-administering a whole cell therapy. In embodiments, the methods of treatment further comprise administering an additional therapeutic agent.

In embodiments, the BioNV is stored at about −80 °C or is suitable for storage at about −80 °C. In embodiments, the BioNV is lyophilized.

In embodiments, the method causes the excision of viral nucleic acid from one or more infected cells, optionally HIV-infected cells. In embodiments, the method causes the excision of viral nucleic acid from one or more latently infected cells, optionally latently HIV-infected cells. In embodiments, the method causes a prevention of clonal expansion of infected cells, optionally HIV-infected cells. In embodiments, the method causes eradication, stoppage, halt or end of HIV or infection symptoms, or the progression of the symptoms or virus in the subject. In embodiments, the method induces and maintains sustained viral control, optionally selected from undetectable levels of plasma viremia and/or maintenance of less than about 50 copies/mL of HIV viral RNA, optionally as assayed by a polymerase chain reaction (PCR) test, a branched chain DNA (bDNA) test or a nucleic acid sequence-based amplification (NASBA) test.

In aspects, the present disclosure relates to compositions for the treatment of viral infection comprising an allogeneic, hypoimmunogenic biomimetic nanovesicle (BioNV) comprising (i) a membrane-embedded viral epitope recognition receptor (VERR) or viral ligand targeted against one or more markers of cellular infection with the virus, (ii) one or more membrane-embedded proteins of an α-phagocytic integrin, CCL2, H2-M3, FasL, MFG-E8, PD-L1 and/or CTLA-4, and SerpinB9, (iii) a membrane-embedded protein of one of either CD24, CD47, CD200, chimeric CD24/CD47, chimeric CD24/CD200, and chimeric CD47/CD200, or two of either CD24, CD47, and CD200, and (iv) a membrane substantially lacking proteins of one or more of HLA-A, HLA-B, HLA-C, HLA-F, CIITA, IL-6, TRAC, TRBC, one of either HLA-E or HLA-G, and one or more of IL-4, IL-10, and/or IL-16, and a checkpoint inhibitor.

In aspects, the present disclosure relates to compositions for the treatment of viral infection comprising an allogeneic, hypoimmunogenic biomimetic nanovesicle (BioNV) comprising (i) a membrane-embedded viral epitope recognition receptor (VERR) or viral ligand targeted against one or more markers of cellular infection with the virus, (ii) one or more membrane-embedded proteins of an α-phagocytic integrin, CCL2, H2-M3, FasL, MFG-E8, PD-L1 and/or CTLA-4, (iii) a membrane-embedded protein of either chimeric CD24/CD47, or CD24 and CD47, and (iv) a membrane substantially lacking proteins of one or more of HLA-A, HLA-B, HLA-C, HLA-F, CIITA, IL-6, TRAC, TRBC, SerpinB9, CD200, one of either HLA-E or HLA-G, and one or more of IL-4, IL-10, and/or IL-16, and a checkpoint inhibitor.

In embodiments, the BioNV encapsulates a gene editing payload. In embodiments, the gene editing payload comprises one or more gene editor nucleic acids and/or proteins, or one or more nucleic acids encoding one or more gene editors. In embodiments, the one or more gene editors is a site-directed endonuclease, TALEN, ZFN, RNase P RNA, CRISPR/Cas nuclease, C2c1, C2c2, C2c3, Cas9, Cpf1, TevCas9, Archaea Cas9, CasY.1, CasY.2, CasY.3, CasY.4, CasY.5, CasY.6, CasX, Cas omega, transposase, and/or any ortholog or homolog thereof. In embodiments, the gene editing payload comprises a transactivating response region (TAR) loop system.

### BRIEF DESCRIPTION OF THE DRAWINGS

FIG. 1 depicts a diagrammatic representation of a non-limiting exemplary workflow of modifying iPSCs for generating biomimetic nanovesicles (BioNVs).

FIG. 2 depicts a diagrammatic representation illustrating a non-limiting exemplary embodiment of the use of a CRISPR gene activation system (CRISPRa) regulated by a Tetracycline on/off promoter to activate upstream transcriptional activators. When expressed, the upstream transcriptional activators lead to overexpression of a downstream integrated cassette (containing CRISPR directed swap sites for any scFv, VERR and/or viral ligand, or VHH nanobody) strategically placed in an antibody locus (antibody locus swapped out).

FIG. 3 depicts a diagrammatic representation showing that BioNV manufacture can be performed from iPSCs prior to or after differentiation into lymphocyte cell lines.

FIG. 4 depicts a diagrammatic representation of a non-limiting illustrative embodiment of a hypoimmunogenic iPSC-derived BioNV.

FIG. 5 depicts a diagrammatic representation of two different VERR and/or viral ligand structures (primary and secondary generation) that can be expressed in iPSCs to generate the BioNVs.

FIGS. 6A-6C depicts a diagrammatic representation of select VERR and/or viral ligand construct designs. FIG. 6A illustrates a prototypical 2^{nd} generation VERR and/or viral ligand construct.

FIG. 6B is a representation of a 2^{nd} generation VERR and/or viral ligand construct with various combinations of VERRs or viral ligands. FIG. 6C is a representation of a VERR and/or viral ligand constructs with various combinations of single chain variable fragments (scFvs).

FIG. 7 depicts a non-limiting diagrammatic representation of the generation of BioNVs from iPSCs.

FIG. 8 depicts a graphic representation of the size distribution of BioNVs generated from iPSCs as measured by dynamic light scattering (DLS).

FIG. 9 depicts a diagrammatic representation of non-limiting illustrative examples of BioNVs that may be engineered, manufactured, and purified for methods of treatment described herein.

FIG. 10 depicts a diagrammatic representation of a non-limiting exemplary 2^{nd} generation VERR receptor that replaces the scFv (and linker), or variable heavy chain IgG fragment (VHH nanobody), or Immunoglobulin New Antigen Receptor (IgNAR) variable region called a VNAR with a VERR or viral ligand.

FIG. 11 depicts a diagrammatic representation of a non-limiting exemplary strategy for fusing a BioNV lipid bilayer with the plasma membrane of a targeted cell using an acidic activated fusion protein (PLA2) model.

FIG. 12 depicts a diagrammatic representation of a non-limiting exemplary strategy for fusing a BioNV lipid bilayer with the plasma membrane of a target cell using transmembrane anchored membrane fusion protein (or protein complex) fused with an acid-activatable PLA2 protein.

FIG. 13 depicts a diagrammatic representation of a non-limiting exemplary strategy for fusing a BioNV lipid bilayer with the plasma membrane of a target cell using a CAR-target or VERR-target-activated fusion protein model with PLA2 structurally linked the CAR, VERR, or viral ligand.

FIG. 14 depicts a diagrammatic representation of a non-limiting exemplary strategy for using HIV gp120/gp41 receptor ligand complexes expressed on the surface of a BioNV for the targeted fusion to a cell of interest; the deliverable payload is directly injected into the cytoplasm of the target cell, avoiding the endosomal pathway.

FIG. 15 depicts a diagrammatic representation of viral-ligand targeted BioNV with an engineered viral receptor.

FIGS. 16A-16B depicts a diagrammatic representation of a non-limiting exemplary BioNV with a bispecific CAR. FIG. 16A depicts a BioNV with only bispecific CARs. FIG. 16B depicts a BioNV with both monospecific CAR and a bispecific CAR.

FIG. 17 depicts a non-limiting diagrammatic representation of serial extrusion to produce BioNVs.

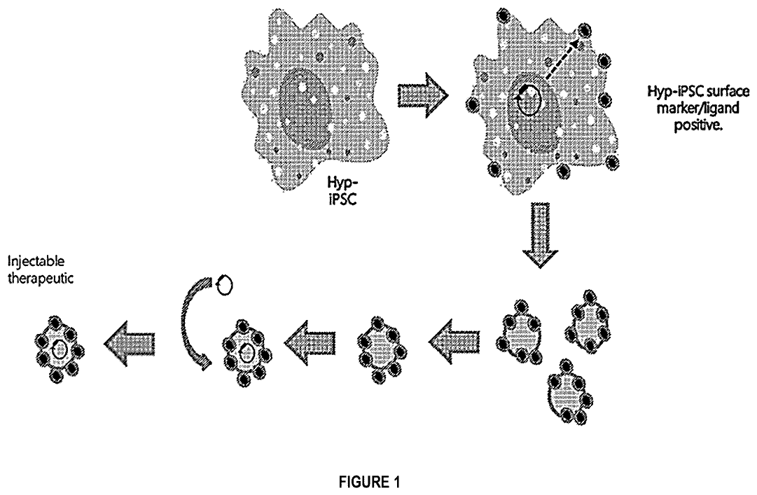

### DETAILED DESCRIPTION

The present disclosure relates to, in part, allogeneic, hypoimmunogenic modified cell-derived biomimetic nanovesicles (BioNVs) that find use in viral infections with surface-oriented viral epitope recognition receptors (VERRs) or viral ligands which recognize a single target or multiple targets through a binding moiety (e.g., single chain variable fragment (scFV)), for a desired/specific biomarker. This results in a designed BioNV on the order of 20-1200 nm in size, far smaller in size than a traditional cell-based T/NK cell therapies. The BioNV can originate form a modified stem cell, such as an induced pluripotent stem cell (iPSC). The modified cell (e.g., iPSC) plasma membrane-derived BioNVs retain hypoimmunogenic properties due to genetic engineering focused on knock-out of specific immunogenic cell surface markers (e.g., MHC I/II, TCR, CRS-related cytokines, etc.) and/or expression or increased expression of immunoprotective cell surface markers (e.g., CD47, CD34, CD24, CD200, α-phagocytic, etc.). The binding moiety of the VERR and/or viral ligand can comprise all variations of an antibody construct, including for example, Fab, Fab′, Fab′-SH, F(ab′)2, scFv, diabody, nanobody, linear antibody, bispecific antibody, multi-specific antibody, chimeric antibody, humanized antibody, human antibody, and fusion proteins comprising the antigen-binding portion of an antibody, VHH nanobodies, VNARS, among other antibody formats, allowing BioNVs to target of any cell of interest (targeting any type of cell surface biomarker). The BioNVs can also encapsulate and deliver any small molecule, biologic, nucleic acid, gene editing therapeutic payload, etc., of choice to the intended cellular targets to treat viral diseases.

Moreover, the present disclosure relates to, in part, methods of treatment, prevention, or ameliorating viral infection, especially in instances of retroviral infection or latent viral infection, using the present BioNV compositions. For example, in embodiments, the BioNV can be used in methods of treatment to target immune cell surface receptor, Siglec-1, in a subset of latently HIV-infected immune cell subsets in PLWH, regardless of ART or checkpoint inhibitor status. Those skilled in the art, upon review of this disclosure in its entirety, will appreciate that the methods of treatment using BioNVs can be tailored for treating and preventing viral infection, among other diseases and disorders, by administering an effective amount of the BioNV, which can deliver a variety of therapeutic payloads.

### Methods of Treatment or Prevention

In aspects, the present disclosure includes methods for treating, preventing, or ameliorating a viral infection, comprising administering to a subject in need thereof, a composition comprising a therapeutically effective amount of (a) a biomimetic nanovesicle (BioNV), the BioNV being targeted to one or more markers of cellular infection with the virus, and (b) one or more checkpoint inhibiting agents.

In aspects, the present disclosure includes methods for treating, preventing, or ameliorating a viral infection, comprising administering to a subject in need thereof, a composition comprising a therapeutically effective amount of a biomimetic nanovesicle (BioNV), the BioNV being targeted to one or more markers of cellular infection with the virus, wherein the subject is undergoing treatment with one or more checkpoint inhibiting agents.

In aspects, the present disclosure includes methods for treating, preventing, or ameliorating a viral infection, comprising administering to a subject in need thereof, a composition comprising a therapeutically effective amount of one or more checkpoint inhibiting agents, wherein the subject is undergoing treatment with a biomimetic nanovesicle (BioNV), the BioNV being targeted to one or more markers of cellular infection with the virus.

In aspects, the present disclosure includes methods for treating, preventing, or ameliorating a viral infection, comprising administering to a subject in need thereof a therapeutically effective amount of a biomimetic nanovesicle (BioNV), the BioNV: (i) being targeted to one or more markers of cellular infection with the virus; and (ii) comprising one or more checkpoint inhibiting agents.

In embodiments, the BioNV comprising the one or more checkpoint inhibiting agents can encapsulate the agents within an aqueous core and/or have the agents adhered to the surface (internal or external) of the BioNV. In embodiments, BioNVs can encapsulate at least one checkpoint inhibiting agent within the lumen of the NV. In embodiments, the BioNV can entrap the agent within the lipid layers of the NV. BioNVs can be multilamellar or unilamellar with the inner and/or outer leaflets of the plasma membrane of the cell from which it is derived.

In embodiments, the marker to be targeted is a cell surface receptor or cell surface ligand of a virally-infected cell. In embodiments, the marker is expressed on one or more of T cells, dendritic cells, and macrophages, or any cell type in an HIV-infected cell reservoir. The T cells can be CD4+ T cells or CD8+ cytotoxic T cells (CTLs). The marker can be expressed by immune cells, including any lymphoid progenitor lineage (e.g., T cells subsets such as Tregs, Th17, Th2, Th1, Th0, and Th22), or any myeloid progenitor lineage (e.g., mast cells, myoblast, monocytes, eosinophils, basophils, neutrophils, DCs, and macrophages).

In embodiments, the marker for methods herein is, or comprises, a sialic acid-binding immunoglobulin-like lectin (Siglec) molecule. In embodiments, the marker is or comprises Siglec-1. In embodiments, in the context of targeting latently HIV-infected immune cells, sialic acid-binding immunoglobulin-like lectins (Siglecs), such as Siglec-1, can be the effective target for the methods of treatment. Siglec targets can be divided into subsets based on sequence and structure similarity, such as CD33-related Siglecs (e.g., Siglec-H, Siglec-5, and Siglec-14) and CD22-related Siglecs.

In embodiments, the marker for methods herein is, or comprises, CD32a (also known as FcγRIIa). In embodiments, methods targeting CD32a cell surface expression of the low affinity Fc receptor CD32a allow targeting of the replication-competent HIV-1 reservoir in CD4+ T cells of HIV-1-infected participants receiving suppressive anti-retroviral therapy (ART). In embodiments, methods targeting CD32a biomarker are used to deliver a payload, e.g., a gene editor to non-infected cells. In embodiments, CD32α-targeted BioNVs carry a gene editing payload which is not active in non-virally infected cells. In embodiments, the BioNV is targeted against at least a portion of CD32a (surface receptor glycoprotein also known as FcγRIIa). In embodiments, methods herein target HIV infected cells and/or reservoirs of HIV proviruses in cells that express CD32a. CD32a is a cell surface marker whose expression has been associated with latent HIV-1 infection (BELSHAN et al. 2021) and (DESCOURS et al., “CD32a is a marker of a CD4 T-cell HIV reservoir harbouring replication-competent proviruses,” Nature, Vol. 543, 2017:564-7). In embodiments, the BioNV is targeted against at least a portion of CD32a and at least one other cell surface marker (e.g., via a bispecific CAR, multiple VERRs, viral ligands, and/or viral receptors).

In embodiments, the marker for methods herein is, or comprises, HLA-DR. In embodiments, methods targeting HLA-DR allow targeting of the replication-competent HIV-1 reservoir in participants receiving suppressive anti-retroviral therapy (ART). In embodiments, methods targeting a HLA-DR biomarker are used to deliver a payload, e.g., a gene editor to non-infected cells. In embodiments, HLA-DR-targeted BioNVs carry a gene editing payload which is not active in non-virally infected cells. In embodiments, methods herein target HIV infected cells and/or reservoirs of HIV proviruses in cells that express a canonical marker of activation, HLA-DR (MHC class II). HLA-DR expression has been shown to be associated with cells latently harboring HIV proviral sequences, for example as described in (COHN L B, et al., “The Biology of the HIV-1 Latent Reservoir and Implications for Cure Strategies,” Cell Host Microbe, Vol. 27, No. 4, 2020: pp. 519-30), (HORSBURGH B A, et al., “High levels of genetically intact HIV in HLA-DR+ memory T cells indicates their value for reservoir studies,” AIDS. Vol. 34, No. 5, 2020:659-668) and (LEE E, et al., “Memory CD4+ T-Cells Expressing HLA-DR Contribute to HIV Persistence During Prolonged Antiretroviral Therapy,” Front Microbiol, Vol. 10, No. 2214, 2019: pp. 1-19). In embodiments, the BioNV is targeted against at least a portion of HLA-DR and at least one other cell surface marker (e.g., via a bispecific CAR, multiple VERRs, viral ligands, and/or viral receptors).

In embodiments, the marker for methods herein is, or comprises, CD25 (the alpha chain of the IL-2 receptor and a constitutive marker of regulatory T cells). In embodiments, methods targeting CD25 allow targeting of the replication-competent HIV-1 reservoir in participants receiving suppressive anti-retroviral therapy (ART). In embodiments, methods targeting a CD25 biomarker are used to deliver a payload, e.g., a gene editor to non-infected cells. In embodiments, CD25-targeted BioNVs carry a gene editing payload which is not active in non-virally infected cells. In embodiments, methods herein target HIV infected cells and/or reservoirs of HIV proviruses in cells that express CD25. CD25 expression has been shown to be associated with cells latently harboring HIV proviral sequences, for example as described in (COHN, et al. 2020) and (TRAN T A, et al., “Resting regulatory CD4 T cells: a site of HIV persistence in patients on long-term effective antiretroviral therapy,” PLoS One. Vol. 3, No. 10, 2008: pp. 1-11). In embodiments, the BioNV is targeted against at least a portion of CD25 and at least one other cell surface marker (e.g., via a bispecific CAR, multiple VERRs, viral ligands, and/or viral receptors).

In embodiments, the marker for methods herein is, or comprises, CD69 (a marker of tissue-resident memory T cells). In embodiments, methods targeting CD69 allow targeting of the replication-competent HIV-1 reservoir in participants receiving suppressive anti-retroviral therapy (ART). In embodiments, methods targeting a CD69 biomarker are used to deliver a payload, e.g., a gene editor to non-infected cells. In embodiments, CD69-targeted BioNVs carry a gene editing payload which is not active in non-virally infected cells. In embodiments, methods herein target HIV infected cells and/or reservoirs of HIV proviruses in cells that express CD69. CD69 expression has been shown to be associated with cells latently harboring HIV proviral sequences, for example as described in (COHN, et al. 2020) and (CANTERO-PÉREZ J, et al., “Resident memory T cells are a cellular reservoir for HIV in the cervical mucosa,” Nat Commun. Vol. 10, No. 1, 2019: pp. 1-16). In embodiments, the BioNV is targeted against at least a portion of CD69 and at least one other cell surface marker (e.g., via a bispecific CAR, multiple VERRs, viral ligands, and/or viral receptors).

Methods herein, in embodiments, target subsets of cells that express HLA-DR, CD25, and/or CD69. Methods herein, in embodiments, target subsets of cells that do not fulfill the classical definition of resting CD4+ T cells which can contribute to the long-term persistence of HIV during ART.

In embodiments, the marker for methods herein is, or comprises, one or more of programmed cell death-1 (PD-1), cytotoxic T-lymphocyte-associated protein 4 (CTLA-4), T cell immunoglobulin and ITIM domain (TIGIT), lymphocyte activation gene 3 (LAG-3), T cell immunoglobulin and mucin 3 (TIM-3) and/or CD160. In embodiments, methods targeting one or more of PD-1, CTLA-4, TIGIT, LAG-3, TIM-3 and/or CD160 allow targeting of the replication-competent HIV-1 reservoir in participants receiving suppressive anti-retroviral therapy (ART). In embodiments, methods target one or more of a PD-1, CTLA-4, TIGIT, LAG-3, TIM-3 and/or CD160 biomarker to deliver a payload, e.g., a gene editor to non-infected cells. In embodiments, PD-1, CTLA-4, TIGIT, LAG-3, TIM-3 and/or CD160-targeted BioNVs carry a gene editing payload which is not active in non-virally infected cells. In embodiments, methods herein target HIV infected cells and/or reservoirs of HIV proviruses in cells that express PD-1, CTLA-4, TIGIT, LAG-3, TIM-3, and/or CD160. PD-1, CTLA-4, TIGIT, LAG-3, TIM-3, and/or CD160 expression has been shown to be associated with cells latently harboring HIV proviral sequences, for example as described in (COHN, et al. 2020), (CHEW G M, et al., “TIGIT Marks Exhausted T Cells, Correlates with Disease Progression, and Serves as a Target for Immune Restoration in HIV and SIV Infection,” PLoS Pathog, Vol. 12, No. 1, 2016:1-28), (FROMENTIN R, et al., “CD4+ T Cells Expressing PD-1, TIGIT and LAG-3 Contribute to HIV Persistence during ART,” PLoS Pathog, Vol. 12, No. 7, 2016:1-19), and (PARDONS M, et al., “Single-cell characterization and quantification of translation-competent viral reservoirs in treated and untreated HIV infection,” PLoS Pathog, Vol. 15, No. 2, 2019: pp. 1-28). In embodiments, the BioNV is targeted against at least a portion of PD-1, CTLA-4, TIGIT, LAG-3, TIM-3, and/or CD160 and at least one other cell surface marker (e.g., via a bispecific CAR, multiple VERRs, viral ligands, and/or viral receptors).

In embodiments, the marker for methods herein is, or comprises, Cyclophilin B (CypB, also known as peptidyl prolyl isomerase B (PPIB), is a member of the cyclophilin family of immunophilins, which a cytosolic enzyme that can be enriched on the plasma membrane). In embodiments, methods targeting CypB allow targeting of the replication-competent HIV-1 reservoir in participants receiving suppressive anti-retroviral therapy (ART). In embodiments, methods targeting a CypB biomarker are used to deliver a payload, e.g., a gene editor to non-infected cells. In embodiments, CypB-targeted BioNVs carry a gene editing payload which is not active in non-virally infected cells. In embodiments, methods herein target HIV infected cells and/or reservoirs of HIV proviruses in cells that express CypB. CypB expression has been shown to upregulated during HIV replication, for example as described in (BELSHAN M, et al., “Discovery of candidate HIV-1 latency biomarkers using an OMICs approach,” Virology, Vol. 558, 2021: pp. 86-95), (DEBOER J, et al., “Alterations in the nuclear proteome of HIV-1 infected T-cells,” Virology, 468-470, 2014: pp. 409-420), and (DEBOER J, et al., “Cyclophilin B enhances HIV-1 infection,” Virology, Vol. 489, 2016: pp. 282-91). In embodiments, the BioNV is targeted against at least a portion of CypB and at least one other cell surface marker (e.g., via a bispecific CAR, multiple VERRs, viral ligands, and/or viral receptors).

In embodiments, the marker for methods herein is, or comprises, Sec62 (a component of the SEC61 complex that functions in protein translocation in the endoplasmic reticulum). In embodiments, methods targeting Sec62 allow targeting of the replication-competent HIV-1 reservoir in participants receiving suppressive anti-retroviral therapy (ART). In embodiments, methods targeting a Sec62 biomarker are used to deliver a payload, e.g., a gene editor to non-infected cells. In embodiments, Sec62-targeted BioNVs carry a gene editing payload which is not active in non-virally infected cells. In embodiments, methods herein target HIV infected cells and/or reservoirs of HIV proviruses in cells that express Sec62. Sec62 expression has been shown to be upregulated during HIV replication and to interact with HIV-1 gp41, for example as described in (BELSHAN, et al. 2021) and (JÄGER S, et al., “Global landscape of HIV-human protein complexes,” Nature, Vol. 481, 2012: pp. 365-370). In embodiments, the BioNV is targeted against at least a portion of Sec62 and at least one other cell surface marker (e.g., via a bispecific CAR, multiple VERRs, viral ligands, and/or viral receptors).

In embodiments, the marker for methods herein is, or comprises, Rab10 (a member of the RAS superfamily of small GTPases involved in vesicular transport). In embodiments, methods targeting Rab10 allow targeting of the replication-competent HIV-1 reservoir in participants receiving suppressive anti-retroviral therapy (ART). In embodiments, methods targeting a Rab10 biomarker are used to deliver a payload, e.g., a gene editor to non-infected cells. In embodiments, Rab10-targeted BioNVs carry a gene editing payload which is not active in non-virally infected cells. In embodiments, methods herein target HIV infected cells and/or reservoirs of HIV proviruses in cells that express Rab10. Rab10 overexpression is consistent with its presence in Staufen1-containing ribonucleoprotein complexes found in HIV-1 infected cells, for example as described in (BELSHAN, et al. 2021) and (MILEV M P, et al., “Characterization of staufen1 ribonucleoproteins by mass spectrometry and biochemical analyses reveal the presence of diverse host proteins associated with human immunodeficiency virus type 1,” Front Microbiol. Vol. 3, No. 367, 2012:1-21). In embodiments, the BioNV is targeted against at least a portion of Rab10 and at least one other cell surface marker (e.g., via a bispecific CAR, multiple VERRs, viral ligands, and/or viral receptors).

In embodiments, the marker for methods herein is, or comprises, signal peptidase complex subunit 1 (SPCS1) (SPCS1 is a component of the microsomal signal peptidase complex that participates in protein processing and transport in the endoplasmic reticulum). In embodiments, methods targeting SPCS1 allow targeting of the replication-competent HIV-1 reservoir in participants receiving suppressive anti-retroviral therapy (ART). In embodiments, methods targeting a SPCS1 biomarker are used to deliver a payload, e.g., a gene editor to non-infected cells. In embodiments, SPCS1-targeted BioNVs carry a gene editing payload which is not active in non-virally infected cells. In embodiments, methods herein target HIV infected cells and/or reservoirs of HIV proviruses in cells that express SPCS1. SPCS1 is involved in the assembly and budding of flaviviruses and its cellular expressed has been associated with HIV-1 infection (BELSHAN et al. 2021). In embodiments, the BioNV is targeted against at least a portion of SPCS1 and at least one other cell surface marker (e.g., via a bispecific CAR, multiple VERRs, viral ligands, and/or viral receptors).

In embodiments, the marker for methods herein is, or comprises, Bruton tyrosine kinase (BTK) (BTK is a tyrosine kinase involved in B cell development). In embodiments, methods targeting BTK allow targeting of the replication-competent HIV-1 reservoir in participants receiving suppressive anti-retroviral therapy (ART). In embodiments, methods targeting a BTK biomarker are used to deliver a payload, e.g., a gene editor to non-infected cells. In embodiments, BTK-targeted BioNVs carry a gene editing payload which is not active in non-virally infected cells. In embodiments, methods herein target HIV infected cells and/or reservoirs of HIV proviruses in cells that express BTK. BTK is a cell surface marker whose expression has been associated with latent HIV-1 infection (BELSHAN et al. 2021) and (BERRO et al., “Identifying the membrane proteome of HIV-1 latently infected cells,” J. Biol. Chem., Vol. 282, No. 11, 2007:8207-18). In embodiments, the BioNV is targeted against at least a portion of BTK and at least one other cell surface marker (e.g., via a bispecific CAR, multiple VERRs, viral ligands, and/or viral receptors).

In embodiments, the marker for methods herein is, or comprises, CD3 (T cell surface receptor complex). In embodiments, methods targeting CD3 allow targeting of the replication-competent HIV-1 reservoir in participants receiving suppressive anti-retroviral therapy (ART). In embodiments, methods targeting a CD3 biomarker are used to deliver a payload, e.g., a gene editor to non-infected cells. In embodiments, CD3-targeted BioNVs carry a gene editing payload which is not active in non-virally infected cells. In embodiments, methods herein target HIV infected cells and/or reservoirs of HIV proviruses in cells that express CD3. CD3 is a cell surface marker whose expression has been associated with latent HIV-1 infection (BELSHAN et al. 2021) and (IGLESIAS-USSEL, et al., “High levels of CD2 expression identify HIV-1 latently infected resting memory CD4+ T cells in virally suppressed subjects,” J Virol, Vol. 87, No. 16, 2013: pp. 9148-58). In embodiments, the BioNV is targeted against at least a portion of CD3 and at least one other cell surface marker (e.g., via a bispecific CAR, multiple VERRs, viral ligands, and/or viral receptors).

In embodiments, the marker for methods herein is, or comprises, CD2 (T cell and NK cell surface receptor complex). In embodiments, methods targeting CD2 allow targeting of the replication-competent HIV-1 reservoir in participants receiving suppressive anti-retroviral therapy (ART). In embodiments, methods targeting a CD2 biomarker are used to deliver a payload, e.g., a gene editor to non-infected cells. In embodiments, CD2-targeted BioNVs carry a gene editing payload which is not active in non-virally infected cells. In embodiments, methods herein target HIV infected cells and/or reservoirs of HIV proviruses in cells that express CD2, CD2 is a cell surface marker whose expression has been associated with latent HIV-1 infection (DARCIS, et al., “The Quest for Cellular Markers of HIV Reservoirs: Any Color You Like,” Front Immunol, Vol. 10, No. 2251, 2019: pp. 1-9) and (IGLESIAS-USSEL, et al., “High levels of CD2 expression identify HIV-1 latently infected resting memory CD4+ T cells in virally suppressed subjects,” J Virol, Vol. 87, No. 16, 2013: pp. 9148-58). In embodiments, the BioNV is targeted against at least a portion of CD2 and at least one other cell surface marker (e.g., via a bispecific CAR, multiple VERRs, viral ligands, and/or viral receptors).

In embodiments, the marker for methods herein is, or comprises, CD20 (B cell surface receptor complex). In embodiments, methods targeting CD20 allow targeting of the replication-competent HIV-1 reservoir in participants receiving suppressive anti-retroviral therapy (ART). In embodiments, methods targeting a CD20 biomarker are used to deliver a payload, e.g., a gene editor to non-infected cells. In embodiments, CD20-targeted BioNVs carry a gene editing payload which is not active in non-virally infected cells. In embodiments, methods herein target HIV infected cells and/or reservoirs of HIV proviruses in cells that express CD20. CD20 is a cell surface marker whose expression has been associated with HIV RNA⁺ cells (DARCIS et al. 2019) and (SERRA-PEINADO et al., “Expression of CD20 after viral reactivation renders HIV-reservoir cells susceptible to Rituximab,” Nat Commun, Vol. 10, No. 3705, 2019: pp. 1-15). In embodiments, the BioNV is targeted against at least a portion of CD20 and at least one other cell surface marker (e.g., via a bispecific CAR, multiple VERRs, viral ligands, and/or viral receptors).

In embodiments, the marker for methods herein is, or comprises, CD30 (overexpression associated with lymphomas; CD30 expression is triggered by viral infection). In embodiments, methods targeting CD30 allow targeting of the replication-competent HIV-1 reservoir in participants receiving suppressive anti-retroviral therapy (ART). In embodiments, methods targeting a CD30 biomarker are used to deliver a payload, e.g., a gene editor to non-infected cells. In embodiments, CD30-targeted BioNVs carry a gene editing payload which is not active in non-virally infected cells. In embodiments, methods herein target HIV infected cells and/or reservoirs of HIV proviruses in cells that express CD30. CD30 is a cell surface marker whose expression has been associated with HIV-infected CD4+ T cells (DARCIS et al. 2019), (BISWAS P, et al., “CD30 ligation differentially affects CXCR4-dependent HIV-1 replication and soluble CD30 secretion in non-Hodgkin cell lines and in gamma delta T lymphocytes,” Eur J Immunol. Vol. 33, No. 11, 2003: pp. 3136-45), (ROMAGNANI S, et al., “Role for CD30 in HIV expression,” Immunol Lett. Vol. 51, No. 1-2, 1996: pp. 83-8), (HOGAN L E, et al., “Increased HIV-1 transcriptional activity and infectious burden in peripheral blood and gut-associated CD4+ T cells expressing CD30,” PLoS Pathog, Vol. 14, No. 2, 2018: pp. 1-19), and (WANG C C, et al., “Transient loss of detectable HIV-1 RNA following brentuximab vedotin anti-CD30 therapy for Hodgkin lymphoma,” Blood Adv, Vol. 2, No. 23, 2018: pp. 3479-82). In embodiments, the BioNV is targeted against at least a portion of CD30 and at least one other cell surface marker (e.g., via a bispecific CAR, multiple VERRs, viral ligands, and/or viral receptors).

In embodiments, the marker to be targeted is one or more of CD2, CD3, CD4, CXCR4, CCR5, CD20, CD25, CD30, CD32a, CD69, CD91, CD160, CD257, LAG-3, CD147, CD231, CEACAM1, PLXNB2, HLA-DR, PD-1, CTLA-4, TIGIT, LAG-3, TIM-3, BTK, Cyclophilin B, Sec62, Rab10, SPCS, or a combination thereof. In embodiments, the BioNV comprises a bispecific chimeric receptor, such as, without limitation, one or more antibody or antibody formats described herein, for targeted two or more of Siglec-1, CD2, CD3, CD4, CXCR4, CCR5, CD20, CD25, CD30, CD32a, CD69, CD91, CD160, CD257, LAG-3, CD147, CD231, CEACAM1, PLXNB2, HLA-DR, PD-1, CTLA-4, TIGIT, LAG-3, TIM-3, BTK, Cyclophilin B, Sec62, Rab10, and/or SPCS. In embodiments, the BioNV comprises a bispecific chimeric receptor that targets a Siglec and CD32a.

BioNVs used in methods herein are, in embodiments, derived from a modified cell, such as a stem cell, an induced pluripotent stem cell (iPSC), a reprogrammed pluripotent or multipotent cell, an embryonic stem cell, a mesenchymal stem cell, or a differentiated cell which originates from any modified cell thereof. In embodiments, the modified cell is an iPSC. In embodiments, the iPSC is differentiated into a particular cell. In embodiments, wherein the modified cell is a T cell, helper T cell, T-memory cell, or NK cell, etc. In embodiments, the differentiated cell is a myeloid lineage cell, such as a macrophage, monocyte, neutrophil, etc. In embodiments, the differentiated cell is a tissue-specific cell, such as a hepatocyte, cardiomyocyte, neuron, endothelial cell, pancreatic cell, retinal pigmented epithelium (RPE) cell, etc. In embodiments, the hypoimmunogenic cell can be any terminally differentiated cell, for example and without limitation, a muscle cell (satellite cell), adipocyte, osteocyte, cardiomyocyte, hepatocyte, blood cell (including erythrocyte, thrombocyte, and all immune cell types), glial cell (among other neuronal cell types), epithelial cell, epidermal cell, interstitial cell (e.g., respiratory interstitial cell), fibroblast (e.g., dermal fibroblast), endothelial cell (e.g., bronchial endothelial cell), oral cell, stromal cell, or germ cell. In embodiments, the hypoimmunogenic cell can be any function-specific cell type, for example and without limitation, exocrine secretory epithelial cell, hormone-secreting cell (e.g. enteroendocrine cell, thyroid cell, pancreatic islet cell, etc.), sensory transducer cell, autonomic neuronal cell, sensory organ cell (e.g., pillar cell, olfactory cell, Schwann cell, satellite glial cell, etc.), barrier cell (e.g., pneumocyte, duct cell, kidney cell, podocyte, etc.), extracellular matrix cell (e.g., tendon fibroblast, osteoblast, connective tissue cell, etc.), or contractile cell (e.g., skeletal muscle cell, cardiac muscle cell, myoepithelial cell, etc.).

In embodiments, the modified cell is differentiated to express or not express a variety of cell surface markers. In embodiments, the methods include BioNVs derived from a hypoimmunogenic iPSC.

In embodiments, the modified cell (e.g., derived from iPSCs) is created by knocking-out, silencing, inactivating, blocking or otherwise negating the expression, transcriptional efficiencies, and/or activity of one or more immunogenic molecules. In embodiments, the modified cell substantially lacks one or more MHC class I proteins, MHC class II proteins, HLA proteins, TCR proteins, and/or CRS proteins.

In embodiments, the reducing or ablating the expression and/or activity of one or more immunogenic proteins includes a β2-macroglobulin (B2M) gene disruption and/or a disruption that reduces or ablates MHC class I protein expression and/or activity, such as in the case for CD8+ T cell lineages. In embodiments, the reducing or ablating the expression and/or activity of one or more immunogenic proteins includes a CIITA gene disruption and/or a disruption that reduces or ablates MHC class II protein expression and/or activity, such as in the case of CD4+ T cell lineages. Without wishing to be bound by theory, these proteins contribute to the human leukocyte antigen (HLA) immunogenicity that requires HLA allele matching in the donor-recipient for treatment by cell-based therapies. In embodiments, allogeneic and/or hypoimmunogenic properties are achieved by reducing or ablating the expression and/or activity of the genes encoding the T cell receptor (TCR) proteins including, for example, the α and β chains (as in the case of αβ T cells) or the γ and δ chains (as in the case of γδ T cells) forming the ligand-binding site and the signaling modules CD3δ, CD3γ, CD3ε, and CD3ζ. In embodiments, this is performed to reduce extraneous T cell receptor types other than those of the CAR and/or VERR and/or viral ligand cassette and further improve the homogeneity of the CAR and/or VERR and/or viral ligand of interest and reduce off-target effects in BioNV formation.

In embodiments, the modified cell includes a β2-macroglobulin (B2M) gene disruption, or a gene disruption that disrupts MHC class I expression. In embodiments, knocking-out of the B2M genes reduces the number of potential doses to be administered due to the risk of preventing long term acceptance of the BioNVs by the recipient, such as what has been observed in the whole cell-based approaches described above. To overcome this issue, in embodiments, the HLA-E or HLA-G gene remains intact, allowing the immune system to adapt to the resulting BioNV. In embodiments, the HLA-A, HLA-B, HLA-C, HLA-F, HLA-E or HLA-G (but not both) are knocked out sequentially.

In embodiments, the modified cell has reduced or ablated expression of an HLA-A gene and/or reduced or ablated HLA-A protein expression and/or activity. In embodiments, the modified cell has reduced or ablated expression of an HLA-B gene and/or reduced or ablated HLA-B protein expression and/or activity. In embodiments, the modified cell has reduced or ablated expression of an HLA-C gene and/or reduced or ablated HLA-C protein expression and/or activity. In embodiments, wherein the modified cell has reduced or ablated expression of an HLA-E or HLA-G gene and/or reduced or ablated HLA-E or HLA-G protein expression and/or activity. In embodiments, the modified cell has reduced or ablated expression of an HLA-F gene and/or reduced or ablated HLA-F protein expression and/or activity.

In embodiments, the modified cell has reduced or ablated expression of a CIITA gene and/or reduced or ablated MHC class II protein expression and/or activity. In embodiments, allogeneic iPSCs have their MHC Class I and MHC Class II complexes disrupted by knocking out critical proteins involved in their expression, for example, β2-macroglobulin (B2M) which is a serum protein found in association with the MHC class I heavy chain on the surface of nearly all nucleated cells that is involved in the presentation of peptide antigens to the immune system. In embodiments, the allogeneic iPSCs have their CIITA gene disrupted, which is the Master Control Transcription Factor that regulates the expression of all MHC II genes. In embodiments, allogeneic iPSCs have their CIITA gene disrupted, which is the master control transcription factor that regulates the expression of all MHC II genes so that a resulting differentiated cell line (e.g., DCs, mononuclear phagocytes, endothelial cells, thymic epithelial cells, B cells, etc.) does not express or has reduced expression of any MHC class II proteins.

In embodiments, the modified cell has reduced or ablated expression of a T cell alpha constant (TRAC) gene and/or reduced or ablated TRAC protein expression and/or activity. In embodiments, the modified cell has reduced or ablated expression of a T cell beta constant (TRBC) gene and/or reduced or ablated TRBC protein expression and/or activity. In embodiments, the modified cell has reduced or ablated expression of a PD-1 gene and/or reduced or ablated PD-1 protein expression and/or activity, wherein the modified cell is activated; or the modified cell has expression or increased expression of a PD-1 gene and/or gene product, wherein the modified cell is not activated.

CRS is a major concern with whole cell therapies, where despite engineered hypoimmunogenicity, effector functions and other consequences of interaction with cells post-infusion can result in the release of biomolecules that result in a systemic inflammatory syndrome characterized by fever, multiple organ dysfunction, etc. In embodiments, the modified cell is engineered to disrupt one or more proteins that contribute to CRS. In embodiments, the modified cell has reduced or ablated expression and/or activity (e.g., knock-out or silencing) of CRS-related cytokines.

In embodiments, the modified cell has reduced or ablated expression of an IL-4 gene and/or reduced or ablated IL-4 protein expression and/or activity. In embodiments, the modified cell has reduced or ablated expression of an IL-6 gene and/or reduced or ablated IL-6 protein expression and/or activity. In embodiments, IL-6 knock-out prevents undesirable IL-6 packaging into the BioNV and reduces the BioNV's contribution to a localized (and concentrated due to biomarker targeting) and/or potentially systemic CRS events. In embodiments, the modified cell has reduced or ablated expression of an IL-10 gene and/or reduced or ablated IL-10 protein expression and/or activity. In embodiments, the modified cell has reduced or ablated expression of an IL-16 gene and/or reduced or ablated IL-16 protein expression and/or activity. In embodiments, the reduction of, or ablation of, interleukins causing CRS decreases the likelihood of CRS.

Serine proteinase inhibitor B9 (SerpinB9) is a member of the serine protease inhibitor superfamily. SerpinB9 has been reported to protect cells from the immune-killing effects of granzyme B. In embodiments, such as where the BioNV is not designed to delivery granzyme, the modified cell that the BioNV is derived from expresses or has increased expression of SerpinB9. In embodiments, such as where the BioNV is designed to deliver granzyme, the modified cell that the BioNV is derived from has SerpinB9 knocked-out and/or silenced. In embodiments, the modified cell has reduced or ablated expression and/or activity of a SerpinB9 gene and/or reduced or ablated SerpinB9 protein expression and/or activity.

In embodiments, the overexpression of SerpinB9 sequesters the function of granzyme B which is related to immunostimulatory responses, such as apoptosis of a targeted and/or diseased cell. In embodiments, granzyme B is inhibited in activated lymphocytes, NK cells, macrophages, and follicular DCs, among other cell types. In embodiments, e.g., involving a BioNV that is intended to deliver a non-granzyme payload, for example a gene editing payload, the modified cell can express and/or have increased expression of SerpinB9.

In embodiments, methods of treating, preventing, and/or ameliorating viral infection use BioNVs which originate from cells modified to be hypoimmunogenic due to expression or increased expression of one or more immunoprotective proteins. In embodiments, the modified cell expresses or has increased expression of a CD34 gene and/or gene product. In embodiments, the modified cell expresses or has increased expression of a CCL2 gene and/or gene product. In embodiments, the modified cell expresses or has increased expression of a PD-L1 gene and/or gene product, and wherein the modified cell is not activated; or wherein the modified cell has reduced or ablated expression of a PD-L1 gene and/or gene product, and wherein the modified cell is activated. In embodiments, the modified cell expresses or has increased expression of a H2-M3 gene and/or gene product.

In embodiments, the modified cell expresses or has increased expression of a CD47 gene and/or gene product. Exosomes and cell-derived vesicles (CDVs) are readily cleared from the body by macrophages through phagocytosis. Phagocytosis greatly impacts the therapeutic value and efficacy of CDVs. To prevent macrophage depletion of BioNVs, but without wishing to be bound by theory, in embodiments, the BioNV is CD47 tagged on the surface. A CD47tg (tag) provides a “do not eat me” signal which, in embodiments, increases the half-life and serum stability of the BioNV in the subject. In embodiments, the molecular CD47 isoform 2 (the isoform that interacts with the SIRPα receptor on a macrophage) is engineered into the modified cell (e.g., iPSC cell line). Without the CD47tg, the BioNV half-life would be diminished due to phagocytosis inhibition, resulting in the need for higher and/or more frequent doses. In embodiments, prevention of the potential inhibitory phenotypes of CD47 expression across cells is done via interference with the inhibitory mechanism of action of the series of microRNAs on the 3′UTR of the CD47 gene by deleting this region in stable constructs or by eliminating/inhibiting the expression of the microRNAs. In embodiments, this can resolve inhibitory issues caused by the microRNAs across differentiated cell subsets.

In embodiments, the modified cell overexpresses CD24. CD24 is a asialoglycoprotein expressed on mature granulocytes and B-cells and is also an anti-phagocytic protein. CD24 prevents phagocytosis through interactions with Siglec-G/10 on macrophages. In embodiments, the modified cell expresses or has increased expression of a CD24 gene and/or gene product. In embodiments, the modified cell expresses or has increased expression of a chimeric CD24/CD47 gene and/or gene product. In embodiments, the modified cell expresses chimeric CD24/CD47 with a tethered transmembrane domain. In embodiments, the domains of CD47 isoform 2 and CD24 can be either separately expressed or tethered to form a bilobed, chimeric protein. In embodiments, the modified cells are iPSCs are from fibroblasts, not ABO cells.

In embodiments, the modified cell expresses or has increased expression of a chimeric CD200 gene and/or gene product. In embodiments, CD200 tags minimize phagocytosis by macrophages and also prevent the activation of granulocytes. In embodiments, the modified cell does not express CD200 when it is not desirable to prevent granulocytes, for example in the TME, as activation of granulocytes would complement the mechanism of action of a BioNV designed to release granzymes and perforins. However, in embodiments, if a CD47 or CD24 tag is used, or a CD24/CD47 chimeric, bilobed protein tag (each prevents phagocytosis) in combination with overexpressed H2-M3 (which dampens the NK response) is used, stability can be achieved without CD200, while allowing adequate BioNV clearance. In embodiments, where granzymes and perforins are not a therapeutic biomolecule of choice, CD200 can be expressed to prevent the activation of granulocytes, while eliminating a CD47 tag or a CD24 tag, but not both tags. In embodiments, the modified cell expresses or has increased expression of a chimeric CD24/CD200 gene and/or gene product or a chimeric CD47/CD200 gene and/or gene product.

In embodiments, the modified cell (or differentiated cell therefrom) does not express all three of CD47, CD24, and CD200. In embodiments, the modified cell that the BioNV is derived from is engineered such that BioNVs are stabilized, but not to a degree where the BioNVs are resistant to being cleared from the body. A BioNV that is too stable could eventually trigger a humoral response, resulting in limiting the number of doses or treatments that can be administered.

In embodiments, the modified cell expresses or has increased expression of a chimeric CTLA-4 gene and/or gene product. In embodiments, the modified cell expresses or has increased expression of a chimeric MFG-E8 gene and/or gene product. In embodiments, the modified cell expresses or has increased expression of an NCAM gene and/or gene product. In embodiments, the modified cell expresses or has increased expression of a chimeric α-phagocytic integrin gene and/or gene product.

In embodiments, the modified cell has reduced or ablated expression and/or activity of one or more immunogenic proteins, such as proteins that result in an immune response in the subject, donor-recipient mismatch, HLA alloimmunity, inflammation, CRS, and the like, such as MHC class I proteins, MHC class II proteins, HLA proteins, TCR proteins, CRS proteins, etc. In embodiments, the modified cell has reduced or ablated expression and/or activity of 3 or more immunogenic proteins, 4 or more immunogenic proteins, 5 or more immunogenic proteins, 6 or more immunogenic proteins, 7 or more immunogenic proteins, 8 or more immunogenic proteins, 9 or more immunogenic proteins, 10 or more immunogenic proteins, 11 or more immunogenic proteins, or 12 or more immunogenic proteins.

In embodiments, the modified cell has expression or increased expression and/or activity of one or more immunoprotective proteins, such as proteins that result prevent or reduce an immune response in the subject, prevent or reduce premature clearance of the BioNV in the subject, prevent or reduce phagocytosis, confer barrier-crossing functionality, and the like, such as CD47, CD24, CD200, CD34, CCL2, H2-M3, MFG-E8, PD-L1 (in non-activated cell sources), CTLA-4, etc. In embodiments, the modified cell expresses or has increased expression of 3 or more immunoprotective proteins, 4 or more immunoprotective proteins, 5 or more immunoprotective proteins, 6 or more immunoprotective proteins, 7 or more immunoprotective proteins, 8 or more immunoprotective proteins, 9 or more immunoprotective proteins, or 10 or more immunoprotective proteins.

In embodiments, “increased expression,” or “increased expression and/or activity,” as used herein, refers to an increase in expression and/or activity in the BioNV, and/or hypoimmunogenic cell derived therefrom, in comparison to a native, or wild-type cognate cell. For example, in embodiments, the increased expression and/or activity of one or more biomolecules described herein can confer the hypoimmunogenic properties of an iPSC relative to an iPSC which does not have the same expression pattern or expression level of the protein. In embodiments, the “increased expression and/or activity,” is due to a genetic amendment, such as a knock-in.

In embodiments, the modified cell is allogeneic. In embodiments, the modified cell does not cause an immune reaction in patients to which it or a BioNV derived therefrom is administered.

In embodiments, the BioNV comprises one or more targeting agents directed to the marker on its surface. In embodiments, BioNV used in methods herein comprise one or more chimeric antigen receptors (CARs) as the VERRs or viral ligands comprising the targeting agent. In embodiments, the CAR, VERR, and/or viral ligand lacks an intracellular domain. In embodiments, the CAR, VERR, and/or viral ligand comprises a targeting agent, a transmembrane domain, and an intracellular domain, which comprises a costimulatory domain and/or a signaling domain. In embodiments, the CAR, VERR, and/or viral ligand comprises a binding moiety oriented on the surface of the BioNV to allow binding to the marker. In embodiments, the targeting agent (or binding moiety thereof) is an antibody or antibody format. In embodiments, the antibody or antibody format is selected from one or more of a monoclonal antibody, polyclonal antibody, antibody fragment, VERR, viral ligand, Fab, Fab′, Fab′-SH, F(ab′)2, Fv, single chain Fv (scFv), diabody, nanobody, linear antibody, bispecific antibody, multi-specific antibody, chimeric antibody, humanized antibody, human antibody, and fusion protein comprising the antigen-binding portion of an antibody. In embodiments, the antibody format is a VERR-scFv fusion. In embodiments, the transmembrane domain is derived from CD28, CD37, CD4, CD8a, ICOS, or a fragment thereof. In embodiments, the intracellular domain comprises an intracellular signaling domain of a CD37-chain and/or one or more co-stimulatory molecules, optionally selected from CD28, 4-1BB, ICOS, CD27, OX40, or a fragment thereof.

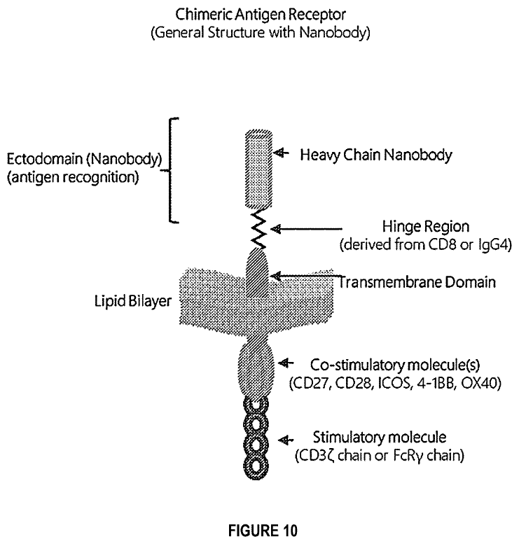

In embodiments, methods of treating, preventing, and/or ameliorating a viral infection include administering BioNVs with a VERR ligand-binding ectodomain or viral ligand (cell receptor ligand), CD8 or IgG4-derived hinge, transmembrane domain, co-stimulatory molecule(s) (e.g., CD27, CD28, ICOS, 4-1BB, and/or OX40), and/or stimulatory molecule (e.g., CD37 chain or FcRγ chain) (see, for example, FIG. 10).

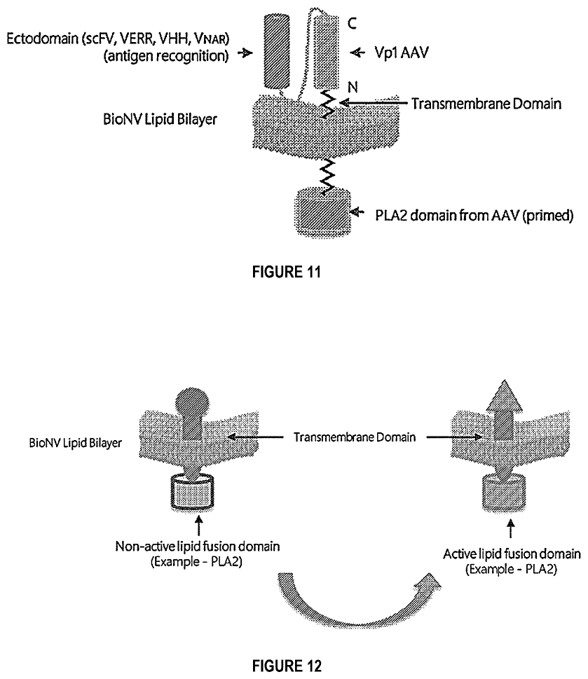

In embodiments, methods of treating, preventing, and/or ameliorating a viral infection include administering BioNVs with a CAR, scFV, VERR, VHH, VAR antigen-binding, and/or viral ligand fused with Vp1 AAV protein ectodomain, transmembrane domain, and PLA2 domain from AAV (primed). The PLA2 domain can be present on the external, solvent-exposed surface of the BioNV in a non-activated form. Alternatively, or additionally, the PLA2 domain can be engineered to become active from the inside of the BioNV (see, for example, FIG. 11).

In embodiments, methods of treating, preventing, and/or ameliorating a viral infection include administering BioNVs with surface-exposed transmembrane anchored membrane fusion protein(s) fused to internally oriented non-active (or activated) lipid fusion domains (e.g., PLA2) (see, for example, FIG. 12).

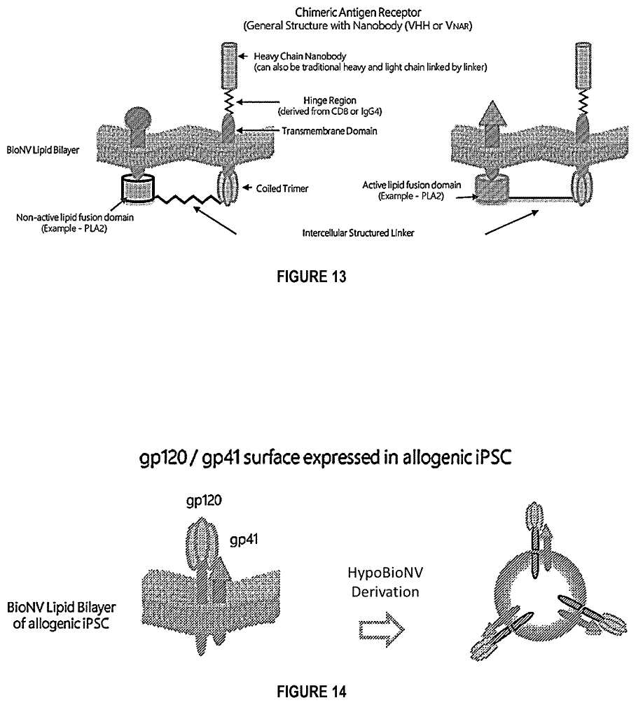

In embodiments, methods of treating, preventing, and/or ameliorating a viral infection include administering BioNVs with externally-oriented transmembrane anchored membrane lipid fusion protein and/or lipid protein complexes. The BioNVs are capable of fusion with the plasma membrane of target cells, resulting in the direct injection or deposit of the payload into the cytoplasm of the cell. The CAR (e.g., VHH, VERR, VNAR nanobody, or viral ligand) can have a generic internal coiled trimer which is linked to a PLA2 protein of the lipid protein complex (see, for example, FIG. 13, left). Upon binding of the CAR and/or VERR and/or viral ligand to the target, the coiled trimer can undergo a conformational change which activates the fusion protein (or protein complex) and initiates the internalization mechanism(s) of the target cell (see, for example, FIG. 13, right).

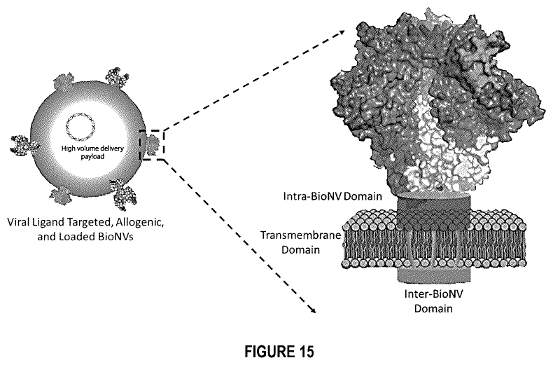

In embodiments, the methods of treating, preventing, and/or ameliorating a viral infection include administering BioNVs with a bispecific CAR. surface expressed HIV gp120/gp41 (e.g., as shown in FIG. 14). BioNVs expressing gp120/gp41 complexes can also facilitate fusion with the plasma membrane of a target cell. WT gp120/gp41 complexes on BioNVs can target CD4 receptors and/or CXCR4 and/or CCR5 co-receptors on latently HIV-infected lymphocytes to deliver therapeutic payloads, for example CRISPR/Cas-based gene editing machinery into the cytoplasm. Without wishing to be bound by theory, this delivery mechanism has the advantage of evading the canonical endosomal processing pathways. Surface epitopes of the gp120 receptor ligand can be mutated to target cellular markers other than CD4 receptors and/or CXCR4 and/or CCR5 co-receptors, increasing the treatment repertoire (see, for example, FIG. 15).

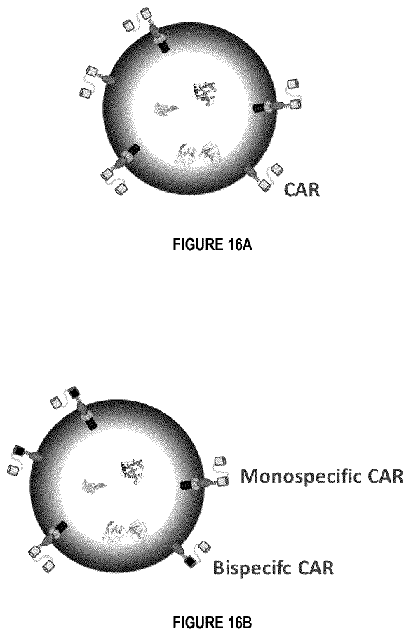

In embodiments, the BioNV comprises a bispecific CAR, for example as shown in FIGS. 16A-16B includes a gp120/gp41 complex to deliver the gene editing payload. In embodiments, the methods of treatment include administering a complex can be expressed on the surface of the cell (transmembrane) and be surface-exposed in the resulting BioNV comprising a bispecific CAR that targets two biomarkers in addition to administering one or more checkpoint inhibitors, and/or one or more antiretroviral therapies. (post-processing). In embodiments, the BioNV comprises a bispecific CAR that targets a checkpoint inhibitor biomarker (a target of a checkpoint inhibitor, e.g., PD-L1 or PD-L2, etc.) and a second biomarker (FIG. 16A). gp120/gp41 complex recognizes CD4 receptors and/or CXCR4 and/or CCR5 co-receptors on target cells and can be used to deliver a gene editor to cells that expresses these receptors. In embodiments, the BioNV comprises a bispecific CAR that targets a checkpoint inhibitor biomarker gp120/gp41 receptor complex is used to treat immune cells with viral infection because the complex combines a mechanism to recognize the target cells (via gp120 interacting with CD4 receptors) and a cell biomarker present on an HIV-infected cell (Siglec-1, CD34, etc.). mechanism for injecting the gene editor into the cytoplasm, thereby avoiding the less efficient endosomal pathway, via the gp41 interacting with the CXCR4 and/or CCR5 receptors. In embodiments, the BioNV can comprise a monospecific CAR and a bispecific CAR (FIG. 16B) where gp120 is the targeting portion of the complex and gp41 is the harpooning/fusogen portion of the complex. In embodiments, the gp120/gp41 complex is highly precise and there is reduced off-target delivery to unintentional cells.

In embodiments, the gp120/gp41 complex BioNVs are administered at higher doses for latently infected cells. In embodiments, latently infected cells are targeted with Siglec, PD-1, and/or CD32a (or a combination thereof) as transmembrane proteins in a construct in the BioNV to target a broader cell population.

In embodiments, the fusion of BioNVs to a cell can be mediated by the proteins as shown in one or more of FIG. 10, FIG. 11, FIG. 12, and/or FIG. 14.

BioNVs are, in embodiments, about 10-1200 nm in diameter. In embodiments, BioNVs are about 10 nm in size, about 20 nm in size, about 30 nm in size, about 40 nm in size, about 50 nm in size, about 60 nm in size, about 70 nm in size, about 80 nm in size, about 90 nm in size, about 100 nm in size, about 120 nm in size, about 140 nm in size, about 160 nm in size, about 180 nm in size, about 200 nm in size, about 300 nm in size, about 400 nm in size, about 500 nm in size, about 600 nm in size, about 700 nm in size, about 800 nm in size, about 900 nm in size, about 1000 nm in size, about 1100 nm in size, or about 1200 nm in size. In embodiments, BioNVs range in size from about 10 nm to 20 nm in size, about 20 nm to 30 nm in size, about 30 nm to 40 nm in size, about 40 nm to 50 nm in size, about 50 nm to 60 nm in size, about 60 nm to 70 nm in size, about 70 nm to 80 nm in size, about 80 nm to 90 nm in size, about 90 nm to 100 nm in size, about 10 nm to 100 nm in size, about 100 nm to 200 nm in size, about 200 nm to 400 nm in size, about 400 nm to 600 nm in size, about 600 nm to 800 nm in size, about 800 nm to 1000 nm in size, about 1000 nm to 1200 nm in size, or about 10 nm to 1200 nm in size.

In embodiments, the BioNV substantially lacks expression and/or activity of HLA-A, HLA-B, HLA-C, HLA-F, CIITA, IL-6, TRAC, TRBC, and one of either HLA-E or HLA-G.

In embodiments, the BioNV substantially lacks expression and/or activity of HLA-A, HLA-B, HLA-C, HLA-F, CIITA, IL-6, TRAC, TRBC, SerpinB9, and one of either HLA-E or HLA-G.

In embodiments, the BioNV substantially lacks expression and/or activity of HLA-A, HLA-B, HLA-C, HLA-F, CIITA, IL-6, TRAC, TRBC, SerpinB9, CD200, one of either HLA-E or HLA-G, and one or more of IL-4, IL-10, and IL-16.

In embodiments, the BioNV comprises a membrane-embedded α-phagocytic integrin, CCL2, H2-M3, FasL, MFG-E8, anti-IL-6R antibody or antibody format, and PD-L1 (in BioNVs derived from non-activated cell sources) and/or CTLA-4, and one of either CD24, CD47, CD200, chimeric CD24/CD47, chimeric CD24/CD200, and chimeric CD47/CD200, or two of either CD24, CD47, and CD200.

In embodiments, the BioNV comprises a membrane-embedded α-phagocytic integrin, CCL2, H2-M3, FasL, MFG-E8, anti-IL-6R antibody or antibody format, SerpinB9, and PD-L1 L1 (in BioNVs derived from non-activated cell sources) and/or CTLA-4, and one of either CD24, CD47, CD200, chimeric CD24/CD47, chimeric CD24/CD200, and chimeric CD47/CD200, or two of either CD24, CD47, and CD200.

In embodiments, the BioNV comprises a membrane-embedded CD200 protein and substantially lacks either CD24 or CD47. In embodiments, the BioNV substantially lacks protein and/or activity of SerpinB9 and CD200.

In embodiments, the checkpoint inhibiting agent is an agent that modulates one or more of PD-1, PD-L1, PD-L2, CTLA-4, TIM-3, LAG-3, B7-H3, B7-H4, A2aR, CD73, NKG2A, PVRIG/PVRL2, CEACAM1, CEACAM 5/6, FAK, CCL2/CCR2, LIF, CD47/SIRPα, CSF-1 (M-CSF)/CSF-1R, IL-1, IL-1R3, IL-8, SEMA4D, Ang-2, CLEVER-1, Axl, Phosphatidylserine. In embodiments, In embodiments, the checkpoint inhibiting agent is an agent that modulates one or more of PD-1 (e.g., Cemiplimab, Nivolumab, Pembrolizumab), PD-L1 (e.g., Atezolizumab, Avelumab, Durvalumab), PD-L2, CTLA-4 (e.g., Ipilimumab), TIM-3 (e.g., MBG453, Sym023, TSR-022), LAG-3 (e.g., LAG525 (IMP701), REGN3767 (R3767), BI 754,091, tebotelimab (MGD013), eftilagimod alpha (IMP321), FS118), B7-H3/B7-H4 (e.g., MGC018, FPA150), A2aR (e.g., EOS100850, AB928), CD73 (e.g., CPI-006), NKG2A (e.g., Monalizumab), PVRIG/PVRL2 (e.g., COM701), CEACAM1 (e.g., CM24), CEACAM 5/6 (e.g., NEO-201), FAK (e.g., Defactinib), CCL2/CCR2 (e.g., PF-04136309), LIF (e.g., MSC-1), CD47/SIRPα (e.g., Hu5F9-G4 (5F9), ALX148, TTI-662, RRx-001), CSF-1 (M-CSF)/CSF-1R (e.g., Lacnotuzumab (MCS110), LY3022855, SNDX-6352, emactuzumab (RG7155), pexidartinib (PLX3397)), IL-1/IL-1R3 (e.g., CAN04, Canakinumab (ACZ885)), IL-8 (e.g., BMS-986253), SEMA4D (e.g., Pepinemab (VX15/2503)), Ang-2 (e.g., Trebananib), CLEVER-1 (e.g., FP-1305), Axl (e.g., Enapotamab vedotin (EnaV)), and/or Phosphatidylserine (e.g., Bavituximab) (Marin-Acevedo, J. A., et al. “Next generation of immune checkpoint inhibitors and beyond.” J Hematol Oncol. Vol. 14, No. 45 (2021). https://doi.org/10.1186/s13045-021-01056-8). In embodiments, the checkpoint inhibiting agent used in methods described herein is an antibody or antibody format.

In embodiments, the antibody or antibody format is selected from one or more of a monoclonal antibody, polyclonal antibody, antibody fragment, Fab, Fab′, Fab′-SH, F(ab′)2, Fv, single chain Fv, diabody, nanobody, linear antibody, bispecific antibody, multi-specific antibody, chimeric antibody, humanized antibody, human antibody, and fusion protein comprising the antigen-binding portion of an antibody. In embodiments, the antibody or antibody format specific for PD-1 is selected from nivolumab, pembrolizumab, and pidilizumab. In embodiments, the antibody or antibody format specific for PD-L1 is selected from atezolizumab, avelumab, durvalumab, and BMS-936559. In embodiments, the antibody or antibody format specific for CTLA-4 is selected from ipilimumab, tremelimumab, AGEN1884, and RG2077.

In embodiments, the viral infection is a latent infection. In embodiments, the viral infection is a retroviral infection. In embodiments, the retroviral infection is a human immunodeficiency virus (HIV) infection. The HIV can be HIV-1 or HIV-2. HIV-1 can refer to one of Group M, Group N, Group O, Group P, or any combination thereof. In embodiments, the HIV-1 is Group M and a subtype selected from subtype A, B, C, D, F, G, H, I, J, K, and L. In embodiments, the retroviral infection is one of bovine immunodeficiency virus, caprine arthritis encephalitis virus, equine infectious anemia virus, feline immunodeficiency virus, Jembrana disease virus, Puma lentivirus, simian immunodeficiency virus, visna-maedi virus, and human T-lymphotropic virus type 1 (HTLV-1) and HTLV-2.

In embodiments, the BioNV encapsulates a payload; e.g., “lumen-loading”, or the ability of the BioNV to have a payload loaded into the lumen (space in the biomimetic nanovesicle). In embodiments, the payload is one or more of a biologic, a nucleic acid, a fusion protein, a fluorescent protein, a tracing dye, a radionuclide, and/or a small molecule. In embodiments, the payload is a therapeutic payload for a virus type that the CAR and/or VERR and/or viral ligand is targeted against.

In embodiments, the BioNV encapsulates one or more perforin molecules. In embodiments, the BioNV comprises one or more granzyme molecules. In embodiments, the granzyme molecules are selected from granzyme A, B, H, K, and M. In embodiments, the BioNV encapsulates one or more perforin and/or one or more granzyme molecules derived from a cell from which the BioNV is derived. In embodiments, the BioNV encapsulates one or more perforin and/or one or more granzyme molecules exogenously added to the BioNV.

In embodiments, the methods described herein can make use of BioNVs to deliver a therapeutic payload to virally infected cells. In embodiments, the BioNV can be used to deliver a gene editor. In embodiments, the BioNV can be used for gene delivery, where BioNVs are derived from cells with or without activation. In embodiments, BioNVs can be used to deliver a gene editor to excise HIV elements from the host genome by targeting Siglec-containing cells, CD32a, CD4, or any other target or combinations of targets described herein.

In embodiments, the payload includes gene editing nucleic acids and/or proteins, such as for example, TALENs, ZFNs, RNase P RNA, C2c1, C2c2, C2c3, Cas9, Cpf1, TevCas9, Archaea Cas9, CasY.1, CasY.2, CasY.3, CasY.4, CasY.5, CasY.6, CasX Cas omega, transposase, and/or any ortholog or homolog of any of these editors. In embodiments, the gene editors can also include gRNA, which, as used herein, refers to guide RNA. In embodiments, the gRNA can be a sequence complimentary to a coding or a non-coding sequence and can be tailored to the particular sequence to be targeted. In embodiments, the gRNA can be a sequence complimentary to a protein coding sequence, for example, a sequence encoding one or more viral structural proteins, (e.g., gag, pol, env and tat). In embodiments, the gRNA sequence can be a sense or anti-sense sequence. In embodiments, when a gene editor composition is administered herein, preferably without limitation, including two or more gRNAs; however, a single gRNA can also be used.

In embodiments, the BioNV encapsulates both a gene editing payload and an antiretroviral therapy. In embodiments, the BioNV can encapsulate one or more ART drugs and/or one or more gene editing payloads. In embodiments, the virus can be excised from the cell by the gene editing payload and the ART drug can be simultaneously delivered to prevent reactivation of the virus.

In embodiments, the BioNV encapsulates a gene editing payload. In embodiments, the gene editing payload comprises one or more gene editor nucleic acids and/or proteins, or one or more nucleic acids encoding one or more gene editors. In embodiments, the one or more gene editors is a site-directed endonuclease, TALEN, ZFN, RNase P RNA, CRISPR/Cas nuclease, C2c1, C2c2, C2c3, Cas9, Cpf1, TevCas9, Archaea Cas9, CasY.1, CasY.2, CasY.3, CasY.4, CasY.5, CasY.6, CasX, Cas omega, transposase, and/or any ortholog or homolog thereof. In embodiments, the gene editing payload comprises a transactivating response region (TAR) loop system.

In embodiments, the gene editing payload is delivered to virally infected cells for ‘cutting out’ or otherwise excising integrated virus. For example, in embodiments, the BioNV can deliver gene editing guides for HIV to 1) cut out the entire 9,000+ bp genome and/or, 2) cut out segment(s) of the virus that are within 500 bp of one another, such as scrambling the viral genome with multiple guides, and/or 3) deliver guides that target regulatory regions of the viral genome such as Ras Binding Elements (RBEs I through IV). In embodiments, the guides for targeting RBEs are designed so that part of the guide binds outside the RBE to prevent endogenous off-targeting events. Without wishing to be bound by theory, if the guides are designed to bind only within the RBE, then they may bind to other non-viral sites within the human genome. In embodiments, to prevent such off-targeting, the overhangs that bind to the outside of the RBEs are thus designed to be highly specific to the virus sequences only. In embodiments, HIV LTRs that are targeted. In embodiments, the gene editing payloads target one, two, three, or all four elements spread across each LTR.

In embodiments, the methods comprise administering one or more antiretroviral therapies. In embodiments, the BioNV includes the one or more antiretroviral therapies. In embodiments, the antiretroviral therapy includes a non-nucleoside reverse transcriptase inhibitor (NNRTI), optionally selected from efavirenz (SUSTIVA), rilpivirine (EDURANT) and doravirine (PIFELTRO). In embodiments, the antiretroviral therapy includes a nucleoside or nucleotide reverse transcriptase inhibitor (NRTI), optionally selected from abacavir (ZIAGEN), tenofovir (VIREAD), emtricitabine (EMTRIVA), lamivudine (EPIVIR), zidovudine (RETROVIR), emtricitabine/tenofovir (TRUVADA), and emtricitabine/tenofovir alafenamide (DESCOVY). In embodiments, the antiretroviral therapy includes a protease inhibitor (PI), optionally selected from atazanavir (REYATAZ), darunavir (PREZISTA), and lopinavir/ritonavir (KALETRA). In embodiments, the antiretroviral therapy includes an integrase inhibitor, optionally selected from bictegravir sodium/emtricitabine/tenofovir alafenamide fumar (BIKTARVY), raltegravir (ISENTRESS), and dolutegravir (TIVICAY). In embodiments, the antiretroviral therapy includes an entry or fusion inhibitor, optionally selected from enfuvirtide (FUZEON) and maraviroc (SELZENTRY).

In embodiments, the antiretroviral therapy includes at least two, or at least three, or at least four of the antiretroviral therapies in combination. In embodiments, the BioNV encapsulates one or more antiretroviral therapies.

In embodiments, methods of treatment herein include co-administering a whole cell therapy (e.g., T cell, NK cell, TIL, macrophage therapy). In embodiments, supplementing a whole cell therapy with BioNVs can be used to decrease the effective dosage of the whole cell therapy needed, reducing CRS, off-target effects, and the like.

In embodiments, methods of treatment herein include administering an additional therapeutic agent. In embodiments, the additional therapeutic agent can be any additional anti-cancer agent, analgesic, and/or non-steroidal inflammatory agent (NSAID).

In embodiments, BioNVs can be frozen at −80 °C and/or lyophilized (e.g., for reconstitution in buffer). In embodiments, BioNVs can be stable at about ambient temperature, at about −20 °C, at about 4 °C, at about 25 °C, or at about 37 °C for at least about 1 hour, at least about 2 hours, at least about 4 hours, at least about 6 hours, at least about 12 hours, at least about 24 hours, at least about 2 days, at least about one week, or at least about one month or longer.

In embodiments, the methods described herein cause the excision of viral nucleic acids from one or more infected cells, optionally HIV-infected cells. In embodiments, the methods described herein cause the excision of viral nucleic acids from one or more latently infected cells, optionally latently retroviral-infected cells, such as HIV-infected cells. In embodiments, the methods described herein cause a prevention of clonal expansion of infected cells, optionally HIV-infected cells. In embodiments, methods described herein cause eradication, stoppage, halt or end of HIV or infection symptoms, or the progression of the symptoms or virus in the subject. In embodiments, methods described herein induce and/or maintain sustained viral control, optionally selected from undetectable levels of plasma viremia and/or maintenance of less than about 1000 copies/mL of HIV viral RNA or cDNA, less than about 500 copies/mL of HIV viral RNA or cDNA, less than about 100 copies/mL of HIV viral RNA or cDNA, less than about 50 copies/mL of HIV viral RNA or cDNA, or less than about 10 copies/mL of HIV viral RNA or cDNA, optionally as assayed by a polymerase chain reaction (PCR) test, a branched chain DNA (bDNA) test or a nucleic acid sequence-based amplification (NASBA) test.

### Dosing and Administration

The dosage of any BioNVs disclosed herein as well as the dosing schedule can depend on various parameters and factors, including, but not limited to, the specific BioNVs, the disease being treated, the severity of the condition, whether the condition is to be treated or prevented, the subject's age, weight, and general health, and the administering physician's discretion. Additionally, pharmacogenomic (the effect of genotype on the pharmacokinetic, pharmacodynamic or efficacy profile of a therapeutic) information about a particular subject may affect dosage used. Furthermore, the exact individual dosages can be adjusted somewhat depending on a variety of factors, including the specific combination of the agents being administered, the time of administration, the route of administration, the nature of the formulation, the rate of excretion, the disease being treated, the severity of the disorder, and the anatomical location of the disorder. Some variations in the dosage can be expected.

In embodiments, delivery of BioNVs can be like that of a vesicle, in particular a liposome (see Langer, 1990, Science 249:1527-1533; Treat et al., in Liposomes in Therapy of Infectious Disease and Cancer, Lopez-Berestein and Fidler (eds.), Liss, New York, pp. 353-365 (1989)).

Methods of treatment using BioNVs described herein, in embodiments, include dosage ranges in concentration of number of BioNVs per kilogram (kg) subject body weight. In embodiments, suitable dosage ranges for methods of treatment described herein can include from about 10³ BioNVs/kg to about 10¹² BioNVs/kg. In embodiments, the BioNVs are present in the composition at a concentration of about 10³ BioNVs/mL to about 10¹⁴ BioNVs/mL. Alternatively, in embodiments, BioNV compositions are present in compositions as weight/volume in the range of about 5 ng/ml to about 500 mg/mL. In embodiments, the BioNV dosages are based on the size of the BioNVs used for the treatment, for example, BioNVs at 1000 nm are provided in approximately 5-fold to 10-fold fewer amounts than 100 nm BioNVs for a comparable dose.

In embodiments, BioNVs disclosed herein are administered by a controlled-release or a sustained-release means or by delivery of a device that is well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled or sustained-release of one or more active ingredients using, for example, hydropropylmethyl cellulose, other polymer matrices, gels, permeable membranes, osmotic systems, multilayer coatings, microparticles, microspheres, or a combination thereof, to provide the desired release profile in varying proportions. Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In embodiments, polymeric materials are used (see Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; Levy et al., 1985, Science 228:190; During et al., 1989, Ann. Neurol. 25:351; Howard et al., 1989, J. Neurosurg. 71:105).

In embodiments, a controlled-release system is placed in proximity of the target area to be treated, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533 may be used.

In embodiments, the methods using BioNVs include applying BioNVs to a surface of a device (e.g., a catheter) or contained within a pump, patch, or other drug delivery device. The excipient or carrier can be selected based on the mode and route of administration. Suitable pharmaceutical carriers, as well as pharmaceutical necessities for use in pharmaceutical formulations, are described in Remington's Pharmaceutical Sciences (E. W. Martin), a well-known reference text in this field, and in the USP/NF (United States Pharmacopeia and the National Formulary).

Pembrolizumab is a humanized anti-PD-1 monoclonal antibody that is used in treating cancers such as melanoma, lung cancer, head and neck cancer, Hodgkin lymphoma, stomach cancer, cervical cancer, and breast cancer. It binds PD-1 on the cancer cell, blocking the interaction with the receptors of the lymphocytes. In embodiments, this allows the immune system to target the cancer cells which can no longer evade the cytotoxic response, while preventing the immune system from attacking healthy cells. In embodiments, pembrolizumab can be administered at doses of 200 mg to 400 mg or 2 mg/kg.

Nivolumab is a humanized anti-PD-1 monoclonal antibody that is used in treating cancers such as melanoma, lung cancer, malignant pleural mesothelioma, renal cell carcinoma, colon cancer, esophageal squamous cell carcinoma, liver cancer, gastric cancer, and esophageal or gastroesophageal junction cancer. In embodiments, nivolumab works in the same manner as pembrolizumab. In embodiments, nivolumab can be administered at doses of 240 mg to 480 mg or 3 mg/kg.

In embodiments, BioNVs can be administered at dosages that are congruent to dosages of pembrolizumab and/or nivolumab. In embodiments, BioNVs can be administered at doses that are congruent to dosages of whole cells, for example, based on CAR concentration. In embodiments, the typical concentration range of CAR protein per microgram of T cells is between 0.20 ng-0.70 ng, whereas a single BioNV may have a total number of CARs that is 5 times to 10,000 times less than the whole cell, resulting in a conversion of BioNV mass to CAR concentration, where the CAR concentration can be assumed equivalent (such as the case in exosomes) or increased (such as the case in BioNVs) to the cell from which it originated (e.g., the T cell). In embodiments, the concentration and/or surface density of the targeting agent (e.g., CAR) is increased on the BioNV compared to the whole cell from which is it derived. In embodiments, the concentration and/or surface density of the targeting agent (e.g., CAR) is enriched by serial extrusion processing of the whole cell. In embodiments, the concentration and/or surface density of the targeting agent (e.g., CAR), among other cell surface molecules, on the BioNV is 2-fold to 100-fold increased relative to the whole cell. In embodiments, due to exosomes being naturally shed, their concentration and/or surface density of the targeting agent (e.g., CAR), among other cell surface molecules, is substantially the same as the whole cell.

The dosage regimen utilizing any BioNVs disclosed herein can be selected in accordance with a variety of factors including type, species, age, weight, sex, and medical condition of the subject; the severity of the condition to be treated; the route of administration; the renal or hepatic function of the subject; the pharmacogenomic makeup of the individual; and the specific composition of the invention employed. Any BioNVs disclosed herein can be administered in a single daily dose, or the total daily dosage can be administered in divided doses of two, three, or four times daily. Furthermore, any BioNVs disclosed herein can be administered continuously rather than intermittently throughout the dosage regimen.

In embodiments, BioNVs are administered in consecutive doses about every hour, about every 2 hours, about every 6 hours, about every 12 hours, about every 24 hours, about every 2 days, about every 4 days, about every 7 days, about every 2 weeks, about every 4 weeks, or about every month.

In embodiments, a combined remission or clinical remission of the disease or disorder, or non-detection of viral DNA or RNA is achieved within about 24 weeks, about 18 weeks, about 12 weeks, about 8 weeks, about 6 weeks, about 4 weeks, about 2 weeks, or about 1 week from administration of the composition and methods with such compositions.

### Compositions

In aspects, the present disclosure relates to compositions for the treatment of viral infection comprising an allogeneic, BioNV comprising one or more membrane-embedded viral epitope recognition receptor (VERR) and/or a viral ligand or marker of cellular infection (e.g., Siglec-1, CD4, CXCR4, CCR5, PD-1, CD32a), one or more membrane-embedded proteins of an α-phagocytic integrin, CCL2, H2-M3, FasL, MFG-E8, PD-L1 (in BioNVs derived from non-activated cell sources) and/or CTLA-4, and SerpinB9, a membrane-embedded protein of one of either CD24, CD47, CD200, chimeric CD24/CD47, chimeric CD24/CD200, and chimeric CD47/CD200, or two of either CD24, CD47, and CD200; and a membrane substantially lacking proteins of one or more of HLA-A, HLA-B, HLA-C, HLA-F, CIITA, IL-6, TRAC, TRBC, one of either HLA-E or HLA-G, and one or more of IL-4, IL-10, and/or IL-16, and a checkpoint inhibitor.

In aspects, the present disclosure relates to compositions for the treatment of viral infection comprising an allogeneic, hypoimmunogenic biomimetic nanovesicle (BioNV) comprising one or more membrane-embedded targeting agents targeted against one or more markers of viral infection, a viral epitope recognition receptor (VERR), and/or a viral ligand a marker of cellular infection (e.g., Siglec-1, CD4, CXCR4, CCR5, PD-1, CD32a), one or more membrane-embedded proteins of an α-phagocytic integrin, CCL2, H2-M3, FasL, MFG-E8, PD-L1 (in BioNVs derived from non-activated cell sources) and/or CTLA-4, a membrane-embedded protein of one of either CD24, CD47, CD200, chimeric CD24/CD47, chimeric CD24/CD200, and chimeric CD47/CD200, or two of either CD24, CD47, and CD200; and a membrane substantially lacking proteins of one or more of HLA-A, HLA-B, HLA-C, HLA-F, CIITA, IL-6, TRAC, TRBC, SerpinB9, CD200, one of either HLA-E or HLA-G, and one or more of IL-4, IL-10, and/or IL-16, and a checkpoint inhibitor.

In embodiments, compositions include BioNVs. In embodiments, compositions include a BioNV and at least one checkpoint inhibitor, as described herein. In embodiments, the BioNVs can adsorb therapeutic molecules onto the surface of the NV and/or encapsulate a therapeutic payload within an aqueous compartment of the NV. In embodiments, compositions include a BioNV and at least one checkpoint inhibitor encapsulated within the aqueous core. In embodiments, the composition comprises a therapeutically effective amount of the BioNVs and/or a therapeutically effective amount of at least one checkpoint inhibitor. In embodiments, the BioNV and checkpoint inhibitor(s) can be combined in solution or can be in separate solutions to be co-administered.

In embodiments, the composition is allogeneic and/or hypoimmunogenic. In embodiments, the composition is derived from iPSCs (among other cell types) which have been modified to reduce expression of immunogenic molecules and/or increase expression of immunoprotective molecules.

In embodiments, the composition is hypoimmunogenic. For example, in embodiments, the composition does not result in an inflammatory reaction and/or an immune response upon administration. In embodiments, the BioNVs are hypoimmunogenic. In embodiments, upon administration to a subject, the composition, optionally the BioNVs therein, elicits less than about 50%, about 45%, about 40%, about 35%, about 30%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8, about 7%, about 6%, about 5%, about 4%, about 3%, about 2%, or about 1% of an inflammatory or immune response measured as a function of cytokine, chemokine, or immunomodulatory enzyme concentration, such as IL-1, IL-2, IL-3, IL-4, IL-6, IL-7, IL-9, IL-10, IL-11, IL-12, IL-13, IL-14, IL-15, IL-16, IL-17, IL-20, IFN-α/β/γ, TNFα/β, IDO, HLA-G, HGF, PGE2, among others, or any combination thereof, in comparison, e.g. to a cognate whole cell therapy counterpart.

In embodiments, the BioNVs are present in the composition at a concentration of about 10³ BioNVs/mL to about 10¹⁴ BioNVs/mL. Alternatively, in embodiments, BioNV compositions are present in compositions as weight/volume in the range of about 5 ng/ml to about 500 mg/mL.

In embodiments, the composition is substantially free of one or more bacteria, virus, fungus, spore, mycoplasma, pyrogen, and in more particular embodiments, is substantially free of all the foregoing. In embodiments, the composition is substantially free of whole cells and intracellular cell components including organelles such as nuclei, mitochondria, Golgi, etc., and/or substantially free of non-CAR and/or VERR and/or viral ligand-expressing NVs and/or substantially free of ruptured, damaged NVs. In embodiments, the composition is substantially free of cellular chromatin, nucleosomes, and other genetic material and non-therapeutic nucleic acids. In embodiments, BioNVs and BioNV compositions are substantial free of cellular genomic DNA.

### Pharmaceutical Compositions and Formulations

In aspects, the composition is a pharmaceutical composition. In embodiments, the pharmaceutical compositions of the present invention are formulated to provide a therapeutically effective amount of BioNVs as the active ingredient. In embodiments, the pharmaceutical compositions of the present invention are formulated to provide a therapeutically effective amount of one or more checkpoint inhibitors as a payload within a BioNV as the active ingredient. Typically, the pharmaceutical compositions also comprise one or more pharmaceutically acceptable excipients, carriers, including inert solid diluents and fillers, diluents, including sterile aqueous solution and various organic solvents, permeation enhancers, solubilizers and adjuvants.

Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea, and the like. In addition, auxiliary, stabilizing, thickening, lubricating, and coloring agents can be used. Pharmaceutically acceptable excipients are generally sterile when administered to a subject. Water is a useful excipient when any agent disclosed herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. Any composition disclosed herein, if desired, can also formulated with wetting or emulsifying agents, or pH buffering agents. Other examples of suitable pharmaceutical excipients are described in Remington's Pharmaceutical Sciences 1447-1676 (Alfonso R. Gennaro eds., 19^{th} ed. 1995), incorporated herein by reference.

In embodiments, the composition comprises an excipient or carrier. In embodiments, the diluent is a pharmaceutically acceptable excipient or carrier.

In embodiments, the pharmaceutical composition comprises a pharmaceutically acceptable diluent. Non-limiting example of diluents include liquid diluents such as water, ethanol, propylene glycol, glycerin, and various combinations thereof, and inert solid diluents such as calcium carbonate, calcium phosphate or kaolin. In embodiments, the diluent comprises one or more of saline, phosphate buffered saline, Dulbecco's Modified Eagle Medium (DMEM), alpha modified Minimal Essential Medium (alpha MEM), Roswell Park Memorial Institute Media 1640 (RPMI Media 1640), HBSS, human albumin, Ringer's solution, and the like, or any combination thereof.

In embodiments, the active ingredient is typically mixed with an excipient, diluted by an excipient or enclosed within such a carrier in the form of, for example, a capsule, tablet, sachet, paper, or other container. When the excipient serves as a diluent, it can be a solid, semisolid, or liquid material (e.g., normal saline), which acts as a vehicle, carrier or medium for the active ingredient. In embodiments, the compositions can be in the form of tablets, pills, powders, lozenges, sachets, cachets, elixirs, suspensions, emulsions, solutions, syrups, aerosols (as a solid or in a liquid medium), lotions, creams, ointments, gels, soft and hard gelatin capsules, suppositories, sterile injectable solutions, and sterile packaged powders. As is known in the art, the type of diluent can vary depending upon the intended route of administration. In embodiments, the resulting compositions can include additional agents, such as preservatives, cryopreservatives (e.g., DMSO), and/or lyoprotectants (e.g., polyols, salts). In embodiments, the carrier can be, or can include a lipid-based or polymer-based colloid. In embodiments, the carrier material can be a colloid formulated as a liposome, a hydrogel, a microparticle, a nanoparticle, or a block copolymer micelle. In embodiments, the carrier material can form a capsule, and that material may be a polymer-based colloid.

In embodiments, the pharmaceutical compositions comprising the BioNVs include a solubilizing agent. In embodiments, the pharmaceutical compositions comprising the BioNVs include a cryoprotective agent or an agent to improve thermal stability, such as DMSO or glycerol. The pharmaceutical compositions, in embodiments, can be delivered with a suitable vehicle or delivery device as known in the art.

In embodiments, the composition comprises a scaffold. In embodiments, the scaffold comprises biomaterials. In a non-limiting example, the three-dimensional biomaterials include BioNVs embedded in an extracellular matrix attached to, or dispersed within, or trapped within the scaffold. In embodiments, the biomaterials are biodegradable and/or synthetic.

In embodiments, the scaffold comprises biodegradable biomaterials. Non-limiting examples of biodegradable biomaterials include fibrin, collagen, elastin, gelatin, vitronectin, fibronectin, laminin, reconstituted basement membrane matrix, starch, dextran, alginate, hyaluronan, chitin, chitosan, agarose, sugars, hyaluronic acid, poly(lactic acid), poly(glycolic acid), polyethylene glycol, decellularized tissue, self-assembling peptides, polypeptides, glycosaminoglycans, derivatives and mixtures thereof. Other useful biodegradable polymers or polymer species include, but are not limited to, polydioxanone, polycarbonate, polyoxalate, poly(α-ester), polyanhydride, polyacetate, polycaprolactone, poly(ortho Esters), polyamino acids, polyamides, and mixtures and copolymers thereof, L-lactic acid and D-lactic acid stereopolymers, copolymers of bis(para-carboxyphenoxy) propanoic acid and sebacic acid, sebacic acid copolymers, caprolactone Copolymer, poly(lactic acid)/poly(glycolic acid)/polyethylene glycol copolymer, polyurethane and poly(lactic acid) copolymer, polyurethane and poly(lactic acid) copolymer, α-amino acid copolymer, α-amino acid and caproic acid copolymer, A-benzylglutamate and polyethylene glycol copolymers, succinate and poly(glycol) copolymers, polyphosphazenes, polyhydroxy-alkanoates and mixtures thereof. Binary and ternary systems are also contemplated. In embodiments, the scaffold comprises one or more of collagen, various proteoglycans, alginate-based substrates, and chitosan. In embodiments, the scaffold comprises one or more of a hydrogel, silk, Matrigel, acellular and/or decellarized scaffolds, poly-ε-caprolactone scaffolds, resorbable scaffolds, and nanofiber-hydrogel composite.

In embodiments, the scaffold comprises synthetic biomaterials. Non-limiting examples of synthetic biomaterials include lactone-based polyesters or copolyesters such as polylactide, polycaprolactonglycolide, polyorthoesters, polyanhydrides, polyaminoacids, polysaccharides, polyphosphazenes, poly(ether-ester) copolymers (e.g., PEO-PLLA); polydimethylsiloxane, poly(ethylene-vinylacetate), acrylate-based polymers or copolymers (e.g., polyhydroxyethyl methylmethacrylate, polyvinyl pyrrolidinone), fluorinated polymers such as polytetrafluoroethylene and cellulose esters.

In embodiments, the compositions can be prepared in any manner well known in the pharmaceutical arts, and can be administered by a variety of routes (e.g., subcutaneous, intravenous, etc.) depending upon whether local or systemic treatment is desired and upon the area to be treated. In embodiments, administration can be topical (including ophthalmic and to mucous membranes including intranasal, vaginal, and rectal delivery), pulmonary (e.g., by inhalation or insufflation of powders or aerosols, including by nebulizer; intratracheal, intranasal, epidermal and transdermal), ocular, oral, or parenteral. In embodiments, methods can include ocular delivery, topical administration (eye drops), subconjunctival, periocular or intravitreal injection or introduction by balloon catheter or ophthalmic inserts surgically placed in the conjunctival sac. In embodiments, parenteral administration includes intravenous, intra-arterial, subcutaneous, intraperitoneal or intramuscular injection or infusion; or intracranial, e.g., intrathecal or intraventricular administration. In embodiments, parenteral administration can be in the form of a single bolus dose, or may be, for example, by a continuous perfusion pump.

In embodiments, pharmaceutical compositions and formulations for topical administration may include transdermal patches, ointments, lotions, creams, gels, drops, suppositories, sprays, liquids, powders, and the like. In embodiments, methods of treating and/or preventing cancer include the use of pharmaceutical carriers, aqueous, powder or oily bases, thickeners, and the like.

In embodiments, the pharmaceutical compositions contain, as the active ingredient, nucleic acids and vectors described herein in combination with one or more pharmaceutically acceptable carriers. In embodiments, the terms “pharmaceutically acceptable” (or “pharmacologically acceptable”) refer to molecular entities and compositions that do not produce an adverse, allergic or other untoward reaction, when administered to an animal or a human, as appropriate. The methods and compositions disclosed herein can be applied to a wide range of species, e.g., humans, non-human primates (e.g., monkeys), horses or other livestock, dogs, cats, ferrets or other mammals kept as pets, rats, mice, or other laboratory animals. In embodiments, the term “pharmaceutically acceptable carrier,” includes any and all solvents, dispersion media, coatings, antibacterial, isotonic and absorption delaying agents, buffers, excipients, binders, lubricants, gels, surfactants and the like, that may be used as media for a pharmaceutically acceptable substance.

In embodiments, the compositions can be applied to a surface of a device (e.g., a catheter) or contained within a pump, patch, or other drug delivery device. In embodiments, the compositions can be administered alone, or in a mixture, in the presence of a pharmaceutically acceptable excipient or carrier (e.g., physiological saline). The excipient or carrier is selected based on the mode and route of administration. Suitable pharmaceutical carriers, as well as pharmaceutical necessities for use in pharmaceutical formulations, are described in Remington's Pharmaceutical Sciences (E. W. Martin), a well-known reference text in this field, and in the USP/NF (United States Pharmacopeia and the National Formulary).

In embodiments, the pharmaceutical composition further comprises a therapeutically effective amount of one or more agents for targeting PD-1. In embodiments, the one or more agents for targeting PD-1 is an antibody or antibody format. In embodiments, the one or more agents for targeting PD-1 is selected from nivolumab, pembrolizumab, and pidilizumab.

In embodiments, the pharmaceutical composition further comprises a therapeutically effective amount of one or more agents for targeting PD-L1. In embodiments, the one or more agents for targeting PD-L1 is an antibody or antibody format. In embodiments, the one or more agents for targeting PD-L1 is selected from atezolizumab, avelumab, durvalumab, and BMS-936559.

In embodiments, the pharmaceutical composition further comprises a therapeutically effective amount of one or more agents for targeting CTLA-4. In embodiments, the one or more agents for targeting CTLA-4 is an antibody or antibody format. In embodiments, the one or more agents for targeting CTLA-4 is selected from ipilimumab, tremelimumab, AGEN1884, and RG2077.

In embodiments, the compositions, e.g., pharmaceutical compositions, disclosed herein are suspended in a saline buffer (including, without limitation, TBS, PBS, and the like).

The present technology includes the disclosed BioNVs in various formulations of pharmaceutical compositions. BioNVs disclosed herein, in embodiments, can take the form of solutions, suspensions, emulsion, drops, tablets, pills, pellets, capsules, capsules containing liquids, powders, sustained-release formulations, emulsions, aerosols, sprays, suspensions, or any other form suitable for use.

Pharmaceutical compositions comprising the BioNVs described herein may conveniently be presented in unit dosage forms and may be prepared by any of the methods well known in the art of pharmacy. Such methods generally include the step of bringing therapeutic agents into association with a carrier, which constitutes one or more accessory ingredients. Typically, the pharmaceutical compositions are prepared by uniformly and intimately bringing therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In embodiments, any BioNVs disclosed herein are formulated in accordance with routine procedures as a pharmaceutical composition adapted for a mode of administration disclosed herein.

### Additional Therapeutic Agents

In embodiments, the compositions or methods described herein further comprise a therapeutically effective amount of one or more additional therapeutic agents. In embodiments, the therapeutically effective amount of one or more additional therapeutic agents may be in solution with a BioNV, adsorbed onto the surface of the NV, or a payload encapsulated within a BioNV. In embodiments, the additional therapeutic agent is one or more of a checkpoint inhibitor, an analgesic, and/or an anti-infective agent.

In embodiments, the additional therapeutic agent in the composition further comprises a therapeutically effective amount of one or more antiretroviral therapeutic agents. In embodiments, the antiretroviral therapy includes a non-nucleoside reverse transcriptase inhibitor (NNRTI), optionally selected from efavirenz (SUSTIVA), rilpivirine (EDURANT) and doravirine (PIFELTRO). In embodiments, the antiretroviral therapy includes a nucleoside or nucleotide reverse transcriptase inhibitor (NRTI), optionally selected from abacavir (ZIAGEN), tenofovir (VIREAD), emtricitabine (EMTRIVA), lamivudine (EPIVIR), zidovudine (RETROVIR), emtricitabine/tenofovir (TRUVADA), and emtricitabine/tenofovir alafenamide (DESCOVY). In embodiments, the antiretroviral therapy includes a protease inhibitor (PI), optionally selected from atazanavir (REYATAZ), darunavir (PREZISTA), and lopinavir/ritonavir (KALETRA). In embodiments, the antiretroviral therapy includes an integrase inhibitor, optionally selected from bictegravir sodium/emtricitabine/tenofovir alafenamide fumar (BIKTARVY), raltegravir (ISENTRESS), and dolutegravir (TIVICAY). In embodiments, the antiretroviral therapy includes an entry or fusion inhibitor, optionally selected from enfuvirtide (FUZEON) and maraviroc (SELZENTRY). In embodiments, the antiretroviral therapy comprises at least two, or at least three, or at least four of the antiretroviral therapies in combination. In embodiments, the BioNV encapsulate one or more different antiretroviral therapeutic agents. In embodiments, the BioNV composition includes one or more different ART agents outside of the NV.

In embodiments, the present compositions or methods contemplate other additional therapeutic agents, for example, an analgesic, to aid in treating inflammation or pain at the site of the administration, or an anti-infective agent to prevent infection of the site of treatment with the composition. Non-limiting examples of additional therapeutic agents include analgesics, such as nonsteroidal anti-inflammatory drugs, opiate agonists and salicylates; anti-infective agents, such as anthelmintics, antianaerobics, antibiotics, aminoglycoside antibiotics, antifungal antibiotics, cephalosporin antibiotics, macrolide antibiotics, miscellaneous B-lactam antibiotics, penicillin antibiotics, quinolone antibiotics, sulfonamide antibiotics, tetracycline antibiotics, antimycobacterials, antituberculosis antimycobacterials, antiprotozoals, antimalarial antiprotozoals, antiviral agents, anti-retroviral agents, scabicides, anti-inflammatory agents, corticosteroid anti-inflammatory agents, antipruritics/local anesthetics, topical anti-infectives, antifungal topical anti-infectives, antiviral topical anti-infectives; electrolytic and renal agents, such as acidifying agents, alkalinizing agents, diuretics, carbonic anhydrase inhibitor diuretics, loop diuretics, osmotic diuretics, potassium-sparing diuretics, thiazide diuretics, electrolyte replacements, and uricosuric agents; enzymes, such as pancreatic enzymes and thrombolytic enzymes; gastrointestinal agents, such as antidiarrheals, antiemetics, gastrointestinal anti-inflammatory agents, salicylate gastrointestinal anti-inflammatory agents, antacid anti-ulcer agents, gastric acid-pump inhibitor anti-ulcer agents, gastric mucosal anti-ulcer agents, H2-blocker anti-ulcer agents, cholelitholytic agents, digestants, emetics, laxatives and stool softeners, and prokinetic agents; general anesthetics, such as inhalation anesthetics, halogenated inhalation anesthetics, intravenous anesthetics, barbiturate intravenous anesthetics, benzodiazepine intravenous anesthetics, and opiate agonist intravenous anesthetics; hormones and hormone modifiers, such as abortifacients, adrenal agents, corticosteroid adrenal agents, androgens, anti-androgens, immunobiological agents, such as immunoglobulins, immunosuppressives, toxoids, and vaccines; local anesthetics, such as amide local anesthetics and ester local anesthetics; musculoskeletal agents, such as anti-gout anti-inflammatory agents, corticosteroid anti-inflammatory agents, gold compound anti-inflammatory agents, immunosuppressive anti-inflammatory agents, nonsteroidal anti-inflammatory drugs (NSAIDs), salicylate anti-inflammatory agents, minerals; vitamins, such as vitamin A, vitamin B, vitamin C, vitamin D, vitamin E, and vitamin K; and radionuclides such as Yttrium-90, Iodine-131, Samarium-153, Lutetium-177, Astatine-211, Lead-212/bismuth-212, Radium-223, Actinium-225, and Thorium-227.

Additional non-limiting examples of useful therapeutic agents from the above categories include: (1) analgesics in general, such as lidocaine or derivatives thereof, and nonsteroidal anti-inflammatory drugs (NSAIDs) analgesics, including diclofenac, ibuprofen, ketoprofen, and naproxen; (2) opiate agonist analgesics, such as codeine, fentanyl, hydromorphone, and morphine; (3) salicylate analgesics, such as aspirin (ASA) (enteric coated ASA); (4) Hi-blocker antihistamines, such as clemastine and terfenadine; (5) anti-infective agents, such as mupirocin; (6) antianaerobic anti-infectives, such as chloramphenicol and clindamycin; (7) antifungal antibiotic anti-infectives, such as amphotericin b, clotrimazole, fluconazole, and ketoconazole; (8) macrolide antibiotic anti-infectives, such as azithromycin and erythromycin; (9) miscellaneous B-lactam antibiotic anti-infectives, such as aztreonam and imipenem; (10) penicillin antibiotic anti-infectives, such a s nafcillin, oxacillin, penicillin G, and penicillin V; (11) quinolone antibiotic anti-infectives, such as ciprofloxacin and norfloxacin; (12) tetracycline antibiotic anti-infectives, such as doxycycline, minocycline, and tetracycline; (13) antituberculosis antimycobacterial anti-infectives such as isoniazid (INH), and rifampin; (14) antiprotozoal anti-infectives, such as atovaquone and dapsone; (15) antimalarial antiprotozoal anti-infectives, such as chloroquine and pyrimethamine; (16) anti-retroviral anti-infectives, such as ritonavir and zidovudine; (17) antiviral anti-infective agents, such as acyclovir, ganciclovir, interferon alfa, remdesivir, and rimantadine; (18) antifungal topical anti-infectives, such as amphotericin B, clotrimazole, miconazole, and nystatin; (19) antiviral topical anti-infectives, such as acyclovir; (20) electrolytic and renal agents, such as lactulose; (21) loop diuretics, such as furosemide; (22) potassium-sparing diuretics, such as triamterene; (23) thiazide diuretics, such as hydrochlorothiazide (HCTZ); (24) uricosuric agents, such as probenecid; (25) enzymes such as RNase and DNase; (26) antiemetics, such as prochlorperazine; (27) salicylate gastrointestinal anti-inflammatory agents, such as sulfasalazine; (28) gastric acid-pump inhibitor anti-ulcer agents, such as omeprazole; (29) H2-blocker anti-ulcer agents, such as cimetidine, famotidine, nizatidine, and ranitidine; (30) digestants, such as pancrelipase; (31) prokinetic agents, such as erythromycin; (32) ester local anesthetics, such as benzocaine and procaine; (33) musculoskeletal corticosteroid anti-inflammatory agents, such as beclomethasone, betamethasone, cortisone, dexamethasone, hydrocortisone, and prednisone; (34) musculoskeletal anti-inflammatory immunosuppressives, such as azathioprine, cyclophosphamide, and methotrexate; (35) musculoskeletal nonsteroidal anti-inflammatory drugs (NSAIDs), such as diclofenac, ibuprofen, ketoprofen, ketrolac, and naproxen; (36) minerals, such as iron, calcium, and magnesium; (37) vitamin B compounds, such as cyanocobalamin (vitamin B12) and niacin (vitamin B3); (38) vitamin C compounds, such as ascorbic acid; and (39) vitamin D compounds, such as calcitriol.

### BioNVs

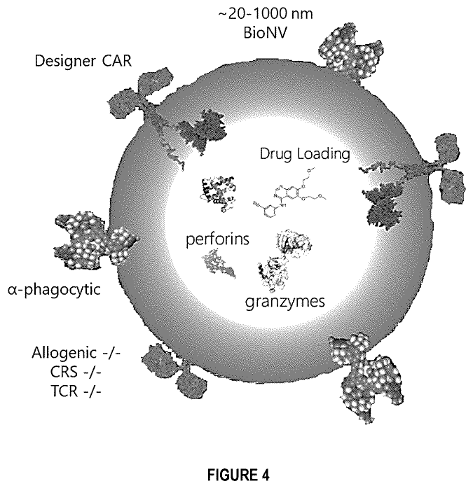

In aspects, the present invention includes BioNVs. As depicted in FIG. 4, BioNVs are approximately 20-1200 nm in size which contain outwardly facing, membrane-embedded CARs, VERRs, and/or viral ligands capable of binding a target molecule. Without wishing to be bound by theory, the biomimetic quality owes to the nanovesicle composition which originates from the plasma membrane of allogeneic, hypoimmunogenic modified cells. In embodiments, BioNVs comprise plasma membrane-derived lipid bilayers, fully encapsulating an aqueous core which can house a variety of cell-derived molecules, including perforins, granzymes, cytokines, etc. In embodiments, the aqueous core of the NVs can further enclose exogenous biologics, fluorescent proteins, tracing dyes, radionuclides, and small molecules, among other therapeutic agents.

To ensure proper directionality of CARs and/or VERRs and/or viral ligands and to eliminate BioNVs lacking CARs and/or VERRs and/or viral ligands, in embodiments, HPLC-based affinity chromatography techniques can be used to select and concentrate only the BioNVs with a sufficient surface concentration of solvent-exposed CARs and/or VERRs and/or viral ligands. HPLC-based affinity chromatography techniques can be used to reduce the concentration of contaminating cell material and NVs which harbor immunogenic cell surface markers, either by positive or negative selection.

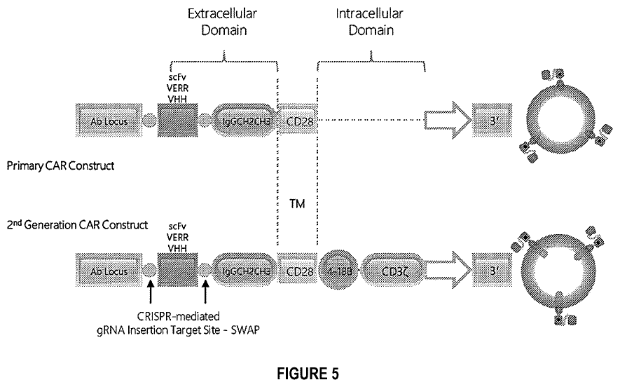

CAR and/or VERR and/or viral ligand constructs may comprise a variety of structural molecules, such as fused with proteins that are typically used in a chimeric antigen receptor (CAR). The structure-function of a prototypical CAR, as depicted in FIG. 5, includes a fusion protein comprising an extracellular (or outwardly facing) binding moiety (e.g., scFv), connected by a hinge peptide (e.g., CH2/CH3 domains from an IgG Fc region, Gly-Gly-Ser peptide linkage, CD28 peptide, CD8a peptide, etc.) to a transmembrane domain (e.g., CD28, CD37, CD4, CD8a, ICOS, etc.), followed by a variety of intracellular signaling domains (e.g. 4-1BB, CD37, CD28, 4-1BB, ICOS, CD27, OX40, etc.). In embodiments, BioNVs lack the intracellular machinery of whole cells and therefore the CAR and/or VERR and/or viral ligand design does not necessitate any intracellular signaling molecules (primary CAR or VERR construct; FIG. 5). In embodiments, the CAR and/or VERR and/or viral ligand construct includes an extracellular CAR and/or VERR and/or viral ligand binding moiety fused with an IgG CH2/CH3 linker to a CD28 transmembrane domain and substantially lacks any intracellular domains or functionality. In embodiments, the CAR and/or VERR and/or viral ligand construct can be a fusion protein with Vp1 AAV and can have the prototypical intracellular domains swapped or otherwise fused to anchor proteins, e.g., PLA2 domain from an AAV, fusion proteins, radionuclide-binding domains, cytoskeletal elements, small molecule transporting domains, etc., which may aid in the fusion to target cells and/or packaging and release of therapeutic payloads.

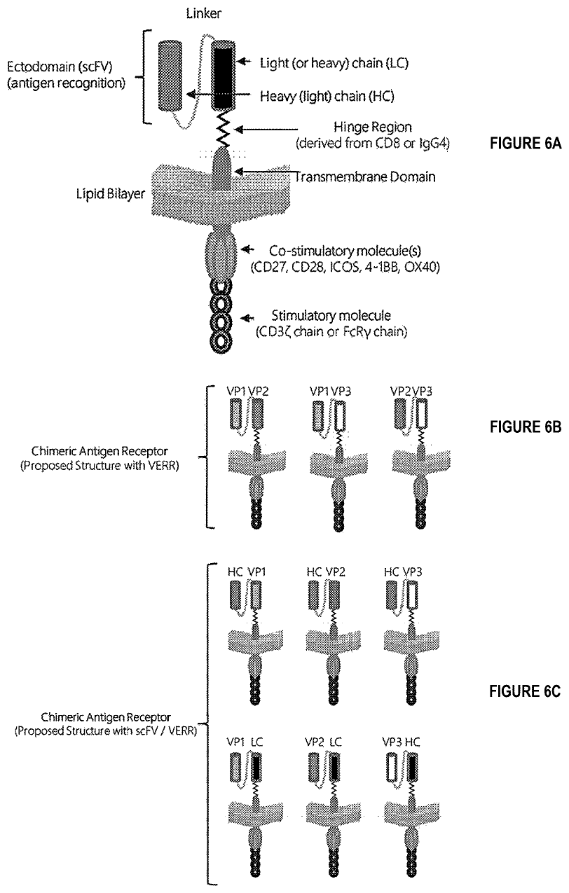

In embodiments, CAR and/or VERR and/or viral ligand antigen-binding molecules comprise a variety of binding moieties, including antibody-based or antibody format binding domains. In embodiments, BioNVs comprise antibody or antibody format binding moieties selected from one or more of a monoclonal antibody, polyclonal antibody, antibody fragment, VERR, viral ligand, Fab, Fab′, Fab′-SH, F(ab′)2, Fv, single chain Fv (scFv), diabody, nanobody, linear antibody, bispecific antibody, multi-specific antibody, chimeric antibody, humanized antibody, human antibody, and fusion protein comprising the antigen-binding portion of an antibody. In embodiments, the CAR and/or VERR and/or viral ligand construct includes binding moieties with a Bispecific T cell Engager (BiTE), viral epitope recognition receptor (VERR), variable heavy chain IgG fragment VHH, VNAR, or through an engineered T-Cell Receptor (TCR). In embodiments, the VERR and/or viral ligand includes and/or targets VP1/VP2/VP3 proteins (see FIG. 6B). in embodiments, the VERR and/or viral ligand is a fusion with a scFv of a heavy chain (HC) or light chain (LC) variable portion (see FIG. 6C).

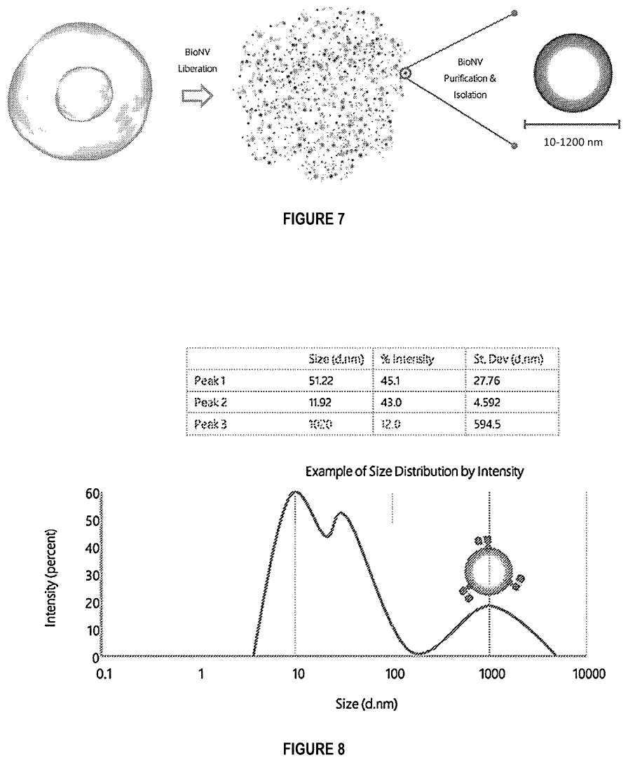

In embodiments, BioNVs are formed by disrupting the cell membranes of engineered iPSCs. In embodiments, the hypo-iPSCs are characterized by a B2M−/−, CIITA−/−, CD47+/+, PD1−/− plasma membrane profile and may be used to generate the present BioNVs (FIG. 7). Hypo-BioNVs can be generated from the parent iPSC cell line via sonication, adaptive focused acoustics technology, French press, extrusion, serial extrusion, cell lysis by detergent, and electroporation, among other methods. In embodiments, serial extrusion is the method used to generate Hypo-BioNVs. Serial extrusion of iPSCs can produce BioNVs that are HLA1/HLA2 negative (hypoimmunogenic) with tgCD47+, exhibiting PD1 resistance elimination.

In embodiments, BioNVs can be analyzed for homogeneity of size by dynamic light scattering (DLS), flow cytometry, mass photometry, among other methods of determining particle size. In embodiments, BioNVs can be filtered for a particle size, or range of sizes, to optimize renal clearance and other clinically-relevant NV properties. FIG. 8 details the range of sizes BioNVs have been observed to adopt. BioNVs can be about 20 nm to 1200 nm in size. BioNVs are about 10 nm in size, about 20 nm in size, about 30 nm in size, about 40 nm in size, about 50 nm in size, about 60 nm in size, about 70 nm in size, about 80 nm in size, about 90 nm in size, about 100 nm in size, about 120 nm in size, about 140 nm in size, about 160 nm in size, about 180 nm in size, about 200 nm in size, about 300 nm in size, about 400 nm in size, about 500 nm in size, about 600 nm in size, about 700 nm in size, about 800 nm in size, about 900 nm in size, about 1000 nm in size, about 1100 nm in size, or about 1200 nm in size. BioNVs range in size from about 10 nm to 20 nm in size, about 20 nm to 30 nm in size, about 30 nm to 40 nm in size, about 40 nm to 50 nm in size, about 50 nm to 60 nm in size, about 60 nm to 70 nm in size, about 70 nm to 80 nm in size, about 80 nm to 90 nm in size, about 90 nm to 100 nm in size, about 10 nm to 100 nm in size, about 100 nm to 200 nm in size, about 200 nm to 400 nm in size, about 400 nm to 600 nm in size, about 600 nm to 800 nm in size, about 800 nm to 1000 nm in size, or about 1000 nm to 1200 nm in size.

iPSC-derived BioNVs, in embodiments, include NVs with an outer plasma membrane leaflet only, an inner plasma membrane leaflet only, and/or both leaflets of a plasma membrane lipid bilayer intact. iPSC-derived NVs, in embodiments, include additional lipid additives (e.g., phosphatidylethanolamine, phosphatidylcholine, phosphatidylinositols, ceramides, lecithin, etc.), non-ionic surfactants (e.g., sorbitan monostearate, octadecylamine, etc.), sterols (e.g., cholesterol, bile salt derivatives, etc.), polyols (e.g., maltodextrin, sorbitol, sucrose, mannitol, etc.) and proteins (e.g., serum albumin, etc.) added for improved physicochemical properties, such as thermal stability and therapeutic payload packaging/release. The amount of cholesterol and the length and saturation of the hydrocarbon chains of the phospholipids can affect the rigidity and the stability of the bilayer, and in turn the capability of the NVs to host and release drugs, biomolecules, and other therapeutic payloads. In embodiments, BioNVs also incorporate zwitterionic lipids and methods of using zwitterionic lipids, for example, as described in US Patent Publication No. US20130216607, the contents of which are herein incorporated by reference in its entirety. Correspondingly, functionalization of the hydrophilic heads of the lipids with polymers or biomolecules can provide additional features to the vesicle surface, thus shaping their interaction with blood components, tissues, and the immune system in vivo.

In embodiments, CAR, VERR, and/or viral ligand targets include a variety of cell surface markers, including markers of cellular infection with a virus. In embodiments, in the context of targeting latently HIV-infected immune cells, sialic acid-binding immunoglobulin-like lectins (Siglecs), such as Siglec-1, is the effective target for the CAR and/or VERR and/or viral ligand binding moiety. Siglecs can be divided into subsets based on sequence and structure similarity, such as CD33-related Siglecs (e.g., Siglec-H, Siglec-5, and Siglec-14) and CD22-related Siglecs. In embodiments, BioNVs are targeted against at least a portion of one or more of Siglec-1, CD2, CD3, CD4, CXCR4, CCR5, CD20, CD25, CD30, CD32a, CD69, CD91, CD160, CD257, LAG-3, CD147, CD231, CEACAM1, PLXNB2, HLA-DR, PD-1, CTLA-4, TIGIT, LAG-3, TIM-3, BTK, Cyclophilin B, Sec62, Rab10, and/or SPCS.

In embodiments, the VERR and/or viral ligand includes a receptor and fusion peptide combined in a single complex. In embodiments, the VERR and/or viral ligand does not contain combined fusion and recognition functionality. In embodiments, the VERR and/or viral ligand has the same cell and/or tissue tropism as the virus from which the VERR and/or viral ligand originates. In embodiments, the VERR and/or viral ligand has altered cell and/or tissue tropism compared to the virus from which the VERR and/or viral ligand originates. In embodiments, the VERR and/or viral ligand facilitates endosomal uptake. In embodiments, the VERR and/or viral ligand facilitates membrane fusion (fusogen).

In embodiments, the VERR and/or viral ligand comprises a protein that targets cell adhesion molecules (CAMs). A majority of viral receptors identified to date are CAMs that function in cell-cell and cell-to-extracellular matrix adhesion and are essential mediators of cellular processes such as development, maintenance of cellular structure, cell signaling, and maintenance and repair of tissues. The family of CAMs includes selectins, cadherins, integrins, and IgSF members. The ubiquitous expression and multifactorial function of CAMs in ligand binding, endocytosis, and signaling provides a multitude of cell targeting mechanisms for BioNVs to engage CAMs. In embodiments, viral ligands include fusions, variants, or portions of the viral ligands of HIV, measles virus, reovirus, rhinovirus, adenovirus, poliovirus, and coxsackievirus B (CVB) (IgSF receptor) that are known to engage CAM receptors.

In embodiments, the BioNVs comprise one of more VERR and/or viral ligand and/or host cell receptor components of Table 1.

TABLE 1
Illustrative viral ligand-cell receptor for VERR and/or viral ligand targeted BioNVs.

| Viral Ligand | Host Cell Receptor |
|---|---|
| JC polyomavirus (JCPγV) | α2,6 SA-containing LSTc receptor |
| Adenovirus glycoprotein | α/β-integrin pairing (αvβ3 and αvβ5) |
| Zika Virus Gas6/PtdSer | AXL receptor tyrosine kinase |
| Influenza glycoproteins hemagglutinin (HA) and | Sialic acid (SA) receptors |
| neuraminidase (NA); H1, H7, H10 | |
| Coronaviral spike protein | MERS-HA receptor; COVID-19-ACE2 receptor |
| Polyomaviruses (SV40, BKPyV, and PyV) | Each utilizes ganglioside receptors containing |
| | α2,3- and α2,8-linked SA |
| Seneca Valley virus (SVV) VP1/VP2/VP3 proteins | Tumor endothelial marker 8 (TEM8) |
| HIV ENV (gp120/gp41) | CD4, CXCR4, and/or CCR5 |
| Rabies Virus glycoprotein (RVG) | Neurotrophin receptor (p75); nicotinic |
| | acetylcholine receptor (nAChR); neuronal cell |
| | adhesion molecule (NCAM) |

In embodiments, the VERR and/or viral ligand comprises a human cytomegalovirus (HCMV) full-length gB protein, fusion, variant, or portion where the extracellular domain (ECD) of gB is membrane-anchored using the transmembrane and cytoplasmic domains of the vesicular stomatitis virus (VSV) G protein. (see e.g., Kirchmeier M, et al. “Enveloped virus-like particle expression of human cytomegalovirus glycoprotein B antigen induces antibodies with potent and broad neutralizing activity.” Clin Vaccine Immunol. 2014 February; 21 (2): 174-80). In embodiments, the VERR and/or viral ligand comprises targeting both the gB and pp65 antigens. The gB and pp65 antigens can be expressed by epithelial cells, macrophages, and T lymphocytes. Alternatively, a combination of gB, pentamer, and pp65 or G-protein coupled receptor (GPCR) homolog, US28, can be used in embodiments (Perotti, M., and Laurent P. “Virus-Like Particles and Nanoparticles for Vaccine Development against HCMV.” Viruses vol. 12,1 35. (2019), doi:10.3390/v12010035).

In embodiments, the VERR and/or viral ligand comprises a viral protein that targets integrins, which have been demonstrated as receptors for reovirus, rotavirus, adenovirus, West Nile virus (WNV), human metapneumovirus (hMPV), foot-and-mouth disease virus (FMDV), and herpes simplex virus (HSV), as well as human cytomegalovirus HCMV and human herpesvirus-8. IgSF members have emerged as receptors for a wide range of viruses including enveloped and nonenveloped viruses, including reovirus, adenovirus, coxsackievirus, rabies virus, measles virus, and HIV.

In embodiments, the VERR and/or viral ligand comprises a viral protein that targets PtdSer receptors, including T-cell immunoglobulin and mucin domain (TIM) and TYRO3, AXL, and the MERTK family of receptor tyrosine kinases (TAMs), both of which been shown to serve as receptors for many enveloped viruses. PtdSer receptors have been reported to mediate viral entry of a number of enveloped viruses including the filoviruses EBOV and Marburgvirus (MARV); flaviviruses such as WNV, Dengue virus (DENV), and Zika virus (ZIKV), arenavirus such as Lassa Virus, and poxvirus such as vaccinia virus. AXL is also an entry receptor for DENV. AXL-mediated entry of both ZIKV and DENV is mediated through Gas6, an AXL ligand that binds directly to phosphatidylserine and binds to AXL.

In embodiments, the VERR and/or viral ligand comprises a viral protein that targets the IgSF member CD4 as the primary receptor, e.g., as is the case with HIV, which also requires specific coreceptors CXCR4 and CCR5. In embodiments, the VERR and/or viral ligand can include the HIV envelope (ENV) glycoprotein which interacts with CD4 to mediate specific interactions between ENV and CD4 that would facilitate entry into immune cells. However, initial interactions between ENV and host cells occur via nonspecific cellular receptors including heparan sulfate proteoglycans or specific receptors such as α4β7 integrin or the innate immune receptor DC-SIGN. ENV is a trimeric protein composed of gp120 and gp41, and gp120 mediates binding to CD4 through conserved domains leading to conformational changes within gp120 and CD4. Following CD4 interactions, the ENV viral ligand binding to the chemokine coreceptors CXCR4 or CCR5 can be used to specifically target BioNVs to macrophages and CD4+ T cells, respectively. The BioNV can additionally use HIV gp41 for targeting appropriate coreceptor interactions.

In embodiments, BioNVs are targeted to T cells by use of sialyllactose from gangliosides which can serve as the viral attachment factor for Sialyl glycan receptor expression on T cell subsets for targeting to immune cells, for example, via hemagglutinin protein selected from H1, H7 and H10 from Influenza A, or H1N1, H3N2, H7N9, and H10N8, among others. The BioNV may be targeted to a cell via a viral fusogen, viral glycoprotein which facilitates fusion of the NV to the cell plasma membrane.

Those skilled in the art will understand that VERRs/viral ligands can be designed to target cell surface markers of any cell subset of interest and the appropriate methods for optimization.

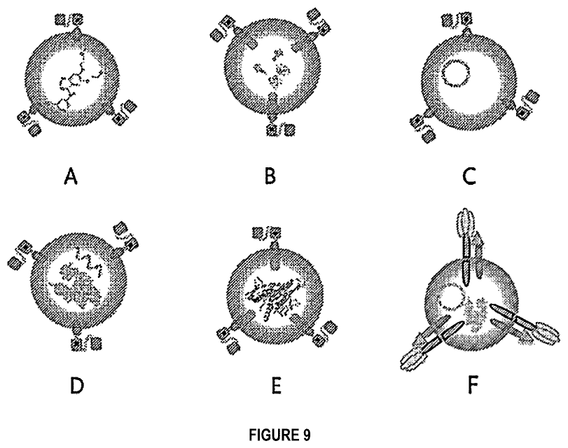

In embodiments, BioNVs are configured to encapsulate a variety of therapeutic payloads. As FIG. 9 illustrates, in embodiments, primary targeted BioNVs can be used to deliver small molecule therapeutic payloads (A). In embodiments, second generation (or 3^{rd} or 4^{th} gen) CAR and/or VERR and/or viral ligand-containing BioNVs derived from activated lymphocytes can contain cytokines and other cytotoxic peptides (B). In embodiments, BioNVs can be formatted to encapsulate and deliver plasmid DNA, for example, to express gene editing nucleases and gRNA in target cells (C). Alternatively, or additionally, in embodiments, BioNVs can encapsulate the nucleases and gRNA (D). In embodiments, targeted second generation (or 3^{rd} or 4^{th} gen) BioNVs can be designed to encapsulate and deliver additional therapeutic proteins or peptides of interest (E). In embodiments, HIV gp120/gp41 receptor-ligand coated BioNVs can target lymphocytes (among other cellular targets when the gp120 epitopes are altered) to deliver biologics or chemical payloads into the cytoplasm of target cells (F).

In embodiments, BioNVs deliver a gene editing payload comprising a transactivating response region (TAR) loop system. In embodiments, the BioNV encapsulates a plasmid which expresses a gene editor and contains a TAR loop sequence between the 5′ end of the promoter and the gene editor/guide cassette and acts as a barrier, blocking transcription. In embodiments, transcription will only trigger in cells that are infected and contain the HIV Tat protein. In embodiments, the Tat protein binds to the TAR Loop, relaxes it, and frees the promoter for transcription, thereby expressing the editor and its guides.

In embodiments, the BioNVs are CD34+, or derived from CD34+ cells, such as human CD34+ cord blood. CD34+ cord blood-derived cell lines may serve as a base cell line for BioNV development, production, and manufacturing for the delivery of gene editing therapeutics. CD34+ cord blood-derived hypo-immunogenic cell line has been experimentally confirmed for the low expression of HLA 1/2 and overexpression of CD47 (Deuse T. et al. Hypoimmunogenic derivatives of induced pluripotent stem cells evade immune rejection in fully immunocompetent allogeneic recipients. Nat Biotechnol. 2019 March; 37(3):252-258).

In embodiments, the BioNVs and/or compositions comprising BioNVs are administered in combination with one or more additional compounds. In embodiments, the BioNVs are pretreated with one or more additional compounds, for example prior to administration to a subject.

In embodiments, BioNVs are modular and allogeneic (off-the-shelf) due to the lack of immunogenicity from engineered iPSCs. In embodiments, the lack of whole cell signaling components allows BioNVs to be easily tunable for target specificity and resistance to immunosuppressive signals. In embodiments, BioNVs lack the genetic elements that contribute to runaway cytokine storms, minimizing patient risk of cytokine release syndrome (CRS). In embodiments, the amounts of active cytokine, perforin, granzymes, interferon, interleukins, etc., encapsulated within the BioNV are regulated during upstream (pre-BioNV derivation) cellular processes. In embodiments, BioNVs are derived from cells capable of crossing biological barriers and/or viral receptors known for facilitation crossing.

Without wishing to be bound by theory, BioNVs generated from iPSC engineered allogeneic base cell lines represent immune invisible BioNVs which have the potential for multi-dosing. BioNV antibody-mediated neutralization is minimized, and immune cell-mediated clearance is evaded (T cell and macrophage). In embodiments, BioNVs do not contain viable genetic material from the cells they were derived to cause CRS or teratoma. In embodiments, increased expression of certain cytokines encapsulated within a BioNV can recruit natural T cells. In embodiments, BioNVs can be derived from modified cell types with or without barrier penetrating ligands to further control activity post-infusion.

### Methods of BioNV Formation

In embodiments, the modified cell is a hypoimmunogenic cell derived from iPSCs that have been engineered to have reduced or ablated expression and/or activity of immunogenic proteins and/or express or have increase expression of immunoprotective proteins. In embodiments, iPSCs are reverted from a somatic state using microRNA technology in lieu of small molecule trans-activators. The use of microRNA provides a tighter differentiation system and that results in higher quality iPSCs. Without wishing to be bound by theory, these high quality iPSCs are less prone to expression dampening (of post-engineered proteins, such as CD47) and genetic drift, and possess higher culture splitting qualities/quantities (the cultures can be divided more times than other methods before cellular integrity issues occur).

In embodiments, BioNVs derived from iPSC-derived hypoimmunogenic cells which retain the functionality from the hypoimmunogenic cell, for example and without limitation, the ability to cross the blood-brain barrier, such as is the case of macrophages/monocytes, or tissue-specific factors such as is the case in cardiomyocytes, hepatocytes, etc.

In embodiments, allogeneic iPSCs have their MHC class I and MHC class II complexes disrupted by knocking out critical proteins involved in their expression, for example, B2M which is a serum protein found in association with the MHC class I heavy chain on the surface of nearly all nucleated cells that is involved in the presentation of peptide antigens to the immune system.

In embodiments, once the B2M knock-out (KO), CIITA KO, IL-6 KO, and CD47tg knock-in (KI) is engineered into the iPSC, the TRAC and TRBC genes can be knocked out. In embodiments, only one gene for each is knocked out rather than both of on the separate alleles. In embodiments, the TRAC and TRBC genes can be knocked out as described herein. The purpose of knocking out the TRAC and TRBC genes is to eliminate the T-cell receptors. In embodiments, the modified cell is differentiated to a T-cell subset which lacks T-cell receptors to derive the BioNVs. Genetically modifying the cells to substantially lack TCRs reduces the chances for a competing ligand to the CAR and/or VERR and/or viral ligand construct that can target non-specifically to alternate tissues. Therefore, in embodiments, the TCR genes are knocked-out as a strategy to reduce off-target effects of the BioNVs. In embodiments, TRAC/TRBC knock-outs decrease the likelihood of CRS, as well as BioNV toxicity, generally.

In embodiments, the modified cell is expanded after engineering; any small-scale expansion or large-scale feeder system expansion methods known in the art can be used.

In embodiments, after the B2M KO, CIITA KO, IL-6 KO, CD47tg KI, and an IL-2 promoter-driven green fluorescence protein (GFP) (IL-2p GFP) reporter is constructed, the CAR and/or VERR and/or viral ligand constructs can be integrated/engineered into the cell. In embodiments, the CAR and/or VERR and/or viral ligand constructs can be knocked-in to the TRAC/TRBC genes, simultaneously knocking-out the remaining TRAC/TRBC genes, resulting in a cell that is CAR and/or VERR and/or viral ligand+ and TRAC/TRBC−/−. In embodiments, the CAR and/or VERR and/or viral ligand construct can be knocked-in to the TRAC/TRBC gene location on both loci simultaneously, resulting in a cell that is CAR and/or VERR and/or viral ligand+/+ and TRAC/TRBC−/−.

In embodiments, once the B2M KO, CIITA KO, IL-6 KO, CD47tg KI, IL-2p GFP KI, and CAR and/or VERR and/or viral ligand modified cells (e.g., iPSCs) are engineered, the Immunological Synapse (IS) quality is measured between the CAR and/or VERR and/or viral ligand recognition domains and the biomarker. In embodiments, the quality of the IS of BioNVs can be directly related to efficacy in whole cell therapies.

In embodiments, the BioNVs, or the hypoimmunogenic cell derived therefrom, comprises a nucleic acid encoding GFP (among other fluorescence proteins). In embodiments, once the B2M KO, CIITA KO, CD47tg KI, IL-6 KO, TRAC/TRBC single KOs are engineered into the iPSC, a GFP molecule is engineered into the modified cell line. In embodiments, this serves as the control cell line. In embodiments, the non-control cell line (the therapeutic cell line) does not have GFP. In embodiments, the nucleic acid encoding GFP is operably linked to a promoter from one or more of IL-2, perforin, granzyme, alarmin, TNF, INF, a combination thereof, and/or any other cell-specific gene or reporter gene. The IL-2 promoter is constitutively activated when lymphocytes are broadly/globally activated from various stimuli. In embodiments, a more focused activation/repression (regulation) is used. In embodiments, the IL-2p GFP reporter gene serves as an indicator for the degree of broad/global activation of the cell (as part of the BioNV derivation process). In embodiments, the GFP signal, coupled with immunoblot analysis of cytokine levels (such as perforins, granzymes, alarmins, TNFs, and INFs) allows efficient regulation of the degree of broad/global activation of a lymphocyte when exposed to activating antigens. In embodiments, GFP is used to compare the degrees of activation between manufacturing lots and ensure consistency for therapeutic development.

In embodiments, the hypoimmunogenic cells are CD34+, or derived from CD34+ cells, such as human CD34+ cord blood. In embodiments, CD34+ cord blood-derived cell lines may serve as a base cell line for BioNV development, production, and manufacturing for the delivery of gene editing therapeutics. In embodiments, a CD34+ cord blood-derived hypoimmunogenic cell line has been experimentally confirmed for the low expression of HLA 1/2 and overexpression of CD47 (Deuse et al. “Hypoimmunogenic derivatives of induced pluripotent stem cells evade immune rejection in fully immunocompetent allogeneic recipients.” Nat. Biotechnol. Vol. 37, No. 3, 2019: pp. 252-258. doi:10.1038/s41587-019-0016-3).

In embodiments, the hypoimmunogenic cell can be engineered using multiple hypoimmunogenic engineering techniques, for example, as described in Deuse et al., Han et al., Xu et al., and Harding et al., and also as described in published U.S. patent applications US20190376045, 20190376045, 20210308183, and 20210292715 to Deuse, US20210161971 to Nagy, US20180141992 to Strominger, and Published European patent application 3693384 to Poirot, each of which is incorporated by reference herein in their entirety, (Han X, et al. “Generation of hypoimmunogenic human pluripotent stem cells.” PNAS. Vol. 116, No. 21 2019: pp. 10441-10446. doi:10.1073/pnas. 1902566116), (Xu H, et al. “Targeted Disruption of HLA Genes via CRISPR-Cas9 Generates iPSCs with Enhanced Immune Compatibility.” Cell Stem Cell. Vol. 24, No. 4, 2019: pp. 566-578. doi: 10.1016/j.stem.2019.02.005), and (Harding et al., “Induction of long-term allogeneic cell acceptance and formation of immune privileged tissue in immunocompetent hosts.” BioRxiv 716571 [Preprint], Jul. 30, 2019. doi:10.1101/716571).

In embodiments, BioNVs are derived from cells which have eliminated HLA genes that encode the MHC membrane glycoproteins that confer immune reactions associated with GVHD rejections. The HLA gene clusters can be divided into three categories: 1) the MHC Class I pathway, 2) the MHC Class II pathway, and 3) the MHC Class III pathway. Only the MHC Class I and II pathways express the protein complexes elicit an immune response in GVHD, whereas MHC Class III complexes are not involved in immunization activities.

The elimination of the MHC classes of protein complexes can trigger natural killer (NK) cells and macrophages into an active clearance mode where the cells are subsequently destroyed. To avoid this kill mechanism, in embodiments, the addition of a CD47 isoform 2 transmembrane molecular protein tag can be engineered into the cell membrane of the modified cell to avoid NK cell and macrophage-mediated kill responses, for example, as described in Willingham et al., Deuse et al., and Han et al. (Willingham S B, et al. “The CD47-signal regulatory protein alpha (SIRPa) interaction is a therapeutic target for human solid tumors.” PNAS. Vol. 109, No. 17, 2012: pp. 6662-7. doi: 10.1073/pnas. 1121623109). In embodiments, cells can be engineered to use additional mechanisms to prevent these responses such as those described in: 1) the CD24 transmembrane molecular protein tags (for example as performed in Zhao et al.) (Zhao W, et al. “Strategies for Genetically Engineering Hypoimmunogenic Universal Pluripotent Stem Cells.” iScience. Vol. 23, No. 6, 2020:101162. doi: 10.1016/j.isci.2020.101162), 2) the membrane-bound surfactant protein-D (SP-D) (for example as performed in Jiaravuthisan et al.) (Jiaravuthisan P, et al. “A membrane-type surfactant protein D (SP-D) suppresses macrophage-mediated cytotoxicity in swine endothelial cells.” Transpl Immunol. Vol. 47, 2018: pp. 44-48. doi:10.1016/j.trim.2018.02.003), and 3) the molecular PD-L1 tag for prevention of T-cell responses. In embodiments, a BioNV derived from an ‘activated’ cell would encapsulate and/or release perforin and/or granzyme, resulting in targeted cell death. In embodiments, the activated cell would generate perforin and/or granzyme to be packaged into the BioNV. In embodiments, hypoimmunogenic cells that are to be activated would not express PD-L1 to avoid the resultant BioNV from being targeted to PD-1 on T-cells. In embodiments, this reduces the likelihood of releasing perforin and/or granzyme, resulting in unwanted T-cell death. In embodiments, PD-L1 is overexpressed in BioNVs derived from a cell that has not been activated and is not loaded with apoptotic cytokines. In embodiments, hypoimmunogenic cells that are to be activated have PD-L1 downregulated, knocked-out, or otherwise silenced. In embodiments hypoimmunogenic cells that are not to be activated have PD-L1 upregulated, i.e. for BioNVs used for gene editor delivery. In embodiments, CD47 can be utilized in genetically engineered iPSCs for immune tolerance to innate immune cells, for example, such as in Chhabra et al., Han et al., and Jaiswal et al. (Chhabra A, et al. “Hematopoietic stem cell transplantation in immunocompetent hosts without radiation or chemotherapy.” Sci Transl Med. Vol. 8, No. 351, 2016: 351ra105. doi:10.1126/scitranslmed.aae0501) and (Jaiswal S, et al. “CD47 is upregulated on circulating hematopoietic stem cells and leukemia cells to avoid phagocytosis.” Cell. Vol. 138, No. 2, 2009: pp. 271-85. doi:10.1016/j.cell.2009.05.046). In embodiments, cells can be modified as described in U.S. Pat. No. 8,562,997 to Jaiswal, et al., which is incorporated by reference herein in its entirety.

In embodiments, some approaches can be used which do not entirely knock-out all HLA genes, for example, as performed in Xu et al. and Han et al., which only knock-out the HLA genes that are highly associated with an immune response, leaving intact the HLA genes that dampen a macrophage or NK response (e.g., HLA-E, HLA-F, and HLA-G). In embodiments, this approach does not require the addition of a CD47 tag; the modified cell can be engineered to generate BioNVs with or without CD47.

In embodiments, methods improve upon the approaches of hypoimmunogenicity of Table 2.

TABLE 2
Three methods of modification of cells using the HLA knockout combined with a CD47 isoform
2 tag and a PD-L1 transmembrane tag (Zhao, et al.) (Gornalusse GG, et al. “HLA-E-expressing
pluripotent stem cells escape allogeneic responses and lysis by NK cells.” Nat Biotechnol.
Vol. 35, No. 8, 2017: pp. 765-772. doi: 10.1038/nbt.3860.).

| | | | | | | | | | |
|---|---|---|---|---|---|---|---|---|---|
| Source | HLA-A | HLA-B | HLA-C | HLA-E | HLA-F | HLA-G | HLA-II | CD47 | PD-L1 |

| Gornalusse, | Knockout | + | Knockout | | | |
|---|---|---|---|---|---|---|
| 2017 | | | | | | |
| Deuse, | B2M Knockout | | | CIITA KO | + | |
| 2019 | | | | | | |
| Xu, | Knockout | | | CIITA KO | | |
| 2019 | | | | | | |
| Han, | Knockout | | | CIITA KO | + | + |
| 2019 | | | | | | |

In embodiments, developing the allogeneic modified cell involves the removal of MHC Class I and MHC class II protein complexes through the disruption of certain HLA genes, or a B2M knockout, followed by knocking out the CIITA gene. In embodiments, the knockouts can be performed using CRISPR gene editing approaches, due to their rapid mechanism of action. In embodiments, the knockouts are performed using Zinc Finger Nucleases (ZFNs) and/or TALENS. In embodiments, Cre/Lox recombinase systems are used to generate the modified cell. In embodiments, RNA silencing (RNAi, shRNA, microRNA, CRISPR Cas13a-d, etc.) is used to generate the modified cell.

In embodiments, the methods of developing the allogeneic, hypoimmunogenic modified cell is distinct from the methods of creating allogenicity of Harding et al. In embodiments, in lieu of deleting the MHC class I/II genes and running the risk of preventing long-term acceptance by the recipient, the Harding et al. method includes an alternate approach based on immune escape mechanisms that occurs in nature. In embodiments, the method relies on the Harding et al. biomimicry based on the horizontally transmitted cancer, devil facial tumor disease (DFTD) type 2, that is predominant in Tasmanian devils. In embodiments, developing the allogeneic modified cell can include expression or increased includes overexpression of the immunomodulatory proteins CCL21, PD-L1, FasL, SerpinB9, H2-M3, CD47, CD200, and/or MFG-E8 to protect cell derivatives from long-term immune rejection in mice (and humans), without the deletion of MHC class I/II proteins. In embodiments, the modified cell expresses one or more of the proteins shown in Table 3, including any splice variant and/or isoform of any of the indicated proteins (e.g., CD200 splice variants). In embodiments, this system can be used to interfere with the activity of antigen presenting cells (APCs), macrophages, NK cells, and T-lymphocytes. In embodiments, the modified cell lines can also contain the safe-cell system developed by Liang et al., where cell division genes are linked to a suicide gene to prevent runaway teratomas leading to cancers (Liang Q, et al. “Linking a cell-division gene and a suicide gene to define and improve cell therapy safety.” Nature. Vol. 563, No. 7733, 2018: pp. 701-704. doi:10.1038/s41586-018-0733-7).

In embodiments, methods improve upon the approaches of hypoimmunogenicity of Table 3.

TABLE 3
Expression or increased expression of illustrative
proteins for creating allogeneic modified cells.

| Gene | Type of protein | Illustrative Immunomodulatory Function |
|---|---|---|
| Ccl2 | Chemokine | Recruitment of activated Dendritic Cells (DCs) |
| PD-L1 | Check Point Inhibitor | Shuts Down Effector T-cell Responses |
| (non-activated | | |
| cells and/or | | |
| BioNVs) | | |
| FASL | Transmembrane Signal | Induces T-cell Apoptosis |
| H2-M3 | Antigen Presentation - MHC-like | Inhibits NK cells |
| SERPINB9 | Inhibitory Chemokine | Blocks granzyme B function |
| | (Serine Protease Inhibitor) | |
| Cd47 | Transmembrane SIRP-αR | Blocks phagocytosis |
| | ligand | |
| Cd200 | Type 1 Membrane Glycoprotein | Prevents macrophage and granulocyte |
| | | activation |
| Mfg-e8 | Chemokine (attractant) | Macrophage recruitment to anti-inflammatory |
| | | profile site |

In embodiments, modified (e.g., hypoimmunogenic) cells from which BioNVs are derived are engineered to have knock-outs of one or more of HLA-A, HLA-B, HLA-C, HLA-E, HLA-G, HLA-F, CIITA, IL-6, IL-4, IL-10, IL-16, TRAC, TRBC, SerpinB9, and/or any combination thereof; and knock-ins of one or more of CCL2, PD-L1 (in BioNVs derived from non-activated cell sources), CTLA-4, H2-M3, CD24, CD47 (minus the 3′ UTR region or an alternate 3′ UTR region that does not contain binding sites for the inhibitory microRNAs), MFG-E8, CD200, and/or any combination thereof.

In embodiments, BioNVs are generated from a modified cell with one or more of the modifications of Table 4.

TABLE 4
Illustrative engineered cell expression profile for BioNV formation for human use
(Fife BT and Bluestone JA. “Control of peripheral T-cell tolerance and autoimmunity
via the CTLA-4 and PD-1 pathways.” Immunol Rev. Vol. 224, 2008: pp. 166-82.
doi: 10.1111/j.1600-065X.2008.00662.x.) and (Rong Z, et al. “An effective approach
to prevent immune rejection of human ESC-derived allografts.” Cell Stem Cell.
Vol. 14, No. 1 2014: pp. 121-30. doi: 10.1016/j.stem.2013.11.014.).

| Knockouts | Knock-ins (inducible or constitutive over-expression) |
|---|---|
| HLA-A | CCL2 |
| HLA-B | PD-L1 (non-activated cells and/or BioNVs) and/or CTLA-4 (Fife et |
| | al. and Rong et al.) |
| HLA-C | H2-M3 |
| HLA-E or HLA-G (but not both) | CD24 or CD47 or CD47 tethered to CD24 |
| HLA-F | MFG-E8 |
| CIITA | CD200 for non-immunomodulatory functions only (such as liver |
| IL-6 | cell regeneration). These cells will not contain a CD47 tag. |
| IL-4, IL-10, or IL-16 (optional) | SerpinB9 |
| TRAC | (where granzyme is not used) |
| TRBC | |
| SerpinB9 | |
| (where granzyme is used) | |

In embodiments, inactivation/activation of genes is controlled by inducible promoters throughout the differentiation, activation, and manufacturing process for BioNVs. In embodiments, disruption of MHC, TCR, and cytokine release syndrome (CRS) genes produce allogeneic iPSCs which are −/− CRS and −/− TCR, leading them to have plasma membranes which exhibit hypoimmunogenic properties upon infusion into a subject. CRS genes implicated in the pathogenesis of CRS include IL-6, IL-10, IFN-γ, monocyte chemoattractant protein 1 (MCP-1), granulocyte-macrophage colony-stimulating factor (GM-CSF), among other cytokines, including tumor necrosis factor (TNF), IL-1, IL-2, IL-2-receptor-α, and IL-8. In embodiments, one or more of these genes is inactivated, e.g., in a cell from which the BioNVs are derived.

In embodiments, BioNVs are formed by disrupting the cell membranes of engineered iPSCs. In embodiments, the hypo-iPSCs are characterized by a B2M−/−, CIITA−/−, CD47+/+, PD1−/− plasma membrane profile and may be used to generate BioNVs. Hypoimmunogenic BioNVs can be generated from the parent iPSC cell line via sonication, adaptive focused acoustics technology, French press, extrusion, serial extrusion, cell lysis by detergent, and electroporation, among other methods. In embodiments, serial extrusion is the method used to generate hypoimmunogenic BioNVs. In embodiments, serial extrusion of iPSCs can produce BioNVs that are HLA1/HLA2 negative (hypoimmunogenic) with tgCD47+, exhibiting PD1 resistance elimination.

In embodiments, genetic engineering of iPSCs includes gene-editing techniques such as CRISPR-based gene editing systems, zinc finger nucleases (ZFNs), transcription activator-like effector nuclease (TALEN), meganucleases, among other gene editing methods, for the purpose of generating allogeneic/hypoimmunogenic iPSCs and/or for CAR and/or VERR and/or viral ligand cassette integration. In embodiments, the genetic engineering of iPSCs can refer to the decrease or ablation of transcription of any genetic element; likewise, genetic engineering of iPSCs can refer to the increase in the expression of or the knock-in of any genetic element, including both endogenous and exogenous genetic elements.

For example, in embodiments, stable cell integration (safe harbor genetic location) in iPSCs can be controlled by implementing a Tet-regulated CRISPRa+targeted 3× transcription factor targeted gRNA system. The CRISPR activation system for three upstream transcription factors can trigger a signal cascade event that enhances the production of CARs and/or VERRs and/or viral ligands that have replaced endogenous antibody ORFs at designated locus (loci). This system can be ‘tunable’ by including a Tet-regulated promoter, allowing for the ability to vary the concentrations of CARs and/or VERRs and/or viral ligands on the surface of the cell. Next, stable cell replacement of CDRs and heavy and light antibody regions with CARs and/or VERRs and/or viral ligands cassettes can be achieved via Cpf-1 directed homology directed repair (HDR). Finally, the stably integrated CAR and/or VERR and/or viral ligand cassette can contain flanking gRNA binding sites which allow the scFV or VERR (among other antibody formats) to be repeatedly swapped or altered for rapid and consistent insertion of a desired sequence.

In embodiments, the allogeneic and hypoimmunogenic properties of the iPSCs can be further improved by inducing overexpression of immunoprotective molecules. For example, and without limitation, overexpression of CD47—among other cell surface integrins—can decrease the kinetics of macrophage depletion of BioNV products from the blood. In embodiments, additionally, allogeneic and hypoimmunogenic properties of iPSCs can be improved by expression of α- and β-phagocytic integrins. In embodiments, overexpression of similar immunogenically protective cell surface markers which signal to leukocytes can be performed as a strategy to increase the half-life of BioNVs post-infusion.

In embodiments, iPSCs are genetically engineered for CAR and/or VERR and/or viral ligand cassette integration. CAR and/or VERR and/or viral ligand cassette integration can include both integrative and non-integrative transgene insertion. Non-limiting examples of non-integrative transgene insertion include mRNA, non-integrative lentivirus, and endonuclease-targeted methods. Integrative CAR and/or VERR and/or viral ligand cassette insertion methods include stable retroviral vector insertion and transposase-based integration systems. Stable CAR and/or VERR and/or viral ligand cassette transduction can be achieved, for example, using retroviral vectors which can enable iPSCs to maintain the genetic element encoding the CAR and/or VERR and/or viral ligand throughout differentiation, expansion, and activation. In embodiments, clinical-grade, stable transduction of CAR and/or VERR and/or viral ligand cassettes into T cells has been achieved for similar CAR cassettes, for example, in brexucabtagene autoleucel (Tecartus®, Kite Pharma Inc.) and axicabtagene ciloleucel (Yescarta®, Kite Pharma Inc.) using GRV vectors, while tisagenlecleucel (Kymriah®, Novartis International AG) is transduced using a lentiviral vector (Labbé, R. P., et al. “Lentiviral Vectors for T Cell Engineering: Clinical Applications, Bioprocessing and Future Perspectives.” Viruses 13 (2021); 1528. doi:10.3390/v13081528).

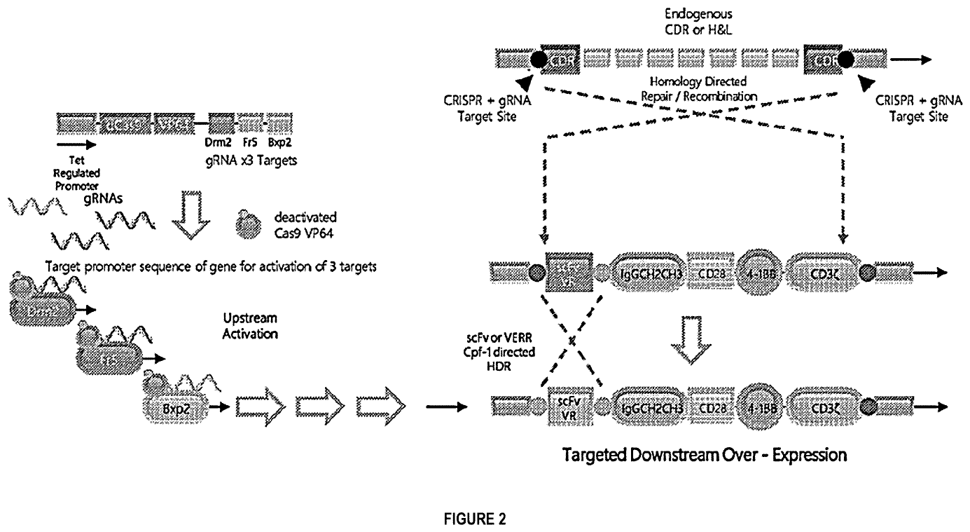

In embodiments, the concentration of the CAR and/or VERR and/or viral ligand on the surface of the iPSC base cell line, or any downstream differentiated cell (and the resulting BioNVs), can be regulated using a variety of transcription control elements, such as a tetracycline on/off promoter (or similar drug-regulated promoters) to drive the expression of a CRISPR activation/gRNA (CRISPRa) system (FIG. 2). The CRISPRa system can then activate the antibody-regulating transcription factors, for example, Drm2, Fr5, and Bxp2, which regulate the expression of an engineered CAR and/or VERR and/or viral ligand cassette that has been integrated at the site of an antibody locus (where the antibody genes have been replaced). Additionally, a similar transcription control element can be provided to control overexpression of genes (e.g., CD47), drive genes controlling differentiation, etc., at defined manufacturing stages.

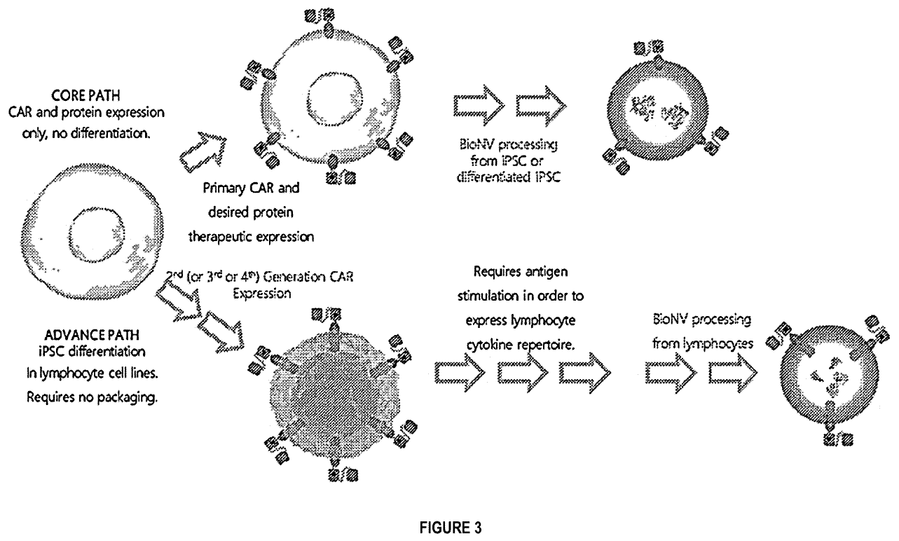

In embodiments, CAR and/or VERR and/or viral ligand expression is initiated in the modified cells with or without differentiation. As FIG. 3 demonstrates, in embodiments, primary CAR and/or VERR and/or viral ligand expression can be performed in iPSCs without differentiation (Core Path) to obtain BioNVs lacking cell surface markers from differentiated cell subsets. Alternatively, iPSCs can be differentiated into a lymphoid or myeloid lineage cell prior to initiating primary CAR and/or VERR and/or viral ligand expression (Advanced Path) for expression of cell surface markers from select immune cell types.

Subjects and/or Animals

In embodiments, the subject and/or animal is a mammal, e.g., a human, mouse, rat, guinea pig, dog, cat, horse, cow, pig, rabbit, sheep, or non-human primate. In embodiments, the subject and/or animal is a non-mammal, for example, a zebrafish. In embodiments, the subject and/or animal is a transgenic animal comprising a fluorescent cell, such as, for example, an RPE cell and/or an immune cell with GFP. In embodiments, the subject and/or animal is a human. In embodiments, the BioNVs originate from fluorescently-tagged cells and/or are packaged with fluorescently-tagged proteins or tags (with e.g., GFP). In embodiments, the human is a pediatric human, human adult, geriatric human, an infant or child. In other embodiments, the human is referred to as a patient.

In embodiments, the method of treatment includes administering to a human who has an age in a range of from about 0 months to about 6 months old, from about 6 months to about 12 months old, from about 12 months to about 18 months old, from about 18 months to about 36 months old, from about 1 year to about 5 years old, from about 5 years to about 10 years old, from about 10 years to about 15 years old, from about 15 years to about 20 years old, from about 20 years to about 25 years old, from about 25 years to about 30 years old, from about 30 years to about 35 years old, from about 35 years to about 40 years old, from about 40 years to about 45 years old, from about 45 years to about 50 years old, from about 50 years to about 55 years old, from about 55 years to about 60 years old, from about 60 years to about 65 years old, from about 65 years to about 70 years old, from about 70 years to about 75 years old, from about 75 years to about 80 years old, from about 80 years to about 85 years old, from about 85 years to about 90 years old, from about 90 years to about 95 years old or from about 95 years to about 100 years old.

In embodiments, the subject is a non-human animal, and therefore the invention pertains to veterinary use. In embodiments, the non-human animal is a household pet. In embodiments, the non-human animal is a livestock animal.

In embodiments, sera and/or immune cells are evaluated and/or effected. In embodiments, immune cells include cells of a subject's and/or animal's innate immune system. In embodiments, such cells include, but are not limited to NK cell, monocyte, DC, B cell, macrophage, CD4+ T cell, and CD8+ T cell. In various embodiments, the invention provides for detecting a presence, detecting an absence, or measuring an amount of viral cDNA or RNA in a sample originating from a subject.

### Kits

The disclosure, in embodiments, provides kits that can simplify the administration of any agent described herein. An exemplary kit of the invention comprises any agent described herein in unit dosage form. In embodiments, the unit dosage form is a container, such as a pre-filled syringe, which can be sterile, containing any agent described herein and a pharmaceutically acceptable carrier, diluent, excipient, or vehicle. In embodiments, the kit further comprises a label or printed instructions instructing the use of any agent described herein. In embodiments, the kit also includes a lid speculum, topical anesthetic, and a cleaning agent for the injection surface. In embodiments, the kit further comprises one or more additional agents described herein.

In aspects, the present invention includes a syringe comprising one or more compositions of the present invention. In embodiments, the syringe is prefilled with a volume of the composition. In embodiments, the syringe is prefilled in a volume of about 1 mL to about 10 mL. In embodiments, the syringe is prefilled in a volume of about 10 mL, about 9 mL, about 8 mL, about 7 mL, about 6 mL, about 5 mL, about 4 mL, about 3 mL, about 2 mL, about 1.9 mL, about 1.8 mL, about 1.7 mL, about 1.6 mL, about 1.5 mL, about 1.4 mL, about 1.3 mL, about 1.2 mL, about 1.1 mL, or about 1.0 mL or less of the composition.

In embodiments, the syringe comprises a composition having a shelf stability ranging from about 1 hour to about 1 week. In embodiments, the syringe comprises a composition having a shelf stability of at least about 12 hours, about 24 hours, about 36 hours, about 48 hours, or about 72 hours when stored at a temperature ranging from about −85 °C to about 25 °C. In embodiments, the syringe comprises a composition having a shelf stability of at least about 12 hours, about 24 hours, about 36 hours, about 48 hours, or about 72 hours when stored at a temperature ranging from about 15 °C to about 25 °C.

In embodiments, the storage temperature is about −80 °C. In embodiments, the storage temperature is about −20 °C. In embodiments, the storage temperature is about 4 °C. In embodiments, the storage temperature is about 21 °C. In embodiments, the kit includes lyophilized BioNVs.

In one embodiment, the kit comprises a container containing a composition comprising BioNVs of the present invention, a therapeutically effective amount of an additional therapeutic agent, such those described herein, and instructions for use.

### EXAMPLES

### Example 1: Hypoimmunogenic CAR-Based BioNV for Viral Infection

This Example pertains, in part, to the endosomal delivery of a therapeutic payload from a chimeric antigen receptor (CAR)-containing BioNV (termed mini-CAR or mini-CAR BioNV) targeting a cell that is infected with a virus.

BioNVs can be delivered to target an infected cell in one or more of the following mechanisms:
1) The payload contains peptides/proteins that can either rescue or kill a targeted cell.
2) The payload contains nucleic acids and/or endonucleases, such as gRNAs and CRISPR, respectively, that can either rescue or kill a targeted cell.
3) The payload contains a small molecules and/or nucleic acids (RNAs) that enhance or diminish cellular function of the infected cell.
4) The payload contains larger biologics, such as a virus and/or antibody, to treat virally infected cells.

Orientation and Integrity of Engineered CAR Construct: The orientation of the CAR construct will be tested using nanoparticle flow cytometry (NanoFCM), immunoblotting, and/or ELISA to confirm the orientation and topology of the surface proteins. The surface expression profile will be compared to that of the cell from which the BioNV is derived. Further Immunological Synapse (IS) quality evaluation will be performed using glass-supported planar lipid bilayer analysis coupled with a computational quantification analysis, for example as described in Cho J H, et al., “Standardized protocol for the evaluation of chimeric antigen receptor (CAR)-modified cell immunological synapse quality using the glass-supported planar lipid bilayer,” Methods Cell Biol. Vol. 173, 2023: pp. 155-171. doi: 10.1016/bs.mcb.2022.07.009. PMID: 36653082; and Gan J, et al. “Methods of Machine Learning-Based Chimeric Antigen Receptor Immunological Synapse Quality Quantification,” Methods Mol Biol; Vol. 2654, 2023: pp. 493-502. doi:10.1007/978-1-0716-3135-5_32. PMID: 37106203.

In vitro Evaluation of Binding and Delivery of Engineered mini-CAR BioNVs #1: Mini-CAR BioNVs targeting virally infected cells in vitro will be tested for delivery of a payload to the target cell. RNA labeled with a lipophilic, near-infrared fluorescent cyanine dye (e.g., DiR) will be used as the payload to evaluate the ability of the BioNV to deliver the payload into the cell. Cells expressing the CAR target will be incubated with BioNVs at varying concentrations and time points. The target cells will be assayed using one or more of fluorescence microscopy, light microscopy, and flow cytometry to visualize the ability of the BioNV payload to bind and/or penetrate the cell. BioNV administration will be compared with non-CAR BioNVs to evaluate against non-specific targeting.

In vitro Evaluation of Binding and Delivery of Engineered mini-CAR BioNVs #2: mini-CAR BioNVs will be loaded with a payload that affects one or more processes of the target cell. The BioNVs will be evaluated for their ability to bind and delivery one or more of a protein therapeutic, a gene editing modality, or a nucleic acid modality, such as an RNA therapeutic. One or more lymphocyte cell lines will be used as target cells, were the cells will be initially expanded in culture and then incubated with BioNVs at varying concentrations and time points. BioNV binding and delivery can be evaluated in one or more of the following ways:
1) For BioNVs delivering protein and/or small molecule payloads, lymphocyte phenotypes will be assayed. BioNVs will encapsulate perforin and/or nucleic acid encoding perforin to lymphocytes lacking functional perforin (e.g., from mutated PRF1 gene(s)). After incubation with BioNVs, lymphocytes will be evaluated for replenished function of perforin, by testing their ability to kill infected cells (i.e., recovery from non-functional perforin). Successful delivery of functional perforin will restore cell killing. The perforin containing mini-CAR BioNVs will be added to lymphocytes and lymphocyte target cells in co-culture in a dose dependent manner to observe target cell killing over a defined period. The concentration of perforin delivered to the lymphocyte will be analyzed via flowcytometry and/or immunoblot. These experiments will then be expanded to in vivo rescue models in mice. MCMV (Murine Cytomegalovirus) Infection Models can be used to test T-lymphocyte functionality and rescue in vivo. To test the efficacy of the approach, BioNV administration in vivo will be evaluated by one or more of fluorescent modeling (e.g., homing within the animal), biodistribution of the BioNV, lymphocyte recovery (killing recovery over a designated period), and/or viral suppression/elimination assays (e.g., PCR, RNA scope, and/or ELISA).
2) For BioNVs delivering gene editing modalities, such as CRISPR/Cas9 and gRNAs, the efficiency of gene correction or excision (post lymphocyte cytoplasmic delivery) will be tested using multiple and well-established in vitro and in vivo models, for example as described in Kennedy E M, et al. “Targeting hepatitis B virus cccDNA using CRISPR/Cas9,” Antiviral Res. Vol. 123, 2015: pp. 188-92; Kennedy E M, et al. “Optimization of a multiplex CRISPR/Cas system for use as an antiviral therapeutic,” Methods. Vol. 91, 2015: pp. 82-86; Fan M, et al. “A combinatorial CRISPR-Cas12a attack on HIV DNA,” Mol Ther Methods Clin Dev. Vol. 25, 2022: pp. 43-51; and Binda C S, et al. “CRISPR-Cas9 Dual-gRNA Attack Causes Mutation, Excision and Inversion of the HIV-1 Proviral DNA. Viruses,” Vol. 12, No. 330, 2020: pp. 1-12, each of which is hereby incorporated by reference in their entirety.

### Example 2: Hypoimmunogenic VERR-Based BioNV for Viral Infection

This Example pertains, in part, to the cytoplasmic delivery of a therapeutic payload from a viral antigen neutralizing (VAN) or viral epitope recognition receptor (VERR)-containing BioNV (termed mini-VAN or mini-VERR BioNV) targeting a cell that is infected with a virus. The mini-VAN/VERR is a BioNV contains an engineered gp120/gp41 complex (or comparable viral complexes) with reduced immunogenicity and tissue/neural toxicity while remaining highly functional, both in targeting and release mechanism activation.

BioNVs can be delivered to target an infected cell in one or more of the following mechanisms:
1) The payload contains peptides/proteins that can either rescue or kill a targeted cell.
2) The payload contains nucleic acids and/or endonucleases, such as gRNAs and CRISPR, respectively, that can either rescue or kill a targeted cell.
3) The payload contains a small molecules and/or nucleic acids (RNAs) that enhance or diminish cellular function of the infected cell.
4) The payload contains larger biologics, such as a virus and/or antibody, to treat virally infected cells.

Orientation and Integrity of Engineered gp120/gp41 Complexes: The orientation of the gp120/gp41 complexes will be tested using one or more of nanoparticle flow cytometry (NanoFCM), immunoblotting, and ELISA to confirm the topology of the complex compared to those within the cell from which the BioNVs are derived.

Targeting Specificity, Precision, Affinity and Avidity Studies for the Engineered gp120/gp41 Complex: Cells expressing target receptors, CD4 and chemokine receptors CCR5 and CXCR4, will be used to evaluate the BioNV targeting. To ensure the complex readily targets its receptor on target cells, targeting specificity, precision affinity and avidity studies will be carried out between the complex and one or more fluorescent reporter tagged receptors. The precision and avidity readouts will be analyzed by FRET and/or flow cytometry under steady and live state environments, respectively. The specificity and affinity of the complex will be measured using the CCR5 and CXCR4 chemokine signaling pathway properties. When these receptors are engaged by the complex, an influx of calcium into the cell and tyrosine phosphorylation of tyrosine kinase, Pyk2, will occur. The degree of calcium flux in real time can be measured by changes in calcium-sensitive fluorescence dyes, such as Fura-2, Fluo-4, and/or Oregon Green. The phosphorylation of Pyk2 will be assayed by ELISA and/or immunoblotting with phosphor-specific antibodies. Each analysis will be compared with a wild-type gp120/gp41 complex standard controls (expressed, for example, from LAI and/or NL4-3 cell lines for CXCR4, and JR-CSF, 49.5, and/or KP1 cell lines for CCR5).

In vitro Evaluation of Binding and Delivery of Engineered mini-VAN/VERR BioNVs #1: Mini-VAN/VERR BioNVs targeting T-lymphocytes in vitro will be tested for delivery of a payload to the target cell. RNA labeled with a lipophilic, near-infrared fluorescent cyanine dye (e.g., DiR) will be used as the payload to evaluate the ability of the BioNV to deliver the payload into the cell. Cells expressing the VAN/VERR target will be incubated with BioNVs at varying concentrations and time points. The target cells will be assayed using one or more of fluorescence microscopy, light microscopy, and flow cytometry to visualize the ability of the BioNV payload to bind and/or penetrate the cell. BioNV administration will be compared with a wild-type gp120/gp41 complex (expressed from LAI and/or NL4-3 cell lines for CXCR4, and JR-CSF, 49.5, and/or KP1 cell lines for CCR5) and non-targeted BioNVs.

In vitro Evaluation of Binding and Delivery of Engineered mini-VAN/VERR BioNVs #2: Mini-VAN/VERR BioNVs will be loaded with a payload that affects one or more processes of the target cell. The BioNVs will be evaluated for their ability to bind and delivery one or more of a protein therapeutic, a gene editing modality, or a nucleic acid modality, such as an RNA therapeutic. The target cells will be lymphocytes initially, expanded to other cell types upon gp120 modification. BioNV binding and delivery can be evaluated in one or more of the following ways:
1) For BioNVs delivering protein payloads, lymphocyte phenotypes will be assayed. BioNVs will encapsulate perforin and/or nucleic acid encoding perforin to lymphocytes lacking functional perforin (e.g., from mutated PRF1 gene(s)). After incubation with BioNVs, lymphocytes will be evaluated for replenished function of perforin, by testing their ability to kill infected cells (i.e., recovery from non-functional perforin). Successful delivery of functional perforin will restore cell killing. The perforin containing mini-VAN/VERR BioNVs will be added to lymphocytes and lymphocyte target cells in co-culture in a dose dependent manner to observe target cell killing over a defined period. The concentration of perforin delivered to the lymphocyte will be analyzed via flowcytometry and/or immunoblot. These experiments will then be expanded to in vivo rescue models in mice. MCMV (Murine Cytomegalovirus) Infection Models can be used to test T-lymphocyte functionality and rescue in vivo. To test the efficacy of the approach, BioNV administration in vivo will be evaluated by one or more of fluorescent modeling (e.g., homing within the animal), biodistribution of the BioNV, lymphocyte recovery (killing recovery over a designated period), and/or viral suppression/elimination assays (e.g., PCR, RNA scope, and/or ELISA).
2) For BioNVs delivering gene editing modalities, such as CRISPR/Cas9 and gRNAs, the efficiency of gene correction or excision (post lymphocyte cytoplasmic delivery) will be tested using multiple and well-established in vitro and in vivo models, for example as described in Kennedy E M, et al. “Targeting hepatitis B virus cccDNA using CRISPR/Cas9,” Antiviral Res. Vol. 123, 2015: pp. 188-92; Kennedy E M, et al. “Optimization of a multiplex CRISPR/Cas system for use as an antiviral therapeutic,” Methods. Vol. 91, 2015: pp. 82-86; Fan M, et al. “A combinatorial CRISPR-Cas12a attack on HIV DNA,” Mol Ther Methods Clin Dev. Vol. 25, 2022: pp. 43-51; and Binda C S, et al. “CRISPR-Cas9 Dual-gRNA Attack Causes Mutation, Excision and Inversion of the HIV-1 Proviral DNA. Viruses,” Vol. 12, No. 330, 2020: pp. 1-12, each of which is hereby incorporated by reference in their entirety.

### Example 3: Generating BioNVs Via Serial Extrusion

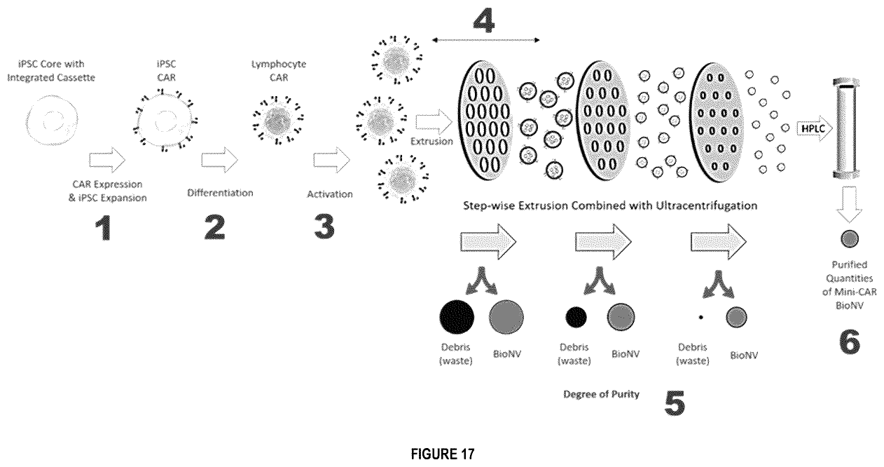

Biomimetic Nanovesicles (BioNVs) can be produced from hypoimmunogenic cell lines as illustrated in the scheme depicted in FIG. 17. The following protocols outline experiments for mini-CAR and/or mini-VAN BioNVs. The mini-CAR and/or mini-VAN BioNVs can be derived from any cell type. The following outlines use BioNVs derived from either activated or non-activated Natural Killer lymphocytes (NK cells). The BioNVs can be designed to target any antigen receptor, as described herein.

The level of CAR expression can be measured in the hypoimmunogenic cell line using a combination of flow cytometry and iodixanol density gradient (e.g., STEP 1 of FIG. 17).

The differentiation of the iPSC-expressing surface CAR, VERR, and/or viral ligand into lymphocytes can be analyzed by lymphocyte marker identification including, for example CD4/CD8 (T-cells) or CD56/CD16 (Natural Killers cells), among other cell surface markers (e.g., STEP 2 of FIG. 17). The expression profile can be determined via flow cytometry, RT-PCR, and/or CRISPR-based analytics.

Next, the activation of the lymphocytes can be achieved using biomarker antigen-coated beads in low, pre-determined concentrations over the course of two weeks in two stages (e.g., STEP 3 of FIG. 17). This process can also analyze the quality of the Immunological Synapse (IS) between the CAR, VERR, and/or viral ligand and the antigen-coated beads, using well-established protocols to measure i) the quantification of F-Actin accumulation at the site of synapse formation, ii) the distribution of pZeta at synapse, iii) the clustering of an antigen through the IS location, and/or iv) the polarization of lytic granules (LGs) that contain perforin and granzymes.

After the activation of the lymphocytes, the cells are expanded using established protocols (e.g., STEP 4 of FIG. 17). After expansion, the levels of perforin and granzyme (or other lumen payloads if applicable) are analyzed per cell population to ensure consistent concentration levels on a per-batch basis. This is accomplished using a series of qPCR, immunoblotting, flow cytometry, and/or mass spectrometry. The expansion step may not be necessary if a large enough cell population from Step 3 can be achieved.

Once the cells are activated to produce the desired therapeutic protein(s), they are expanded, harvested, washed several times, and then placed into a buffered extrusion medium. The cells are then wholly processed via serial extrusion through each step of the polycarbonate filter system that consists of diminishing pore size (e.g., STEP 5 of FIG. 17). In the initial extrusion step of the serial extrusion process, the nucleus (along with nuclear components including nuclear pores, genomic material, and transcription factors) and mitochondria are eliminated. The sample is then treated with endonuclease, e.g., BENZONASE. BENZONASE is a non-specific, recombinant endonuclease that cleaves all types of DNA and RNA variants into non-functional fragments <8 soluble base pairs. This leads to the highest reduction of nucleic acid load on a per sample and scalable basis and does not interfere with BioNV membrane chemistry. The cleavage process also eliminates nucleic viscosity, allowing for subsequent loading and passage of materials through the next set of extrusion filters.

The serial extrusion process will avoid the elimination of other organelles such as the Golgi Apparatus or the ER. The membrane system of these organelles is highly evolved to traffic vesicles (release and uptake) between folded membranes. For example, the cis and trans face of the Golgi Apparatus contain unique lipid compositions that facilitate low energy barrier absorption and release in the trafficking of vesicles. These components are relatively low in the cytoplasmic membrane. Therefore, isolating the cytoplasmic membrane for BioNV derivation is not as favorable. As the BioNVs are passed through the polycarbonate filters in the serial extrusion process, they undergo destruction and spontaneous formation based on the pore size. This process results in BioNVs containing membranes with a homogenous mixture of cytoplasm, Golgi, and ER lipid content and protein components that can considerably increase their affinity for cellular and tissue delivery uptake in comparison to BioNVs processed to eliminate these organelles. These features could translate to better and more consistent uptake of BioNVs into targeted cells at much lower doses than systems that do not incorporate these properties.

After the extrusion step, the BioNVs are passed through an α-CD3 HPLC (FPLC in scale-up) column to remove the low percent (approximately 0.05%) of inverted BioNVs that spontaneously form during the serial extrusion process (e.g., STEP 6 of FIG. 17). This is done to ensure the resultant BioNVs have homogenous directionality with respect to the membranes. Low loss of yield occurs during this step, as it is a flow-through process to capture impurities. Once the BioNVs have been collected after the HPLC/FPLC step, they are tested through a standardization process.

The standardization process includes one or more the following assays:
BioNV homogeneity: the use of Nanoparticle Flow Cytometry (NanoFCM) can confirm BioNV concentration, homogeneity of size, the density of the BioNVs, and/or the homogeneity of the BioNV lumen constituents.
Concentration of lumen payload: NanoFM technology can be used to determine the type and concentration of the nucleic acids/proteins that are packaged into the lumen of the BioNVs. These data can be confirmed in parallel with one or more methods including immunoblot, mass spectrometry, and BCA analyses to determine the nucleic acid and protein content of BioNVs.
BioNV Stability: a combination of nanoparticle tracking analysis (NTA), dynamic light scattering (DLS), and electron microscopy (EM) can be used in combination with immunoblot and mass spectrometry analyses to determine the physical and biochemical features of the BioNVs over 8 to 10 months. Data from these assays can include the protein expression profile, the degree of intact BioNV membranes/packaging, and/or the degree of aggregation.
Membrane Integrity: the integrity of the BioNV membranes is evaluated using calcein release assays combined with NanoFCM to assess membrane permeability. The results can provide insight into the leakage properties of the BioNVs against standardized BioNV panels.
Quality of lumen payload: the quality of the lumen-packaged payloads can be determined using multiple analytic assays, depending on the nature of the payload. In instances where the deliverable is a nucleic acid, qPCR and/or sequencing over 8 to 10 months can be used to check the integrity and quantity of the nucleic acid payloads. For proteins, an analysis of the BioNV constituents using one or more of NanoFCM, mass spectrometry, and immunoblot analyses can be used to analyze the protein payload.
CAR Quality and Surface Density: CAR, VERR, and/or viral ligand surface density can be determined using NanoFCM, mass spectrometry, and/or immunoblot analyses. CAR, VERR, and/or viral ligand surface density is expected to be at least about 5-fold to at least about 10-fold higher in BioNVs compared to whole cell surface densities. This could considerably enhance targeting to the antigen in comparison to a whole cell. CAR, VERR, and/or viral ligand quality can be determined at the cellular stage, as described above (e.g., as in STEP 3). A mathematical model can be used to extrapolate cellular quality data and apply it to the BioNVs in relation to efficacy study data outcomes.
BioNV Functionality: BioNVs can be tested for basic functionality, including multiple and defined standardization assays, such as in vitro cellular uptake into targeted cells with and without expressed antigen, as well their ability to cross dense tissues such as those in human retinal models. Following these basic functionality assays, which can be performed immediately after the serial extrusion process, pre-clinical studies will address the remainder of the quality and functionality properties of the BioNVs.

Following the extrusion process, the mini-CAR BioNVs (as well as VERR and/or viral ligand BioNVs) will be segmented based on activation immunological synapse (IS) binding into the following groups:
1) Low quality IS pool of whole cells (activated), where “low quality” refers to degree of IS binding;
2) Moderate quality IS pool of whole cells (activated), where “moderate quality” refers to degree of IS binding relative to “low” and “high” qualities;
3) High quality IS pool of whole cells (activated), where “high quality” refers to degree of IS binding; and
4) High quality IS pool of whole cells (non-activated).

BioNVs can be cryopreserved at this stage.

Statistical analysis will be used to evaluate and address batch-to-batch consistency. Upper and lower control limits will be based upon data using controls (e.g., means, 95% confidence intervals, range, and/or percentile distribution).

### DEFINITIONS

The following definitions are used in connection with the invention disclosed herein. Unless defined otherwise, all technical and scientific terms used herein have the same meaning as commonly understood to one of skill in the art to which this invention belongs.

An “effective amount,” or “therapeutically effective amount,” is an amount that is effective for treating, preventing, or ameliorating a disease or disorder such as those described herein, or an amount that is intended to reduce the number of virally-infected cells, including latently infected cells, and/or reduce the amount of detectable viral DNA (or cDNA), RNA, and/or protein in a subject.

As used herein, “a,” “an,” or “the” can mean one or more than one.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present invention, or embodiments thereof, and may alternatively be described using alternative terms such as “consisting of” or “consisting essentially of.”

In embodiments, “BioNVs,” as referred to herein, are allogeneic, hypoimmunogenic biomimetic nanovesicles (NVs) which comprise at least one surface-oriented, membrane-embedded CAR, VERR, and/or viral ligand. In embodiments, “nanovesicles (NVs),” as referred to herein, are lipid-bound vesicles on the order of about 10 nm to about 1200 nm in size which encapsulate an aqueous core. In embodiments, lipid-bound NVs can form using lipid monolayers, lipid bilayers, or maintain multilamellar forms. In embodiments, BioNV refers to biologically-derived nano-sized vesicles that can have designed biological functionalization. In embodiments, BioNVs are “biomimetic” in that they are derived from endogenous cellular material; more specifically, they substantially recapitulate plasma membrane material found in cells. In embodiments, the cells from which BioNVs originate can include stem cells of any kind, including cell types differentiated from said stem cells. In embodiments, BioNVs are substantially free of encapsulated cellular debris including nucleic acid, organelles, or organelle parts. In embodiments, BioNVs are characterized as having one or more, 2 or more, 3 or more, 4 or more, 5 or more, or 6 or more of the following:
a. being about 10 nm to about 1200 nm in size;
b. having a total volume of about 500 nm³ to about 5 μm³ (assuming spherical shape);
c. having a content of at least one phospholipid and cholesterol;
d. having a surface membrane having one or more of CD34, CCL21, PD-L1 (in BioNVs derived from non-activated cell sources), FasL, SerpinB9, H2-M3, CD47, CTLA-4, CD24, CD200, MFG-E8, NCAM, and/or α-phagocytic integrin, or a chimera of any one or more thereof; having a surface membrane substantially lacking T cell receptor components (TRAC and/or TRBC), MHC class I components, and/or MHC class II components, lacking proteins of one or more of HLA-A, HLA-B, HLA-C, HLA-E or HLA-G (but not both of HLA-E and HLA-G), HLA-F, and/or CIITA, SerpinB9, substantially lacking proteins inside the vesicle of one or more of IL-4, IL-6, IL-10, and/or IL-16;
e. having a membrane-embedded CAR and/or VERR and/or viral ligand comprising a target-binding moiety which can include an antibody or antibody format selected from one or more of a monoclonal antibody, polyclonal antibody, antibody fragment, Fab, Fab′, Fab′-SH, F(ab′)2, Fv, single chain Fv (scFv), diabody, nanobody, linear antibody, bispecific antibody, multi-specific antibody, chimeric antibody, humanized antibody, human antibody, fusion protein comprising the antigen-binding portion of an antibody, Bispecific T cell Engagers (BITE), or by a variable heavy chain IgG fragment VHH or VNAR or through a T-Cell Receptor (TCR);
f. being capable of adsorbing and/or encapsulating a payload content of one or more perforins, one or more granzymes, one or more cytokines, one or more cytotoxic proteins, one or more checkpoint inhibiting agents, gene editing payloads, antibodies or antibody fragments, small molecule inhibitors, biologics, radionuclides, tracing agents, dyes, fluorescent proteins, among other therapeutic payloads, and/or any combination thereof; and
g. being capable of not causing a deleterious immune reaction in subjects.

In embodiments, “induced pluripotent stem cells,” “iPSCs,” or “reprogrammed induced pluripotent stem cells,” are stem cells that originate from differentiated cells and are reprogrammed back into an embryonic-like pluripotent state. iPSCs can generally propagate indefinitely and become any cell type of the organism they originate.

In embodiments, “allogeneic,” as used herein, refers to biological material, tissues, or cells, which are genetically dissimilar and originally immunological incompatible, despite originating from the same species. Allogeneic BioNVs, for example, are material that originates from a first subject (iPSC donor) and can be provided to any number of distinct subjects who are not genetically identical.

In embodiments, “hypoimmunogenic” or “hypoimmune,” as used herein in reference to a modified cell and/or BioNV, refers to a reduced capacity to generate an immunological response. In embodiments, iPSCs and BioNVs can be hypoimmunogenic due to reduced or ablated expression and/or activity of one or more immunogenic cell surface proteins and/or secreted proteins, such as TCR proteins, CRS proteins, MHC class I or MHC class II proteins, etc. In embodiments, iPSCs and BioNVs can be hypoimmunogenic due to expression of one or more immunoprotective cell surface proteins, such as CD47, CD34, CD24, CD200, α-phagocytic integrins, etc. In embodiments, BioNVs can be hypoimmunogenic due to not triggering CRS in a subject and/or not inducing HLA incompatibility.

In embodiments, “knocking-out,” “silencing,” “inactivating,” “disrupting,” or “blocking,” and their equivalencies, with respect to transcription, gene, or protein expression, refers to an amount of transcription, gene or protein expression which is reduced from a normal state or less than the wild-type state in a particular cell subset. The reduction can be significant so that no gene expression occurs, or a negligible amount of protein expression occurs.

In embodiments, “overexpression,” as used herein, refers to an amount of transcription, gene or protein expression which is increased from a normal state or more than the wild-type state in a particular cell subset.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

As used herein, all headings are simply for organization and are not intended to limit the disclosure in any manner. The content of any individual section may be equally applicable to all sections.

### INCORPORATION BY REFERENCE

All patents and publications referenced herein are hereby incorporated by reference in their entireties, including published PCT application, WO 2020/227369, filed May 6, 2020, titled “Tailored Hypoimmune Nanovesicle Delivery Systems for Cancer Tumors,” and published U.S. non-provisional application, US20220040106 A1, filed Aug. 3, 2021, titled “Tailored Hypoimmune Nano vesicular Delivery Systems for Cancer Tumors, Hereditary and Infectious Diseases.”

What is claimed is:

1. A method of treating or preventing a viral infection, comprising administering to a subject in need thereof a therapeutically effective amount of:
(a) a biomimetic nanovesicle (BioNV), the BioNV being targeted to one or more markers of cellular infection with the virus; and
(b) one or more checkpoint inhibiting agents.

2. A method of treating or preventing a viral infection, comprising administering to a subject in need thereof a therapeutically effective amount of a biomimetic nanovesicle (BioNV), the BioNV being targeted to one or more markers of cellular infection with the virus, wherein the subject is undergoing treatment with one or more checkpoint inhibiting agents.

3. A method of treating or preventing a viral infection, comprising administering to a subject in need thereof a therapeutically effective amount of one or more checkpoint inhibiting agents, wherein the subject is undergoing treatment with a biomimetic nanovesicle (BioNV), the BioNV being targeted to one or more markers of cellular infection with the virus.

4. A method of treating or preventing a viral infection, comprising administering to a subject in need thereof a therapeutically effective amount of a biomimetic nanovesicle (BioNV), the BioNV:
(i) being targeted to one or more markers of cellular infection with the virus; and
(ii) comprising one or more checkpoint inhibiting agents.

5. The method of any one of claims 1-4, wherein the marker is expressed on one or more of T cells, dendritic cells, or macrophages, or any cell type in an HIV-infected cell reservoir.

6. The method of claim 5, wherein the T cells are CD4+ T cells or CD8+ cytotoxic T cells (CTL).

7. The method of any one of claims 1-6, wherein the marker is or comprises a sialic acid-binding immunoglobulin-like lectin (Siglec) molecule.

8. The method of claim 7, wherein the marker is or comprises Siglec-1.

9. The method of any one of claims 1-6, wherein the marker is one or more of CD2, CD3, CD4, CXCR4, CCR5, CD20, CD25, CD30, CD32a, CD69, CD91, CD160, CD257, LAG-3, CD147, CD231, CEACAM1, PLXNB2, HLA-DR, PD-1, CTLA-4, TIGIT, LAG-3, TIM-3, BTK, Cyclophilin B, Sec62, Rab10, SPCS, or a combination thereof.

10. The method of any one of preceding claims, wherein the BioNV is derived from a modified cell, and wherein the modified cell is a stem cell, an induced pluripotent stem cell (iPSC), a reprogrammed pluripotent or multipotent cell, an embryonic stem cell, a mesenchymal stem cell, or a differentiated cell which originates from any modified cell thereof.

11. The method of claim 10, wherein the modified cell is an iPSC.

12. The method of claim 10, wherein the modified cell is a T cell, helper T cell, T-memory cell, or NK cell.

13. The method of any one of claims 10-12, wherein the modified cell substantially lacks one or more MHC class I proteins, MHC class II proteins, T cell receptor (TCR) proteins, and/or cytokine release syndrome (CRS) proteins.

14. The method of any one of claims 10-13, wherein the modified cell has reduced or ablated expression of a β2-macroglobulin (B2M) gene and/or reduced or ablated MHC class I protein expression and/or activity.

15. The method of any one of claims 10-13, wherein the modified cell has reduced or ablated expression of a MHC II transactivator (CIITA) gene and/or reduced or ablated MHC class II protein expression and/or activity.

16. The method of any one of claims 10-15, wherein the modified cell has reduced or ablated expression of an HLA-A gene and/or reduced or ablated HLA-A protein expression and/or activity.

17. The method of any one of claims 10-16, wherein the modified cell has reduced or ablated expression of an HLA-B gene and/or reduced or ablated HLA-B protein expression and/or activity.

18. The method of any one of claims 10-17, wherein the modified cell has reduced or ablated expression of an HLA-C gene and/or reduced or ablated HLA-C protein expression and/or activity.

19. The method of any one of claims 10-18, wherein the modified cell has reduced or ablated expression of an HLA-E or HLA-G gene and/or reduced or ablated HLA-E or HLA-G protein expression and/or activity.

20. The method of any one of claims 10-19, wherein the modified cell has reduced or ablated expression of an HLA-F gene and/or reduced or ablated HLA-F protein expression and/or activity.

21. The method of any one of claims 10-20, wherein the modified cell has reduced or ablated expression of a T cell alpha constant (TRAC) gene and/or reduced or ablated TRAC protein expression and/or activity.

22. The method of any one of claims 10-21, wherein the modified cell has reduced or ablated expression of a T cell beta constant (TRBC) gene and/or reduced or ablated TRBC protein expression and/or activity.

23. The method of any one of claims 10-22, wherein the modified cell has reduced or ablated expression of a PD-1 gene and/or reduced or ablated PD-1 protein expression and/or activity, wherein the modified cell is activated; or the modified cell has expression or increased expression of a PD-1 gene and/or gene product, wherein the modified cell is not activated.

24. The method of any one of claims 10-23, wherein the modified cell has reduced or ablated expression of an IL-4 gene and/or reduced or ablated IL-4 protein expression and/or activity.

25. The method of any one of claims 10-24, wherein the modified cell has reduced or ablated expression of an IL-6 gene and/or reduced or ablated IL-6 protein expression and/or activity.

26. The method of any one of claims 10-25, wherein the modified cell has reduced or ablated expression of an IL-10 gene and/or reduced or ablated IL-10 protein expression and/or activity.

27. The method of any one of claims 10-26, wherein the modified cell has reduced or ablated expression of an IL-16 gene and/or reduced or ablated IL-16 protein expression and/or activity.

28. The method of any one of claims 10-27, wherein the modified cell has reduced or ablated expression of a SerpinB9 gene and/or reduced or ablated SerpinB9 protein expression and/or activity.

29. The method of any one of claims 10-27, wherein the modified cell expresses or has increased expression of a SerpinB9 gene and/or gene product.

30. The method of any one of claims 10-29, wherein the modified cell expresses or has increased expression of a CD34 gene and/or gene product.

31. The method of any one of claims 10-30, wherein the modified cell expresses or has increased expression of a CCL2 gene and/or gene product.

32. The method of any one of claims 10-31, wherein the modified cell expresses or has increased expression of a PD-L1 gene and/or gene product, and wherein the modified cell is not activated;
or wherein the modified cell has reduced or ablated expression of a PD-L1 gene and/or gene product, and wherein the modified cell is activated.

33. The method of any one of claims 10-32, wherein the modified cell expresses or has increased expression of a H2-M3 gene and/or gene product.

34. The method of any one of claims 10-33, wherein the modified cell expresses or has increased expression of a CD47 gene and/or gene product.

35. The method of any one of claims 10-34, wherein the modified cell expresses or has increased expression of a CD24 gene and/or gene product.

36. The method of any one of claims 10-35, wherein the modified cell expresses or has increased expression of a chimeric CD24/CD47 gene and/or gene product.

37. The method of any one of claims 10-36, wherein the modified cell expresses or has increased expression of a chimeric CD200 gene and/or gene product.

38. The method of any one of claims 10-37, wherein the modified cell expresses or has increased expression of a chimeric CD24/CD200 gene and/or gene product or a chimeric CD47/CD200 gene and/or gene product.

39. The method of any one of claims 10-38, wherein the modified cell expresses or has increased expression of a chimeric CTLA-4 gene and/or gene product.

40. The method of any one of claims 10-39, wherein the modified cell expresses or has increased expression of a chimeric MFG-E8 gene and/or gene product.

41. The method of any one of claims 10-40, wherein the modified cell expresses or has increased expression of an NCAM gene and/or gene product.

42. The method of any one of claims 10-41, wherein the modified cell expresses or has increased expression of a chimeric α-phagocytic integrin gene and/or gene product.

43. The method of any one of claims 10-42, wherein the modified cell expresses or has increased expression of a FasL gene and/or gene product.

44. The method of any one of claims 10-43, wherein the modified cell wherein does not overexpress a FasL gene and/or gene product.

45. The method of any one of claims 10-44, wherein the modified cell has reduced or ablated expression and/or activity of 3 or more immunogenic proteins, 4 or more immunogenic proteins, 5 or more immunogenic proteins, 6 or more immunogenic proteins, 7 or more immunogenic proteins, 8 or more immunogenic proteins, 9 or more immunogenic proteins, 10 or more immunogenic proteins, 11 or more immunogenic proteins, or 12 or more immunogenic proteins.

46. The method of any one of claims 10-45, wherein the modified cell expresses or has increased expression of 3 or more immunoprotective proteins, 4 or more immunoprotective proteins, 5 or more immunoprotective proteins, 6 or more immunoprotective proteins, 7 or more immunoprotective proteins, 8 or more immunoprotective proteins, 9 or more immunoprotective proteins, or 10 or more immunoprotective proteins.

47. The method of any one of claims 10-46, wherein the modified cell is allogeneic.

48. The method of any one of claims 10-47, wherein the modified cell does not cause an immune reaction in patients to which it or a BioNV derived therefrom is administered.

49. The method of any one of the preceding claims, wherein the BioNV comprises one or more targeting agents directed to the marker on its surface.

50. The method of claim 49, wherein the one or more targeting agents comprises a chimeric antigen receptor (CAR), viral epitope recognition receptor (VERR), and/or viral ligand.

51. The method of claim 50, wherein the targeting agent is a ligand for a receptor or a receptor for a ligand, optionally wherein the VERR and/or viral ligand is a gp120/gp41 complex.

52. The method of claim 50, wherein the targeting agent comprises one or more of a transmembrane domain, an intracellular domain, a costimulatory domain, and/or a signaling domain; or wherein the targeting agent lacks an intracellular portion.

53. The method of claim 52, wherein the transmembrane domain is derived from CD28, CD3ζ, CD4, CD8α, or ICOS, or a fragment thereof.

54. The method of claim 52, wherein the intracellular domain comprises an intracellular signaling domain of a CD3ζ-chain and/or one or more co-stimulatory molecules, optionally selected from CD28, 4-1BB, ICOS, CD27, and OX40.

55. The method of any one of claims 49-54, wherein the targeting agent is an antibody or an antibody format.

56. The method of claim 55, wherein the antibody or antibody format is selected from one or more of a CAR, VERR, viral ligand, Fab, Fab′, Fab′-SH, F(ab′)2, Fv, single chain Fv (scFv), diabody, nanobody, monoclonal antibody, polyclonal antibody, antibody fragment, linear antibody, bispecific antibody, multi-specific antibody, chimeric antibody, humanized antibody, human antibody, and fusion protein comprising the antigen-binding portion of an antibody.

57. The method of claim 56, wherein the antibody format is a CAR scFv and/or a VERR-scFv fusion.

58. The method of any one of claims 1-57, wherein the BioNV is about 10 nm to about 1200 nm in size.

59. The method of any one of claims 1-58, wherein the BioNV is about 10 nm to about 100 nm in size.

60. The method of any one of claims 1-58, wherein the BioNV is about 100 nm to about 200 nm in size.

61. The method of any one of claims 1-58, wherein the BioNV is about 200 nm to about 500 nm in size.

62. The method of any one of claims 1-58, wherein the BioNV is about 500 nm to about 1200 in size.

63. The method of any one of the preceding claims, wherein the BioNV substantially lacks expression and/or activity of HLA-A, HLA-B, HLA-C, HLA-F, CIITA, IL-6, TRAC, TRBC, and one of either HLA-E or HLA-G.

64. The method of any one of the preceding claims, wherein the BioNV substantially lacks expression and/or activity of HLA-A, HLA-B, HLA-C, HLA-F, CIITA, IL-6, TRAC, TRBC, SerpinB9, and one of either HLA-E or HLA-G.

65. The method of any one of the preceding claims, wherein the BioNV substantially lacks expression and/or activity of HLA-A, HLA-B, HLA-C, HLA-F, CIITA, IL-6, TRAC, TRBC, SerpinB9, CD200, one of either HLA-E or HLA-G, and one or more of IL-4, IL-10, and IL-16.

66. The method of any one of the preceding claims, wherein the BioNV comprises a membrane-embedded α-phagocytic integrin, CCL2, H2-M3, FasL, MFG-E8, and PD-L1 and/or CTLA-4; and
one of either CD24, CD47, CD200, chimeric CD24/CD47, chimeric CD24/CD200, and chimeric CD47/CD200, or two of either CD24, CD47, and CD200.

67. The method of any one of claims 1-65, wherein the BioNV comprises a membrane-embedded α-phagocytic integrin, CCL2, H2-M3, FasL, MFG-E8, and PD-L1 and/or CTLA-4; and
one of either CD24, CD47, CD200, chimeric CD24/CD47, chimeric CD24/CD200, and chimeric CD47/CD200, or two of either CD24, CD47, and CD200.

68. The method of any one of claims 1-65, wherein the BioNV comprises a membrane-embedded CD200 protein and substantially lacks either CD24 or CD47.

69. The method of any one of claims 1-65, wherein the BioNV substantially lacks expression and/or activity of SerpinB9 and CD200.

70. The method of any one of the preceding claims, wherein the checkpoint inhibiting agent is an agent that modulates one or more of PD-1, PD-L1, PD-L2, CTLA-4, TIM-3, LAG-3, B7-H3, B7-H4, A2aR, CD73, NKG2A, PVRIG/PVRL2, CEACAM1, CEACAM 5/6, FAK, CCL2/CCR2, LIF, CD47/SIRPα, CSF-1 (M-CSF)/CSF-1R, IL-1, IL-1R3, IL-8, SEMA4D, Ang-2, CLEVER-1, Axl, Phosphatidylserine.

71. The method of any one of the preceding claims, wherein the checkpoint inhibiting agent is an antibody or an antibody format.

72. The method of claim 71, wherein the antibody or antibody format is specific for one of PD-1, PD-L1, PD-L2, CTLA-4, TIM-3, LAG-3, B7-H3, B7-H4, A2aR, CD73, NKG2A, PVRIG/PVRL2, CEACAM1, CEACAM 5/6, FAK, CCL2/CCR2, LIF, CD47/SIRPα, CSF-1 (M-CSF)/CSF-1R, IL-1, IL-1R3, IL-8, SEMA4D, Ang-2, CLEVER-1, Axl, Phosphatidylserine.

73. The method of claim 71 or 72, wherein the antibody or antibody format is selected from one or more of a CAR, VERR, VNAR, VHH, viral ligand, Fab, Fab′, Fab′-SH, F(ab′)2, Fv, scFv, diabody, nanobody, monoclonal antibody, polyclonal antibody, antibody fragment, linear antibody, bispecific antibody, multi-specific antibody, chimeric antibody, humanized antibody, human antibody, and fusion protein comprising the antigen-binding portion of an antibody.

74. The method of any one of claims 71-73, wherein the antibody or antibody format is specific for PD-1 and is selected from nivolumab, pembrolizumab, and pidilizumab.

75. The method of any one of claims 71-74, wherein the antibody or antibody format is specific for PD-L1 and is selected from atezolizumab, avelumab, durvalumab, and BMS-936559.

76. The method of any one of claims 71-75, wherein the antibody or antibody format is specific for CTLA-4 and is selected from ipilimumab, tremelimumab, AGEN1884, and RG2077.

77. The method of any one of the preceding claims, wherein the viral infection is a latent infection.

78. The method of any one of the preceding claims, wherein the viral infection is a retroviral infection.

79. The method of claim 78, wherein the retroviral infection is a human immunodeficiency virus (HIV) infection.

80. The method of claim 79, wherein the HIV is HIV-1 or HIV-2.

81. The method of claim 80, wherein the HIV-1 is one of Group M, Group N, Group O, and Group P.

82. The method of claim 80 or 81, wherein the HIV-1 is Group M and a subtype selected from subtype A, B, C, D, F, G, H, I, J, K, and L.

83. The method of claim 78, wherein the retroviral infection is one of bovine immunodeficiency virus, caprine arthritis encephalitis virus, equine infectious anemia virus, feline immunodeficiency virus, Jembrana disease virus, puma lentivirus, simian immunodeficiency virus, visna-maedi virus, and human T-lymphotropic virus type 1 (HTLV-1) and HTLV-2.

84. The method of any one of the preceding claims, wherein the BioNV comprises one or more perforin molecules.

85. The method of any one of the preceding claims, wherein the BioNV comprises one or more granzyme molecules.

86. The method of claim 85, wherein the granzyme molecules are selected from granzyme A, B, H, K, and M.

87. The method of any one of the preceding claims, wherein the BioNV comprises one or more perforin and one or more granzyme molecules derived from a cell from which the BioNV is derived.

88. The method of any one of claims 1-86, wherein the BioNV comprises one or more perforin and one or more granzyme molecules exogenously added to the BioNV.

89. The method of any one of the preceding claims, further comprising administering one or more antiretroviral therapies.

90. The method of any one of claims 1-88, wherein the BioNV encapsulates one or more antiretroviral therapies.

91. The method of claim 89 or 90, wherein the antiretroviral therapy is a non-nucleoside reverse transcriptase inhibitor (NNRTI), optionally selected from efavirenz (SUSTIVA), rilpivirine (EDURANT) and doravirine (PIFELTRO).

92. The method of claim 89 or 90, wherein the antiretroviral therapy is a nucleoside or nucleotide reverse transcriptase inhibitor (NRTI), optionally selected from abacavir (ZIAGEN), tenofovir (VIREAD), emtricitabine (EMTRIVA), lamivudine (EPIVIR), zidovudine (RETROVIR), emtricitabine/tenofovir (TRUVADA), and emtricitabine/tenofovir alafenamide (DESCOVY).

93. The method of claim 89 or 90, wherein the antiretroviral therapy is a protease inhibitor (PI), optionally selected from atazanavir (REYATAZ), darunavir (PREZISTA), and lopinavir/ritonavir (KALETRA).

94. The method of claim 89 or 90, wherein the antiretroviral therapy is an integrase inhibitor, optionally selected from bictegravir sodium/emtricitabine/tenofovir alafenamide fumar (BIKTARVY), raltegravir (ISENTRESS), and dolutegravir (TIVICAY).

95. The method of claim 89 or 90, wherein the antiretroviral therapy is an entry or fusion inhibitor, optionally selected from enfuvirtide (FUZEON) and maraviroc (SELZENTRY).

96. The method of any one of claims 89-95, wherein the antiretroviral therapy comprises at least two, or at least three, or at least four of the antiretroviral therapies in combination.

97. The method of any one of the preceding claims, wherein the BioNV comprises one or more gene editing payloads and one or more antiretroviral therapies.

98. The method of any one of the preceding claims, further comprising co-administering a whole cell therapy.

99. The method of any one of the preceding claims, further comprising administering an additional therapeutic agent.

100. The method of any one of the preceding claims, wherein the BioNV is stored at about −80 °C or suitable for storage at about −80 °C.

101. The method of any one of the preceding claims, wherein the BioNV is lyophilized.

102. The method of any one of the preceding claims, wherein the method causes the excision of viral nucleic acid from one or more infected cells, optionally HIV-infected cells.

103. The method of any one of claims 1-101, wherein the method causes the excision of viral nucleic acid from one or more latently infected cells, optionally latently HIV-infected cells.

104. The method of any one of claims 1-101, wherein the method causes a prevention of clonal expansion of infected cells, optionally HIV-infected cells.

105. The method of any one of claims 1-101, wherein the method causes eradication, stoppage, halt or end of HIV or infection symptoms, or the progression of the symptoms or virus in the subject.

106. The method of any one of claims 1-101, wherein the method induces and maintains sustained viral control, optionally selected from undetectable levels of plasma viremia and/or maintenance of less than about 50 copies/mL of HIV viral RNA, optionally as assayed by a polymerase chain reaction (PCR) test, a branched chain DNA (bDNA) test or a nucleic acid sequence-based amplification (NASBA) test.

107. A composition for the treatment of a viral infection comprising:
an allogeneic, hypoimmunogenic biomimetic nanovesicle (BioNV) comprising:
(i) one or more membrane-embedded targeting agents targeted against one or more markers of cellular infection with the virus;
(ii) one or more membrane-embedded proteins of an α-phagocytic integrin, CCL2, H2-M3, FasL, MFG-E8, PD-L1 and/or CTLA-4, and SerpinB9;
(iii) a membrane-embedded protein of one of either CD24, CD47, CD200, chimeric CD24/CD47, chimeric CD24/CD200, and chimeric CD47/CD200, or two of either CD24, CD47, and CD200; and
(iv) a membrane substantially lacking proteins of one or more of HLA-A, HLA-B, HLA-C, HLA-F, CIITA, IL-6, TRAC, TRBC, one of either HLA-E or HLA-G, and one or more of IL-4, IL-10, and/or IL-16; and; and
a checkpoint inhibiting agent.

108. A composition for the treatment of a viral infection comprising:
an allogeneic, hypoimmunogenic biomimetic nanovesicle (BioNV) comprising:
(i) one or more membrane-embedded targeting agents targeted against one or more markers of cellular infection with the virus;
(ii) one or more membrane-embedded proteins of an α-phagocytic integrin, CCL2, H2-M3, FasL, MFG-E8, PD-L1 and/or CTLA-4;
(iii) a membrane-embedded protein of either chimeric CD24/CD47, or CD24 and CD47; and
(iv) a membrane substantially lacking proteins of one or more of HLA-A, HLA-B, HLA-C, HLA-F, CIITA, IL-6, TRAC, TRBC, SerpinB9, CD200, one of either HLA-E or HLA-G, and one or more of IL-4, IL-10, and/or IL-16; and
a checkpoint inhibiting agent.

109. The composition of claim 107 or 108, wherein the BioNV encapsulates a gene editing payload.

110. The composition of claim 109, wherein gene editing payload comprises one or more gene editor nucleic acids and/or proteins, or one or more nucleic acids encoding one or more gene editors.

111. The method of claim 110, wherein the one or more gene editors is a site-directed endonuclease, TALEN, ZFN, RNase P RNA, CRISPR/Cas nuclease, C2c1, C2c2, C2c3, Cas9, Cpf1, TevCas9, Archaea Cas9, CasY.1, CasY.2, CasY.3, CasY.4, CasY.5, CasY.6, CasX, Cas omega, transposase, and/or any ortholog or homolog thereof.

112. The method of claim 111, wherein the gene editing payload comprises a transactivating response region (TAR) loop system.
